(19) United States
(12) Patent Application Publication
(10) Pub. No.: US 2026/0273221 A1
(43) Pub. Date: Sep. 17, 2026

(54) MATERIAL BINDING COMPOSITIONS AND RELATED METHODS

(71) Applicant: Access Vascular, Inc., Billerica, MA (US)

(72) Inventors: Daniel T. Donahue, Cambridge, MA (US); Nikolaos Pipis, Gainesville, FL (US); Mark Thomas Doherty, Lynnfield, MA (US)

(73) Assignee: Access Vascular, Inc., Billerica, MA (US)

(21) Appl. No.: 19/163,800

(22) PCT Filed: Mar. 8, 2024
(86) PCT No.: PCT/US2024/019063

§ 371 Date: Sep. 9, 2025

Related U.S. Application Data
(60) Provisional application No. 63/451,470, filed on Mar. 10, 2023.

(51) Int. Cl. A61M 25/00

(52) U.S. Cl. CPC A61M 25/0043; A61M 25/007; A61M 2025/006; A61M 2205/0227; A61M 2205/0233

(57) ABSTRACT

Articles and/or integrated articles comprising a body portion (e.g., a catheter) and a component (e.g., a cuff) physically integrated with the body portion arc generally provided. For example, materials, methods, and uses are set forth herein for forming a body portion comprising a first material physically integrated with a component comprising a second material, same or different, than the first material. The disclosed compositions and devices may be useful for administration to a subject (e.g., a patient). Advantageously, the compositions and/or devices described herein may be substantially non-thrombogenic, lubricious, and/or biocompatible. In some embodiments, the devices described herein may be useful for the delivery of a biologically active agent (e.g., a therapeutic agent such as a drug) to a subject. Methods for forming such compositions and/or devices are also provided.

### TECHNICAL FIELD

The technical field generally relates to porous biomaterials, including high strength hydrophilic nanoporous biomaterials, e.g., comprising catheters and/or polymeric materials.

### BACKGROUND

Biomaterials with high strength, low thrombogenicity and lubricious surface properties are useful in the medical arts. The porosity of the biomaterials allows for both high strength bulk materials for medical devices and channels that permit physical bonding to other materials. These properties may prevent or reduce biofilm, microbial colonization, infection, fibrin sheath formation, inflammation, pain, and/or tumor growth, and/or may treat physiological conditions such as tumor reduction, fungal and bacterial infections, inflammation, and pain. Complications seen with such devices lengthen hospital stays and increase patient morbidity and mortality.

Accordingly, improved devices and methods are needed.

### SUMMARY

Articles and/or integrated articles comprising a body portion (e.g., a catheter) are generally provided. In some aspects, material binding compositions and related methods are provided. In some aspects, catheter tip formation methods are provided. In some aspects, intravenous catheters and related methods are provided.

For example, in some embodiments, materials, methods, and uses are set forth herein for forming a body portion comprising a first material physically integrated with a component comprising a second material, same or different, than the first material. The disclosed compositions and devices may be useful for administration to a subject (e.g., a patient). Advantageously, the compositions and/or devices described herein may be substantially non-thrombogenic, lubricious, and/or biocompatible. In some embodiments, the devices described herein may be useful for the delivery of a biologically active agent (e.g., a therapeutic agent such as a drug) to a subject. Methods for forming such compositions and/or devices are also provided.

Some aspects of the disclosure relate to articles comprising a body portion a body portion, wherein the body portion comprises a first material comprising a water-soluble polymer; and a component physically integrated with the body portion. In some embodiments, the component comprises a second material that is different than the first material. In some embodiments, one or more of the following holds: (i) the first material has a Young's elastic modulus of greater than or equal to 500 MPa in a dehydrated state and a Young's elastic modulus of less than or equal to 300 MPa and greater than or equal to 5 MPa at an equilibrium water content state; (ii) the first material is configured to swell in an amount greater than or equal to 5 w/w % and less than or equal to 50 w/w % from a dehydrated state to an equilibrium water content state in less than or equal to 60 minutes at 25° C.; (iii) the first material is free of covalent crosslinks between the water-soluble polymer that forms the first material; and/or (iv) the first material comprises pores that each have a diameter of 1 μm or less.

Some aspects of the disclosure relate to articles comprising a hydrophilic porous polymeric material comprising a water-soluble polymer. In some embodiments, the hydrophilic porous polymeric material comprises a plurality of pores. In some embodiments, the hydrophilic porous polymeric material is free of covalent crosslinks between the water-soluble polymer. In some embodiments, a component is physically integrated with the hydrophilic porous polymeric material. In some embodiments, the component comprises a second material different than the first material. In some embodiments, the component comprises a plurality of fibers. In some embodiments, at least a portion of the plurality of fibers are embedded within the hydrophilic porous polymeric material.

Some aspects of the disclosure relate to articles comprising a body portion, wherein the body portion comprises a first material comprising a water-soluble polymer. In some embodiments, the first material comprises a plurality of pores and a component is physically integrated with the body portion such that at least a portion of the component is embedded within the body portion. In some embodiments, the component comprises a second material different than the first material.

Some aspects of the disclosure relate to methods of forming integrated articles. In some embodiments, the methods comprise with a polymeric mixture comprising at least one water-soluble polymer and a solvent, the polymeric mixture having a concentration of at least 10% w/w of the at least one water-soluble polymer, performing the steps of: (i) heating the polymeric mixture to achieve a temperature above the melting point of the polymeric mixture,
extruding the polymeric mixture as a body portion; and (ii) removing the solvent from the extruded body portion at a temperature above a freezing point of the solvent until the body portion is porous. In some embodiments, the body portion is a porous body portion that comprises the at least one water-soluble polymer, and the porous body portion is made without covalent crosslinking agents that form covalent crosslinks between polymers extruded to make the porous body portion. In some embodiments, during the step of extruding the polymer mixture, a component is physically integrated with the body portion, the component comprising a second material different than the first material, thereby forming the integrated article.

Additional aspects of the disclosure relate to methods of forming an integrated article comprising exposing a body portion to a solvent; and optionally, heating the body portion to a temperature of less than or equal to 100° C. In some embodiments, the body portion comprises a first material comprising a water-soluble polymer, such that, in the presence of the solvent, the water-soluble polymer softens. In some embodiments, the methods comprise contacting the body portion with a component in the presence of the solvent, such that the component physically integrates with the body portion thereby forming the integrated article. In some embodiments, the methods comprise cooling the integrated article.

In some embodiments, the methods comprise exposing a body portion to a solvent and contacting the body portion with a component in the presence of the solvent, such that the component physically integrates with the body portion thereby forming the integrated article. In some embodiments, the body portion comprises a first material comprising a first water-soluble polymer. In some embodiments, the solvent comprises water and a second water-soluble polymer. In some embodiments, the methods comprise as least partially drying the integrated article.

Other aspects of the disclosure relate to articles comprising a body portion, a solution coating disposed on a portion of a surface of the body portion, and a component in physical contact with the solution coating, such that the component is adhered to the body portion via the solution coating. In some embodiments, the body portion comprises a first material comprising a water-soluble polymer. In some embodiments, the solution coating comprises water and polyvinyl alcohol (PVA). In some embodiments, the PVA is present in the solvent in an amount greater than or equal to 0.1 wt % and less than or equal to 25 wt %.

Other aspects of the disclosure relate to methods for reshaping a hydrophilic porous material. In some embodiments, the methods comprise bending the hydrophilic porous material in a desired shape, wherein the hydrophilic porous material comprises a lumen. In some embodiments, the methods comprises heating the hydrophilic porous material in the bent configuration to greater than or equal to 90° C., wherein: (a) the step of bending the hydrophilic porous material comprises pressing the hydrophilic porous material into a mold having the desired shape; and/or (b) the step of bending the hydrophilic porous material comprises inserting a material into the lumen of the hydrophilic porous material thereby providing the desired shape; and/or (c) the step of bending the hydrophilic porous material comprises physically deforming the hydrophilic porous material and wherein, for any of (a)-(c), one or more of the following holds: (i) the hydrophilic porous material has a Young's elastic modulus of greater than or equal to 500 MPa in a dehydrated state and a Young's elastic modulus of less than or equal to 300 MPa and greater than or equal to 5 MPa at an equilibrium water content state; (ii) the hydrophilic porous material is configured to swell in an amount greater than or equal to 5 w/w % and less than or equal to 50 w/w % from a dehydrated state to an equilibrium water content state in less than or equal to 60 minutes at 25° C.; (iii) the hydrophilic porous material is free of covalent crosslinks between the water-soluble polymer that forms the hydrophilic porous material; and (iv) the hydrophilic porous material comprises pores that each have a diameter of 1 μm or less.

Aspects of the disclosure relate to articles comprising an elongated tube, wherein the elongated tube comprises a first material comprising a water-soluble polymer; the elongated tube comprising a first portion and a second portion, wherein the first portion comprises a radius of curvature different than a radius of curvature of the second portion, in a relaxed state of the elongated tube, wherein one or more of the following holds: (i) the first material has a Young's elastic modulus of greater than or equal to 500 MPa in a dehydrated state and a Young's elastic modulus of less than or equal to 300 MPa and greater than or equal to 5 MPa at an equilibrium water content state; (ii) the first material is configured to swell in an amount greater than or equal to 5 w/w % and less than or equal to 50 w/w % from a dehydrated state to an equilibrium water content state in less than or equal to 60 minutes at 25° C.; (iii) the first material is free of covalent crosslinks between the water-soluble polymer that forms the first material; and (iv) the first material comprises pores that each have a diameter of 1 μm or less.

Aspects of the disclosure relate to dual lumen articles. In some embodiments, the dual lumen article comprises a first body portion comprising a first material comprising a water-soluble polymer, the first body portion comprising a first lumen having a first inner diameter; and a second body portion comprising a second material, different than the first material, the second body portion comprising a second lumen, wherein a first portion of the second body has an outer diameter less than the first inner diameter of the first lumen, and a second portion of the second body has a second inner diameter about equal to the first inner diameter, wherein the second body portion is at least partially disposed within the first body portion, wherein one or more of the following holds: (i) the first material has a Young's elastic modulus of greater than or equal to 500 MPa in a dehydrated state and a Young's elastic modulus of less than or equal to 300 MPa and greater than or equal to 5 MPa at an equilibrium water content state; (ii) the first material is configured to swell in an amount greater than or equal to 5 w/w % and less than or equal to 50 w/w % from a dehydrated state to an equilibrium water content state in less than or equal to 60 minutes at 25° C.; (iii) the first material is free of covalent crosslinks between the water-soluble polymer that forms the first material; and (iv) the first material comprises pores that each have a diameter of 1 μm or less.

Some aspects of the disclosure also relate to methods of forming a dual lumen article. In some embodiments, the methods comprise swelling, in a solvent, a first body portion comprising a first material comprising a water-soluble polymer, the first body portion comprising a first lumen having a first inner diameter; heating and/or mechanically deforming a second body portion, the second body portion comprising a second material different than the first material, such that the second body portion has an outer diameter less than the first inner diameter of the first lumen; inserting the second body portion into the lumen of the first body portion thereby forming the dual lumen article; and drying the dual lumen article such that the first body portion shrinks.

Additional aspects of the disclosure relate to intravenous catheters. In some embodiments, the intravenous catheter comprises a polymeric material comprising a first water-soluble polymer having a plurality of pores; a lumen; and a distal end comprising a tip geometry suitable for intravenous insertion into a subject; wherein the polymeric material has a water content of less than 5 w/w % and greater than or equal to 0.1 w/w % in a dehydrated state, and wherein the polymeric material is configured to swell in an amount greater than or equal to 5 w/w % and less than or equal to 50 w/w % from a dehydrated state to an equilibrium water content state in less than or equal to 60 minutes at 25° C.

Other aspects of the disclosure relate to systems comprising an intravenous catheter and a component configured for administering a therapeutic agent and/or fluid to a subject, and/or drawing a bodily fluid from the subject.

Aspects of the disclosure further relate to methods of inserting an intravenous catheter into a subject in need thereof. In some embodiments, the methods comprise inserting, intravenously, an intravenous catheter, the intravenous catheter comprising: a polymeric material comprising a first water-soluble polymer having a plurality of pores; a lumen; and a distal end comprising a tip geometry suitable for intravenous insertion into a subject; wherein, during the step of insertion, the catheter has a water content of less than or equal to 5 w/w %; and swelling the intravenous catheter to a water content greater than or equal to 5 w/w % and less than or equal to 50 w/w %.

In some embodiments, the methods comprise inserting, intravenously, an intravenous catheter, the intravenous catheter comprising: a polymeric material comprising a first water-soluble polymer having a plurality of pores, wherein the polymeric material has a water content greater than or equal to 5 w/w % and less than or equal to 50 w/w %; a lumen; and a distal end comprising a tip geometry suitable for intravenous insertion into a subject; wherein one or more of the following holds: (i) the polymeric material has a Young's elastic modulus of greater than or equal to 500 MPa in a dehydrated state and a Young's elastic modulus of less than or equal to 300 MPa and greater than or equal to 5 MPa at an equilibrium water content state; (ii) the polymeric material is free of covalent crosslinks between the water-soluble polymer that forms the polymeric material; and (iii) the polymeric material comprises pores that each have a diameter of 1 μm or less.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic diagram of an exemplary article comprising a body portion comprising a plurality of pores and a component, physically integrated with the body portion, according to one set of embodiments;

FIG. 2 is a schematic diagram of an exemplary article comprising a body portion comprising a plurality of pores and a component comprising a plurality of fibers, wherein the fibers of the component are entangled within the pores of the body portion, according to one set of embodiments;

FIG. 3 is a schematic diagram of two materials that are physically integrated with each other, according to one set of embodiments;

FIG. 4 is a schematic diagram of two materials that are physically integrated with each other using a solution coating, according to one set of embodiments;

FIG. 5A is a schematic diagram of a shaped catheter comprising an elongated tube, according to one set of embodiments;

FIG. 5B is a schematic diagram of a cross-section of a shaped catheter shown in FIG. 5A, according to one set of embodiments;

FIG. 6A is a schematic diagram of a horizontal cross-section of an exemplary dual lumen article, according to one set of embodiments;

FIG. 6B is a schematic diagram of a vertical cross-section of the dual lumen article shown in FIG. 6A, according to one set of embodiments;

FIG. 7 is a schematic diagram of an exemplary intravenous catheter comprising a distal end with a tip geometry suitable for intravenous insertion, according to one set of embodiments;

FIG. 8A is a schematic diagram of an exemplary catheter comprising an inflatable cuff, according to one set of embodiments;

FIG. 8B is a schematic diagram of the cross-section of the catheter shown in FIG. 8A in the collapsed view and the inflated view, according to one set of embodiments;

FIG. 9A is a schematic of an extrusion apparatus to form a continuous form with a cut-away view of a side of the bath, according to one set of embodiments;

FIG. 9B is an enlarged view of a portion of the apparatus of FIG. 9A depicting the die head in perspective as viewed from the outside of the bath, according to one set of embodiments;

FIG. 9C is an enlarged view of a portion of the apparatus of FIG. 9A depicting the die head as disposed in the bath, according to one set of embodiments;

FIG. 10 is a longitudinal cross section of a portion of a continuous porous solid as formed with the apparatus of FIGS. 9A-9C, according to one set of embodiments;

FIG. 11A is a schematic of a process of bulk incorporation of a polymer into a porous solid; according to one set of embodiments;

FIG. 11B is a cross-section of a portion of a tube taken along line 3B-3B of FIG. 11A, according to one set of embodiments;

FIG. 12 is a process flow chart for an embodiment of bulk incorporating a surface polymer into a porous solid and includes an extrusion process for making the porous solid, according to one set of embodiments; and

FIG. 13 is a plot depicting a stress-strain curve of a polymeric material, according to one set of embodiments.

FIGS. 14A-14C are photographs of exemplary shaped catheters, according to some embodiments. FIG. 14A is a photograph of a partially hydrated straight catheter. FIG. 14B is a photograph of a partially hydrated straight catheter (in 2.2% sodium chloride) on shaped mandrel. FIG. 14C (above) shows the shaped mandrel. FIG. 14C (below) is a photograph of an exemplary catheter dried 95° C., 90m minutes and annealed at 150° C., 90 minutes, rehydrated in 1× phosphate buffered saline 37° C., with the mandrel removed.

FIGS. 15A-15C are photographs of an exemplary reshaping of a lumen from a circle to a D-shape, according to one set of embodiments. FIG. 15A shows room temp/20° C. (no change); FIG. 15B shows 70° C., 65% of mandrel aspect ratio; and FIG. 15C shows 95° C., 87% of mandrel aspect ratio.

### DETAILED DESCRIPTION

Articles and/or integrated articles comprising a body portion (e.g., a catheter) and a component (e.g., a cuff) physically integrated with the body portion are generally provided. For example, materials, methods, and uses are set forth herein for forming a body portion comprising a first material physically integrated with a component comprising a second material, same or different, than the first material. The disclosed compositions and devices may be useful for administration to a subject (e.g., a patient). Advantageously, the compositions and/or devices described herein may be substantially non-thrombogenic, lubricious, and/or biocompatible. In some embodiments, the devices described herein may be useful for the delivery of a biologically active agent (e.g., a therapeutic agent such as a drug) to a subject. Methods for forming such compositions and/or devices are also provided.

The articles described herein may be useful for a wide variety of applications including, for example, to make blood-contacting devices or devices that contact bodily fluids, including ex vivo and/or in vivo devices, such as blood contacting implants. Examples of drug delivery devices in which the articles described herein may embody or be incorporated into include but are not limited to medical tubing, wound dressing, contraceptive devices, feminine hygiene, endoscopes, grafts (e.g., including small diameter of less than or equal to 6 mm), pacemakers, implantable cardioverter-defibrillators, cardiac resynchronization devices, cardiovascular device leads, ventricular assist devices, catheters (e.g., including cochlear implants, endotracheal tubes, tracheostomy tubes, ports, shunts), implantable sensors (e.g., intravascular, transdermal, intracranial), ventilator pumps, and ophthalmic devices including drug delivery systems.

In one set of embodiments, physically integrated articles are provided. In some embodiments, the article comprises a body portion and a component physically integrated with the body portion. Methods for forming physically integrated articles are also provided. For example, in some embodiments the integrated articles are formed using extrusion, thermal bonding, and/or solvent bonding.

In another set of embodiments, shaped articles are provided. In some embodiments the shaped article comprises an elongated tube, wherein at least a portion of the elongated tube has a particular radius of curvature. Methods of forming shaped articles are also provided. For example, in some embodiments the article may be shaped using thermal forming, for example, by placing a wire (nitinol, steel, etc.) inside a lumen and thermally heating the article to mold it into the desired shape. The shaped article may have any suitable shape including, but not limited to, straight, curved, bent, zig-zag, spiral, looped, corkscrewed, waved, irregular, and twisted, amongst others.

In yet another set of embodiments, dual lumen articles are provided. In some embodiments, the dual lumen article comprises a first body portion and a second body portion, the second body portion at least partially disposed within the first body portion. Methods for forming dual lumen articles are also provided. For example, in some embodiments the dual lumen article may be formed by swelling a first body portion in a solvent until the first body portion is deformable and inserting a second body portion into the lumen of the first body portion. In some cases, the second body portion may also be deformed using heat and or mechanical force.

In a further set of embodiments, intravenous catheters are provided. In some embodiments, the intravenous catheters comprise a body portion (e.g., a polymeric material) comprising a first water-soluble polymer. In some embodiments the polymeric material has a plurality of pores. In certain embodiments, the intravenous catheter comprises a lumen and a distal end comprising a tip geometry suitable for intravenous insertion into a subject (e.g., a bevel tip needle stylet). Methods of inserting an intravenous catheter into a subject in need are also provided. For example, in some embodiments, the intravenous catheter is inserted (e.g., percutaneously) in the dehydrated state and becomes hydrated after insertion; in certain embodiments, the catheter is inserted in the hydrated state. In some embodiments, the hydrated intravenous catheters are softer and do not cause discomfort associated with traditional polymeric catheters.

Some of the embodiments described above utilize a solvent. For example, in some embodiments a solvent is used to solubilize at least a portion of polymers within a first and/or second material. In certain embodiments, the solvent is used to swell a material rendering it deformable (e.g., bending a catheter to a particular shape or stretching the inner diameter of a lumen). In another set of embodiments, a solvent is used to indirectly bond a first material to a second material (e.g., first material is bonded to the solvent and the solvent is bonded to the second material).

In some embodiments, the solvent comprises a water-soluble polymer (e.g., polyvinyl alcohol) dissolved in a aqueous solution. In some embodiments, the solvent comprises an aqueous solution (e.g., without a water-soluble polymer). In certain embodiments, the solvent comprises water-miscible organic compounds (e.g., DMSO).

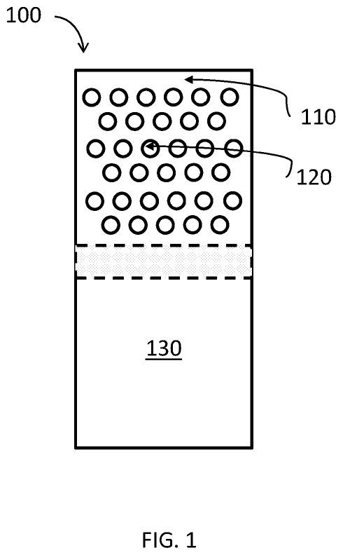

FIG. 1 illustrates an exemplary embodiment of an integrated article comprising a body portion and a component. For example, integrated article 100 comprises a body portion 110 and a component 130 physically integrated with the body portion. In some embodiments, body portion 120 is formed of a first material and comprises a polymeric material. The polymeric material may comprise a first water-soluble polymer. In some embodiments, the component 130 is formed of a second material different than the first material. In some embodiments, the body portion comprises a plurality of pores 120 (e.g., a plurality of pores having mean pore size of 1 micron or less).

While body portion 110 in FIG. 1 is depicted as rectangular, those of ordinary skill in the art would understand, based upon the teachings of this specification, that the body portion and other sections in embodiments disclosed herein need not be rectangular, and other cross-sectional shapes (e.g., planar, circular, square, oval, oblong, S-shaped, etc.) are also possible. For example, in some embodiments, the body portion is S-shaped, which can, in some cases, provide ease of implantation in a subject, achieve lower infiltration rates, and reduce the likelihood of dislodgement within the subject.

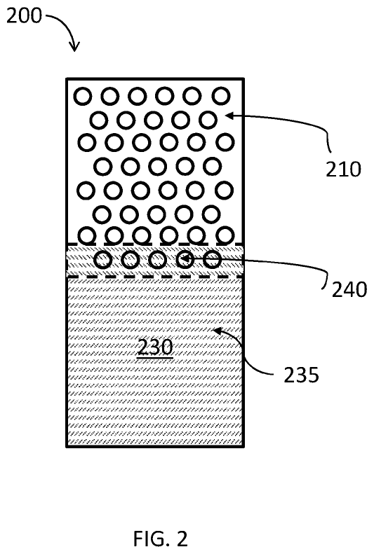

FIG. 2 shows an integrated article 200 comprising a body portion 210 (e.g., polymeric material) comprising one or more components 230 that are physically integrated with the body portion 210 (e.g., a catheter cuff). In some cases, the body portion 210 comprises a first material comprising a hydrophilic water-soluble polymer. In some embodiments, the component 230 comprises a second material comprising a plurality of fibers 235 that are at least partially embedded within the body portion (e.g., first hydrophilic porous material). While component 230 in FIG. 2 is depicted as fibers 235, those of ordinary skill in the art would understand, based upon the teachings of this specification, that the component and other sections in embodiments disclosed herein need not be fibers, and other textiles (e.g., meshes, cellular foam cavities, nettings, etc.) are also possible.

As described herein, in some aspects, at least a portion of the article may be physically integrated. The term ‘physically integrated’ is given its typical meaning in the art and generally refers to the physical joining of two or more components (e.g., layers, articles, surfaces) such that they are adhered together. In some embodiments, two or more components that are physically integrated with one another can generally not be separated without irreversibly damaging at least a portion of one or more of the two or more components (e.g., by breaking, mechanical failure, and/or dissolution of chemical bonds). For example, two or more components that are physically integrated share at least one surface at which the two or more components are adhered to one another (e.g., fused together). Those skilled in the art would understand that the phrases “physically integrated” and “fused together” do not refer to components that simply contact one another at one or more surfaces, but components wherein at least a portion of the original surface of each individual components can no longer be discerned from the other component (e.g., an intermediate layer is formed comprising at least a portion of the first component and at least a portion the second component). In some embodiments, two or more components are physically integrated through bonding, entanglement, pore filling, or the like.

In some embodiments, a first component is physically integrated with a second component (e.g., a body portion) by embedding at least a portion of the first component in the second component. In some embodiments, a first component is physically integrated with a second component (e.g., a body portion) by embedding at least a portion of the second component in the first component. In some embodiments, the first component is physically integrated with the second component via formation of a bond, such as an ionic bond, a covalent bond, a hydrogen bond, Van der Waals interactions, and the like. The covalent bond may be, for example, carbon-carbon, carbon-oxygen, oxygen-silicon, sulfur-sulfur, phosphorus-nitrogen, carbon-nitrogen, metal-oxygen, or other covalent bonds. The hydrogen bond may be, for example, between hydroxyl, amine, carboxyl, thiol, and/or similar functional groups. In some embodiments, the first component is physically integrated with the second component via chain entanglement (e.g., between polymer chains of the first component and polymer chains of the second component).

In an exemplary set of embodiments, physically integrated comprises hydrophilic polymer chains of a first material becoming physically entangled within pores of a second material. In another set of exemplary embodiments, the hydrophilic polymers of the first material may become entangled within the polymer chains of a second material. In yet another set of exemplary embodiments, physically integrated comprises the entanglement of a second material (e.g., plurality of fibers, meshes, cellular foam cavities, nettings, or other structures) within the pores and/or bulk of the first material (e.g., a hydrogel).

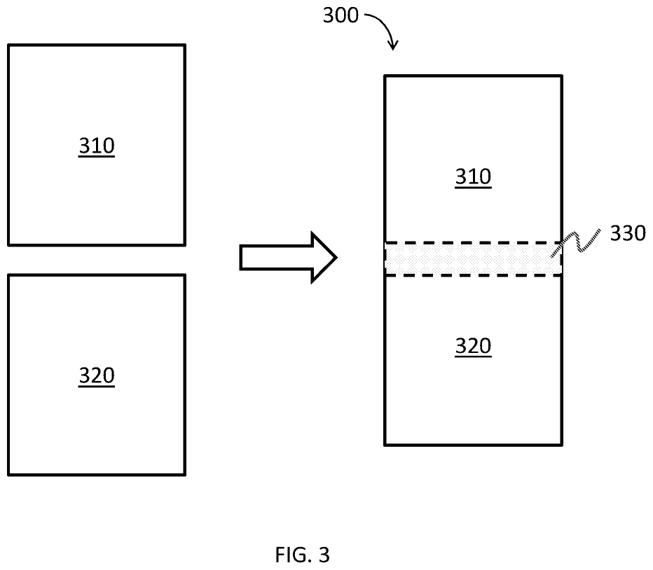

FIG. 3 shows an example of two components, component 310 and component 320, which are physically integrated with each other (e.g., in the presence of one or more solvents as disclosed herein) to form integrated article 300. Integrated article 300, in some embodiments, comprises at least a portion of component 310, at least a portion of component 320, and a physically integrated region 330 (e.g., which comprises a material of component 310 and a material of component 320).

In some embodiments, a solution coating is used to physically integrate a body portion with a component. For example, as shown illustratively in FIG. 4, solution coating 440 is added to the first surface of body portion 410 and placed into physical contact with component 420 to yield integrated article 400. In some embodiments, the solution coating comprises a water-soluble polymer configured to entangle within the pores and/or polymer chains of the first material of the body portion. The solution coating, may also, according to certain embodiments, become entangled or embedded within the pores and/or polymer chains of the second material of the component. In this way, the component is adhered to the body portion via the solution coating (e.g., the body portion is physically integrated with the solution coating which is physically integrated with the component).

In some embodiments, the first component is adjacent the second component. In some embodiments, an intervening layer may be present between the first component and the second component, the intervening layer comprising the solution coating.

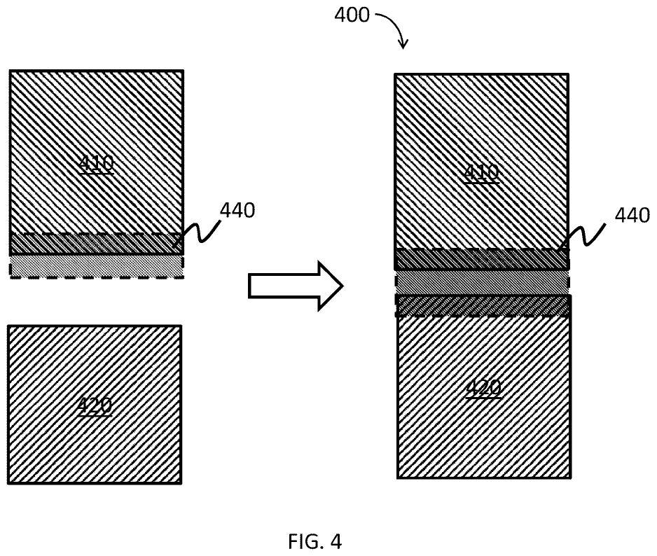

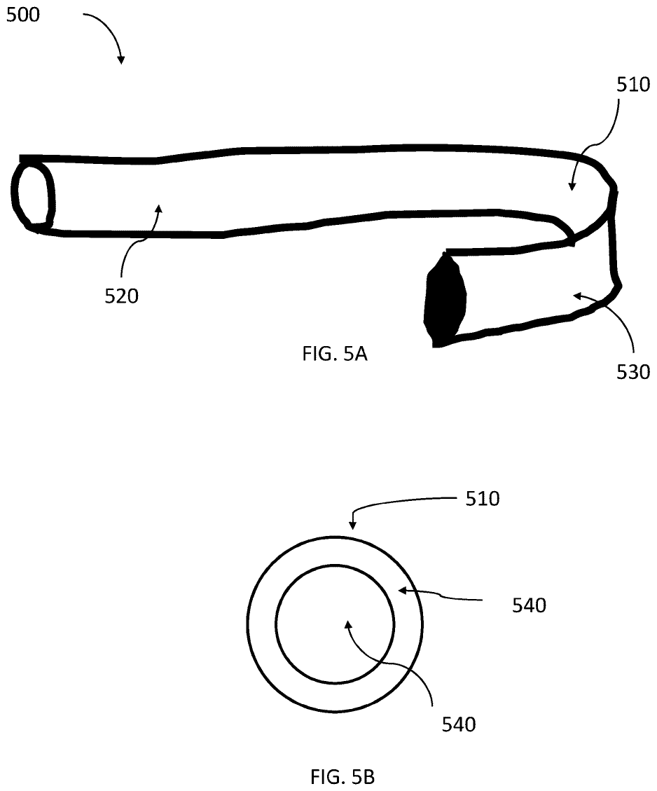

Other aspects of the disclosure relate to articles such as a shaped catheter. For example, as shown illustratively in FIG. 5A, a shaped catheter may comprise an elongated tube 510 comprising a first portion 520 and a second portion 530. In some embodiments, the first portion 520 comprises a radius of curvature different than the radius of curvature of the second portion 530 (e.g., the catheter has a “hooked” shape). FIG. 5B shows a cross-section of exemplary catheter 500. In some embodiments, the elongated tube 510 comprises a lumen 540 and an outer shell comprising a first material 550 comprising a water-soluble polymer. In some embodiments, the first material comprises a plurality of pores with a mean diameter of 1 micron or less.

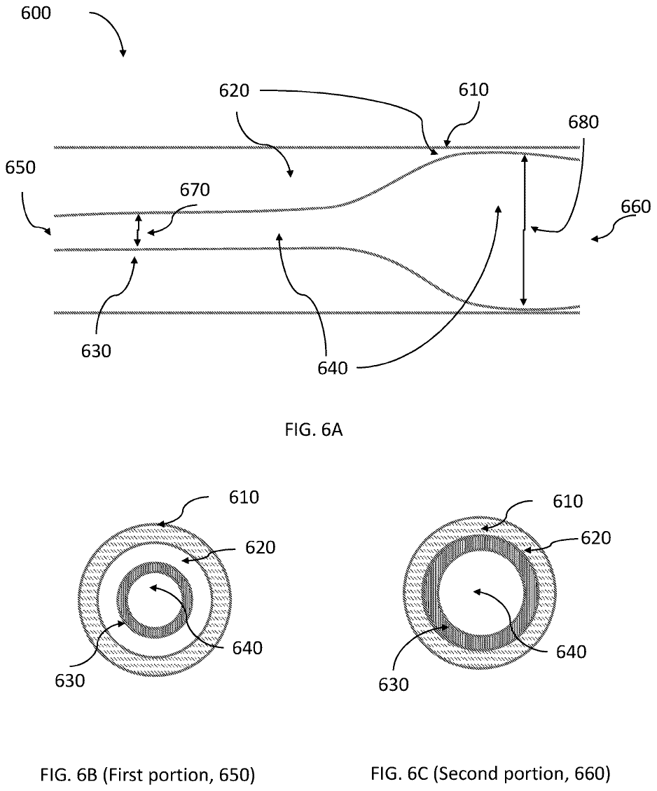

In some embodiments, the article is a dual lumen article. An exemplary embodiment is shown in FIG. 6. FIG. 6A shows a longitudinal cross-section of a dual lumen article comprising a first body 610 comprising a first lumen 620 having a first inner diameter and a second body 630 comprising a second material comprising a second lumen 640. In some embodiments, the second body 630 comprises a first portion 650 that has an outer diameter less than the first inner diameter 670 of the first lumen 620; in some embodiments, a second portion 660 of the second body 630 has a second inner diameter 680 that is about equal to the first inner diameter of the first lumen 620.

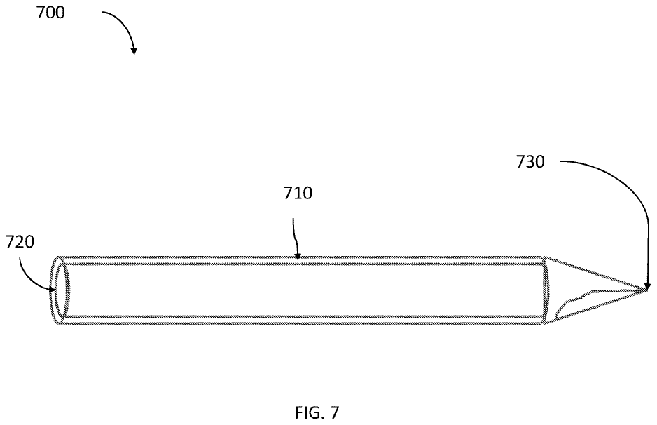

Additionally, in certain embodiments, the article may comprise an intravascular catheter. For example, as shown illustratively in FIG. 7, an intravenous catheter may comprise a polymeric material 710 comprising a first water-soluble polymer having a plurality of pores (e.g., a first body), a lumen 720, and a distal end 730 comprising a tip geometry suitable for intravenous insertion into a subject.

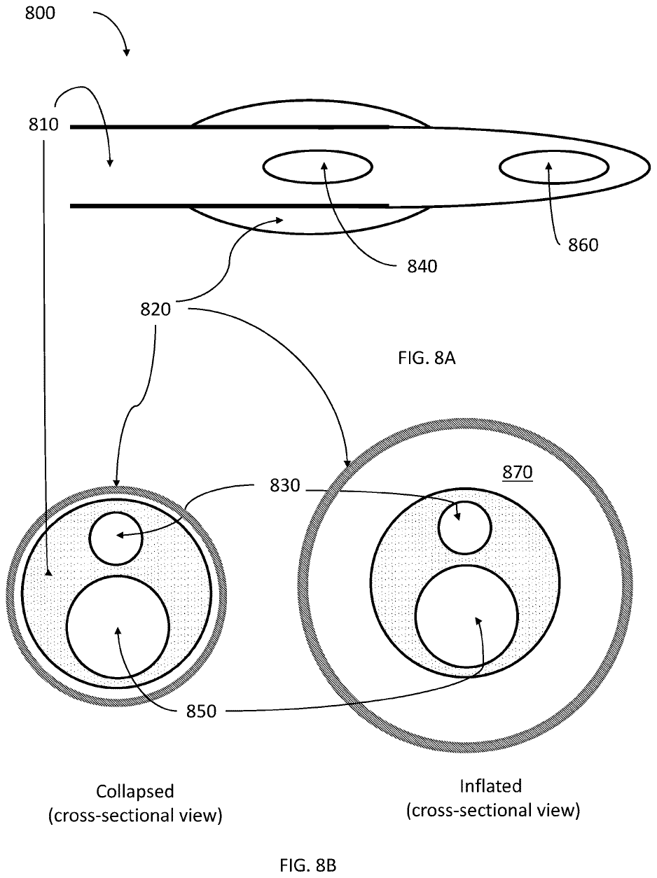

In an exemplary set of embodiments, the article comprises a catheter comprising a cuff, for example, to secure the catheter within a subject's body cavity (e.g., urinary cavity). For example, as shown illustratively in FIG. 8, a catheter 800 may comprise a first material 810 comprising a first water-soluble polymer having a plurality of pores, a second material 820 (e.g., balloon or cuff), a first lumen 830 connected to a first opening 840, a second lumen 850 connected to a second opening 860, and a distal end comprising a tip geometry suitable for the desired application (e.g., blunt tip for urinary applications versus pointed tip for intravascular applications). In some embodiments, the second material is physically integrated with the first material along its perimeter such that the center of the second material is positioned over the first opening. In some embodiments, the second material is configured to expand radially upon introduction of a gas 870 (e.g., air) into the first lumen (e.g., via a syringe).

Some of the embodiments of the instant application utilize a solvent (e.g., to bond a first material to a second material). In some embodiments, the solvent comprises water. In some embodiments, the solvent comprises a water-soluble polymer (e.g., a second water-soluble polymer). In some embodiments, the solvent comprises a water-soluble organic compound (e.g., DMSO).

Without wishing to be bound by any particular theory, it is believed that contacting the body portion with a component in the presence of a solvent (e.g., water) results in the interdiffusion of the first water-soluble polymer of the body portion into the second material of the component, thus forming an integrated article. In some embodiments, the method results in interdiffusion of the second material into the first material of the body portion (e.g., when the second material is the same as the first material). Combinations are also possible. For example, in some embodiments, the first material may diffuse into the second material, and the second material may also diffuse into the first material.

In some embodiments, a solvent comprising water and a second water-soluble polymer may be used to physically integrate the component comprising a second material with the body portion comprising a first material. Again, without wishing to be bound by any particular theory, it is believed that contacting the body portion with a component in the presence of the solvent results in the interdiffusion of the second water-soluble polymer into the first material of the body portion and the second material of the component, thus forming an integrated article. In other words, the solvent may act as a polymer bridge that becomes physically integrated with both the body portion and the component, thus physically integrating the body portion and the component. For example, in some embodiments, the second water-soluble polymer (e.g., poly(vinyl alcohol) may be entrained within the body portion and one or more components (e.g., Dacron® (Poly ethylene terephthalate aka polyester), electrospun PVA, cotton, wool, Polypropylene (atactic, syndiotactic, isotactic), and polyethylene (LLDPE, LDPE, HDPE)).

As described above and herein, the solvent may interact, in some embodiments, with more than one element of a body portion or component. For example, in some cases, the solvent may physically integrate the body portion with a component by becoming entangled within the surface, bulk, and/or pores of the body portion and the surface, bulk, and/or pores of the component.

In some embodiments, the solvent comprises a homogenous solvent (e.g., water, DMSO, etc.,) or a heterogenous mixture (e.g. a co-solvent). Exemplary embodiments of solvents include, but are not limited to, water and DMSO. For example, in some embodiments, the solvent is a heterogeneous mixture of water/DMSO having a ratio of about 0:1, 0.1:0.9, 0.2:0.8, 0.3:0.7, 0.4:0.6, 0.5:0.5, 0.6:0.4, 0.7:0.3, 0.8:0.2, 0.9:0.1, 1:0 (or combinations thereof or ranges thereof).

In some embodiments, the solvent comprises a polymer. The polymer may be the same, or different, as the first material (e.g., a second water-soluble polymer). For example, in some embodiments, the solvent comprises a water-soluble polymer (e.g., a second water-soluble polymer) that comprises or is selected from the group consisting of poly(vinyl alcohol), poly(acrylic acid), polyethylene glycol, poly(vinyl pyrrolidone), poly(methacrylic sulfobetaine), poly(acrylic sulfobetaine), poly(methacrylic carboxybetaine), poly(acrylic carboxybetaine), povidone polyacrylamide, poly(N-(2-hydroxypropyl)methacrylamide), polyoxazolines, polyphosphates, polyphosphazenes, polyvinyl acetate, polypropylene glycol, poly(N-isopropylacrylamide), poly(2-hydroxymethylmethacrylate), and combinations thereof. In a preferred set of embodiments, the solvent comprises poly(vinyl alcohol).

In some embodiments, the water-soluble polymer in the solvent is present at a concentration less than the concentration of the water-soluble polymer used to form the first material. For example, in some embodiments, the water-soluble polymer is present in the solvent in an amount of greater than or equal to 20 w/w %, greater than or equal to 25 w/w %, greater than or equal to 30 w/w %, greater than or equal to 35 w/w %, greater than or equal to 40 w/w %, greater than or equal to 45 w/w %, greater than or equal to 50 w/w %, greater than or equal to 55 w/w %, greater than or equal to 60 w/w %, greater than or equal to 65 w/w %, greater than or equal to 70 w/w %, greater than or equal to 75 w/w %, greater than or equal to 80 w/w %, greater than or equal to 85 w/w %, or greater than or equal to 90 w/w % at an equilibrium water content state. In some embodiments, the water-soluble polymer is present in the solvent in an amount of less than or equal to 95 w/w %, less than or equal to 90 w/w %, less than or equal to 85 w/w %, less than or equal to 80 w/w %, less than or equal to 75 w/w %, less than or equal to 70 w/w %, less than or equal to 65 w/w %, less than or equal to 60 w/w %, less than or equal to 55 w/w %, less than or equal to 50 w/w %, less than or equal to 45 w/w %, less than or equal to 40 w/w %, less than or equal to 35 w/w %, less than or equal to 30 w/w %, or less than or equal to 25 w/w % at an equilibrium water content state. Combinations of the above-referenced ranges are also possible (e.g., greater than or equal to 20 w/w % and less than or equal to 95 w/w %). Other ranges are also possible.

In some embodiments, the solvent comprises water and poly(vinyl alcohol) present in the solvent in an amount of greater than or equal to 0.1 wt % and less than or equal to 25 wt %. For example, in some embodiments. The solvent comprises greater than or equal to 0.1 wt %, greater than or equal to 0.5 wt %, greater than or equal to 1.0 wt %, greater than or equal to 2.0 wt %, greater than or equal to 4.0 wt %, greater than or equal to 8.0 wt %, greater than or equal to 10 wt %, greater than or equal to 12 wt %, greater than or equal to 14 wt %, greater than or equal to 16 wt %, greater than or equal to 18 wt %, greater than or equal to 20 wt %, or greater than or equal to 25 wt % poly(vinyl alcohol) at an equilibrium water content state. In some embodiments, the solvent comprises less than or equal to 25 wt %, less than or equal to 20 wt %, less than or equal to 18 wt %, less than or equal to 16 wt %, less than or equal to 14 wt %, less than or equal to 12 wt %, less than or equal to 10 wt %, less than or equal to 8.0 wt %, less than or equal to 4.0 wt %, less than or equal to 2.0 wt %, less than or equal to 1.0 wt %, less than or equal to 0.5 wt %, or less than or equal to 0.1 wt %, poly(vinyl alcohol) at an equilibrium water content state. Combinations of the above-referenced ranges are also possible (e.g., greater than or equal to 0.1 w/w % and less than or equal to 25 w/w %). Other ranges are also possible.

In some embodiments, the solvent comprises a polymer comprising poly(vinyl alcohol). Poly(vinyl alcohol) may be purchased via commercial vendors (e.g., MilliPore Sigma) and/or synthesized by those skilled in the art, e.g., via hydrolysis of polyvinyl acetate (or other vinyl ester-derived polymers with formate or chloroacetate groups instead of acetate). In some embodiments, the hydrolysis reaction is less than 100%, resulting in a polymer with a mixture of hydroxyl and acetate groups. In certain embodiments, the degree of hydrolysis is greater than to equal to 20% and less than or equal to 99%. For example, in some embodiments, the degree of hydrolysis is greater than or equal to 20%, greater than or equal to 30%, greater than or equal to 40%, greater than or equal to 50%, greater than or equal to 60%, greater than or equal to 70%, greater than or equal to 80%, greater than or equal to 90%, greater than or equal to 95%, or greater than or equal to 99%. In some embodiments, the degree of hydrolysis is less than or equal to 99%, less than or equal to 95%, less than or equal to 90%, less than or equal to 80%, less than or equal to 70%, less than or equal to 60%, less than or equal to 50%, less than or equal to 40%, less than or equal to 30%, or less than or equal to 20%. In certain embodiments, the solvent comprises a poly(vinyl alcohol) polymer that is 80% hydrolyzed, corresponding to about 20% acetate groups and about 80% hydroxyl groups.

The solvent may be applied to a material (e.g., a body portion) using any technique known to those of skill in the art. Exemplary embodiments, include but are not limited to, (1) the brush on method where the solvent is brushed onto the surfaces to be joined with subsequent pressure being applied until full strength of the bond is formed after the solvent have fully evaporated; (2) capillary action method which uses capillary action to wick the solvent into gaps formed at the junction between the two parts to be joined; (3) dip-dab method which dips the surface to be joined into a vat of solvent for a set time prior to pairing with the desired component, and (4) solvent dispenser method in which a dispenser is used to precisely control the amount of solvent applied on each surface to be joined.

Other applications for the solvent are also contemplated. For example, in some embodiments, the solvent may be used to swell and bend the article to any desired shape. Additionally, or alternatively, the solvent may be used to entrain or entrap non-soluble compounds like therapeutics, biologically active compounds, woven textiles, metallic mesh, color indicators, radiopacifiers, reactive indicators and the like, onto the outer surface of the integrated article. In some cases, the solvent may be used to incorporate conductive indicators to aid in Electrocardiogram p-wave detection. For example, a ferrous powder that does not dissolve in physiological fluid entrained in the second water-soluble polymer (e.g., poly(vinyl alcohol) may provide a built in ECG probe (e.g., when placed in the tip of a catheter).

In certain embodiments, the solvent may be used to bond one or more materials to a first water-soluble polymer (e.g., poly(vinyl alcohol). For example, in some cases, the solvent may be used to bond bandages, tissues, stents, shunts, braided tubing, and/or wall stents to one or more materials also comprising a water-soluble polymer (e.g., poly(vinyl alcohol)).

In some embodiments, the solvent may be used in combination with other techniques, such as heating. For example, as described herein and above, bonding may be achieved by applying a solvent to the first and second materials to be bonded together (e.g., in an overlapped joint) and heating the first material and/or second material to allow the polymer chains of the first material to flow into the bulk of the second material, and vise versa. Upon cooling, the first material becomes physically integrated with the second material to form a single cohesive material.

Some aspects of the current disclosure generally relate to methods of producing the integrated articles described herein. In some embodiments, the method comprises heating a polymeric mixture comprising a water-soluble polymer and a solvent and extruding the mixture as a body portion while simultaneously placing the body portion in physical contact with a component, thus physically integrating the body portion and the component.

In some embodiments, the method comprises exposing a body portion to a solvent, and optionally, heating the body portion to a temperature of less than or equal to 100° C., contacting the body portion with a component in the presence of the solvent to form an integrated article, and cooling the integrated article.

In some embodiments, the method comprises exposing a body portion to a solvent to soften a first material of the body portion and contacting the body portion with a component in the presence of the solvent, such that the component physically integrates with the body portion, thereby forming an integrated article, and at least partially drying the article.

Other methods are directed toward shaping articles. For instance, in some embodiments, the method comprises bending a hydrophilic porous material (e.g., body portion) comprising a lumen into a desired shape and heating the material in the bent configuration to greater than or equal to 90° C.

In some embodiments, the method is directed toward formation of a dual lumen article. In certain embodiments, the method comprises swelling a first body portion comprising a first lumen of a first inner diameter in a solvent to soften the first body portion. In some embodiments, the method further comprises heating and/or mechanically deforming a second body portion such that the second body portion has an outer diameter less than the first inner diameter of the first lumen. The method further comprises inserting the second body portion into the lumen of the first body portion thereby forming a dual lumen article.

Other methods still, are directed toward inserting an article (e.g., catheter) into a subject in need thereof. In some embodiments, the article is an intravenous catheter comprising a porous polymeric material comprising a first water-soluble polymer, a lumen, and a distal end comprising a tip geometry suitable for intravenous insertion into a subject. In some embodiments, the article is inserted in the dehydrated state (e.g., the water content of the catheter is less than or equal to 5% wt/wt and becomes hydrated after placement into the subject (e.g., the catheter swells to a water content of greater than or equal to 5% wt/wt and less than or equal to 50% wt/wt).

In some embodiments, the intravenous catheter is hydrated prior to insertion into a subject (e.g., e.g., the catheter swells to a water content of greater than or equal to 5% wt/wt and less than or equal to 50% wt/wt).

Other embodiments have also been contemplated herein. Systems comprising the catheters and a component configured to deliver a therapeutic and/or withdraw a bodily fluid from a subject are also disclosed.

Some aspects of the current disclosure generally relate to compositions, articles, and devices comprising a body portion and a component physically integrated with the body portion. The body portion may be formed of a first material comprising, for example, a first water-soluble polymer (e.g., polyvinyl alcohol). In some embodiments, the first material comprises polymer chains that are non-covalently crosslinked. In certain cases, the first material comprises pores. In some cases, the first material comprises a combination of non-covalently crosslinked polymer chain and pores.

The body portion, may in some cases, further comprises a component comprising a second material that may be physically integrated with the body portion. In some embodiments, the second material may be the same, or different, as the first material. The second material, in some embodiments, comprises a polymer (e.g., a second polymer). In some cases, the polymer comprises a second water-soluble polymer such as polyvinyl alcohol (e.g., PVA). In some embodiments, the polymer comprises a partially-water-soluble polymer; and in certain cases, the polymer may comprise a water-insoluble polymer.

In some embodiments, an article comprises a body portion comprising a first material comprising a water-soluble polymer (e.g., PVA). As described elsewhere herein, the body portion may comprise any water-soluble polymer known to the skilled artisan. Exemplary embodiments include, but are not limited to, poly(vinyl alcohol), poly(acrylic acid), polyethylene glycol, poly(vinyl pyrrolidone), poly(methacrylic sulfobetaine), poly(acrylic sulfobetaine), poly(methacrylic carboxybetaine), poly(acrylic carboxybetaine), povidone, polyacrylamide, poly(N-(2-hydroxypropyl)methacrylamide), polyoxazolines, polyphosphates, polyphosphazenes, polyvinyl acetate, polypropylene glycol, poly(N-isopropylacrylamide), poly(2-hydroxymethylmethacrylate), and combinations thereof. In an exemplary set of embodiments, the first material is poly(vinyl alcohol).

The first material of the body portion, in some embodiments, comprises a Young's elastic modulus of greater than or equal to 500 MPa in a dehydrated state and a Young's elastic modulus of less than or equal to 300 MPa and greater than or equal to 5 MPa at an equilibrium water content state. In some cases, the first material is configured to swell in an amount greater than or equal to 5 w/w % and less than or equal to 50 w/w % from a dehydrated state to an equilibrium water content state in less than or equal to 60 minutes at 25° C. In certain embodiments, first material is free of covalent crosslinks between the water-soluble polymer that forms the first material and/or comprises pores that each have a diameter of 1 μm or less. Other diameters, Young's elastic moduli, and swelling percentages are also contemplated elsewhere herein (see Body Portion section).

The body portion, according to some embodiments, may comprise a hydrophilic porous polymeric material comprising a water-soluble polymer. The hydrophilic porous polymeric material, in some cases, may comprise a plurality of pores. In some embodiments, the hydrophilic porous polymeric material is free of covalent crosslinks between the water-soluble polymer chains and/or comprises a plurality of pores (e.g., pores that have a diameter of 1 μm or less).

The article, according to additional embodiments, may further comprise a component. In some embodiments, the component comprises a second material different, or same, than the first material. In an exemplary set of embodiments, the second material is different than the first material. In other exemplary set of embodiments, the second material is the same as the first material. In some embodiments, the body portion comprises a second material (e.g., component), comprising the first water-soluble polymer at a concentration less than the concentration of the first water-soluble polymer in the first material.

In certain embodiments, the component comprises a second material different, or the same, as the first material. In some cases, the second material comprises a medical grade polymer, metal, or ceramic. The second material may be processed using any technique known to one skilled in the art into a plurality of structures including, but not limited to, a plurality of pores, plurality of fibers, cellular foam cavities, meshes, nettings, and/or other structures (e.g., bulk materials). For instance, in some embodiments, the component may be processed into yarns and fabrics, using techniques known to those of skill in the art, to create complex three-dimensional shapes (e.g., tubular geometries with tapered angles, etc.). In some embodiments, the second material may be a porous structure; in certain embodiments the second material may be a woven structure, in which two sets of polymer yarns are interlaced at right angles. In other embodiments, the second material may be a knit structure, in which loops of polymer yarn are intermeshed; and in some cases, the second material may be braided, in which three or more polymer yarns cross one another in a diagonal pattern, according to other embodiments.

In some embodiments, the article comprises a component physically integrated with a body portion. In certain embodiments, the article comprises a component physically integrated with a hydrophilic porous polymeric material. In some cases, the component is physically integrated with the body portion such that at least a portion of the component is embedded within the body portion.

In certain embodiments, the article comprises a solution coating disposed on a portion of the surface of the body portion configured to configured to physically integrate the body portion and the component to form an integrated article. In some embodiments, the solution coating comprises water and poly(vinyl alcohol) (e.g., PVA) at a concentration of greater than or equal to 0.1 wt % and less than or equal to 25 wt %. In some embodiments, the PVA concentration is greater than or equal to 0.1 wt %, greater than or equal to 0.5 wt %, greater than or equal to 1 wt %, greater than or equal to 5 wt %, greater than or equal to 10 wt %, greater than or equal to 15 wt %, greater than or equal to 20 wt %, or greater than or equal to 25 wt %. In certain embodiments, the PVA concentration is less than or equal to 25 wt %, less than or equal to 20 wt %, less than or equal to 15 wt %, less than or equal to 10 wt %, less than or equal to 5 wt %, less than or equal to 1 wt %, less than or equal to 0.5 wt %, or less than or equal to 0.1 wt %. Combinations of the above-referenced ranges are also possible (e.g., greater than or equal to 0.1 wt % and less than or equal to 25 wt %).

In some embodiments, the solution coating comprises a UV-curable adhesive (e.g., a UV-curable adhesive capable of reacting with poly(vinyl alcohol)) such that the UV-curable adhesive reduces the equilibrium water content e.g., such that delamination with a hydrophobic bond does not occur when exposed to water, saline, blood, or other physiological fluids.

In some embodiments, the solution coating comprises a cross-linkable adhesive (e.g., to generate covalent cross-linking bonds resistant to hydrolysis) such as via etherification, diurea, thiolcyanates and carbon-based cyanates, and/or using the hydroxyl moiety from poly(vinyl alcohol). Non-limiting examples of suitable (e.g., hydrolysis resistant) cross-linkers include cyanates (e.g., diisocyanates, triisocyanates, diisothiocyanates) and ureas (e.g., isobutylidenediurea, ethylene diurea, propylene diurea), amongst others. Without wishing to be bound by theory, etherification may be achieved under relatively low pH using e.g., sulfuric acid and a mixture of polyethylene glycol, poly(tetrahydrofuran), poly(phenyl ether), amongst others.

In some embodiments, and without wishing to be bound by theory, esterification of a PVA-based solution coating may occur when a multi-carboxylic acid is reacted to bridge two or more PVA molecules together. Esterification is generally prone to hydrolysis (e.g., especially in the presence of basic (high pH) environment). As such, in some embodiments, esterification may provide advantages for the biodegradable release of the adhesive mechanism e.g., to release two non-hydrolyzable devices form each other. In some embodiments, esterification may advantageously be used when a feature is temporarily needed on a PVA-based device such as a sharp tip to insert a device into a vein (e.g., then quickly or slowly breaks down in the vasculature). Non-limiting examples of suitable cross-linkers include oxalic acid, maleic acid, malonic acid, fumaric acid, malic acid, ascorbic acid, succinic acid, adipic acid, glutaric acid, tataric acid, citric acid, poly(acrylic acid), poly(methacrylic acid), poly(caprolactone), poly(lactic acid), and poly(glycolic acid). Without wishing to be bound by theory, a rigid covalent crosslink structure can occur with two or more carboxylic acid groups are connected by a carbon-carbon bonded segment. Such dicarboxylic acid groups may be useful in some embodiments e.g., as useful hydrolyzable cross-link bonds and may be made under low pH conditions.

In some embodiments, the body portion comprises a first material and the component comprises a second material, the same, or different than the first material. In a preferred set of embodiments, the first and/or second materials comprise polymers. Without wishing to be bound by any particular theory, it is generally understood that a bulk polymeric material contains surface polymer chains (herein surface polymers)(e.g., polymer chains within 1 to 100 microns of the air-surface interface) and bulk polymer chains (herein bulk polymers) (e.g., polymer chains more than 100 microns away from the air-surface interface). In some embodiments, the first and/or second materials may comprise pores. Combinations are also possible. For example, first and/or second materials may comprise surface polymers, bulk polymers, and/or pores.

Thus, the surface polymers, bulk polymers, and/or pores of the first material (e.g., body portion) may integrate with the corresponding surface polymers, bulk polymers and/or pores of the second material. The term “integrate” as used herein refers to the polymer chains of the first and/or second material becoming physically entangled with the polymer chains and/or pores of the corresponding material (e.g., the surface polymers of the first material may become entangled with the bulk polymers of the second material). In some cases, the entanglement may further result in one or more non-covalent bonds between the polymer chains of the first and/or second materials. Non-limiting examples include electrostatic interactions (e.g., ionic bonding, hydrogen bonding, halogen bonding), Van der Waals forces (e.g., dipole-dipole interactions, dipole-induced dipole interactions, and London dispersion forces), pi-effects (e.g., pi-pi interactions, cation-pi and anion-pi interactions, and polar-pi interactions), and hydrophobic effects. In certain cases, the presence of one or more non-covalent bonds increases the mechanical properties of the integrated article (e.g., a body part comprising a component).

In some embodiments, the surface polymers interact with at least a portion of the bulk polymers and at least a portion of the pores within the polymer bulk. In certain embodiments, the surface polymers do not interact with at least a portion of the bulk polymers and at least a portion of the pores within the polymer bulk.

In some embodiments, the component (e.g., second water-soluble polymer) is positioned within the bulk of the first material (e.g., first water-soluble polymer). In some embodiments, the component (e.g., second water-soluble polymer) is substantially homogeneously dispersed within the bulk of the first material (e.g., first water-soluble polymer) and the first material is substantially homogenously dispersed within the bulk of the second material. In some embodiments, the component (e.g., second water-soluble polymer) is substantially non-homogeneously dispersed within the bulk of the first material (e.g., first water-soluble polymer) and vice versa.

In some embodiments, the surface polymers of the first material physically integrate with the surface polymers, bulk material, and/or pores of the second material. In some embodiments, the surface polymer of the second material physically integrate with the surface polymers, bulk material, and/or pores of the first material.

In some embodiments, the bulk material of the first material physically integrates with the surface polymers, bulk material, and/or pores of the second material. In some embodiments, the bulk material of the second material physically integrates with the surface polymer, bulk material, and/or pores of the first material.

In some embodiments, the pores of the first material physically integrate with the surface polymers and/or bulk materials of the second material. In some embodiments, the pores of the second material physically integrate with the surface polymers and/or bulk materials of the first material.

In some embodiments, an adhesive may be present. In some embodiments, the adhesive may be physically integrated with the first component and/or the second component.

In some embodiments, an article may comprise a body portion comprising an elongated tube (e.g., catheter shaft). In some cases, the elongated tube may comprise a first material comprising a water-soluble polymer (e.g., PVA). As described elsewhere herein, the water first material is a hydrophilic porous polymeric material that may be processed in either the dehydrated (e.g., tensed) or hydrated state (e.g., relaxed). The elongated tube may further comprise a first portion (e.g. a proximal end) and a second portion (e.g., a distal end), according to certain embodiments. In some embodiments, the first portion comprises a radius of curvature is different, than a radius of curvature of the second portion, when the elongated tube is in a relaxed state (e.g., hydrated and swollen state). In certain embodiments, the first portion comprises a radius of curvature that is greater than a radius of curvature of the second portion, when the elongated tube is in a relaxed state; whereas in certain other embodiments, the first portion comprises a radius of curvature is less than a radius of curvature of the second portion, when the elongated tube is in a relaxed state.

In some embodiments, the radius of curvature of the second portion (e.g., distal end) is greater than or equal to 1 degree, greater than or equal to 2 degrees, greater than or equal to 4 degrees, greater than or equal to 5 degrees, greater than or equal to 10 degrees, greater than or equal to 15 degrees, greater than or equal to 20 degrees, greater than or equal to 40 degrees, greater than or equal to 80 degrees, greater than or equal to 100 degrees, greater than or equal to 120 degrees, greater than or equal to 140 degrees or greater than or equal to 180 degrees relative to the first portion (e.g., proximal end). In certain embodiments, the radius of curvature of the second portion (e.g., distal end) less than or equal to 180 degrees, less than or equal to 140 degrees, less than or equal to 120 degrees, less than or equal to 100 degrees, less than or equal to 80 degrees, less than or equal to 40 degrees, less than or equal to 20 degrees, less than or equal to 15 degrees, less than or equal to 10 degrees, less than or equal to 5 degrees, less than or equal to 4 degrees, less than or equal to 2 degrees, or less than or equal to 1 degree relative to the first portion.

In some embodiments, the invention describes a dual lumen article comprising a first body portion comprising a first material comprising a water-soluble polymer (e.g., PVA). The first body portion may comprise a first lumen (e.g., an elongated tube) having a first inner diameter. In some cases, the first body portion comprises a first portion and a second portion. In some embodiments, the inner diameter of the first portion is greater than, less than, or equal to the inner diameter of the second portion. In a preferred embodiment, the first body portion comprises a first lumen (e.g., elongated tube) of constant dimension (e.g., the inner diameter of the first portion equals the inner diameter of the second portion).

In some embodiments, the dual lumen article further comprises a second body portion comprising a second material, different than the first material, comprising a second lumen (e.g., a second elongated tube) having a second inner diameter. In some cases, the second body portion comprises a first portion and a second portion. In some embodiments, the inner diameter of the first portion is greater than, less than, or equal to the inner diameter of the second portion (e.g., the second body portion may taper toward one end).

In a preferred set of embodiments, the dual lumen article comprises the second body portion positioned inside the first body portion (e.g., a dual lumen configuration). For example, in some embodiments, the first portion of the second body has an outer diameter less than the first inner diameter of the first lumen, and the second portion of the second body has a second inner diameter about equal to the first inner diameter. Stated another way, the dual lumen article comprises a second tube comprising a proximal end of a first diameter and a distal end of a second diameter is placed within a first tube of constant diameter (with a similar dimension as the proximal end of the second tube). Other configurations are also possible.

In some embodiments, the dual lumen article comprises one or more surface features (e.g., barbs, bulges, etc.,) at an interface between the first body portion and the second body portion. Without wishing to be bound by any particular theory, it is generally believed that such surface features may mechanically reinforce the interface between the first body portion and the second body portion. In certain embodiments, the one or more surface features may be positioned on the first body portion and/or the second body portion.

In some embodiments, the article comprises an intravenous catheter. The intravenous catheter, in certain embodiments, comprises a polymeric material comprising a first water-soluble polymer having a plurality of pores. As described elsewhere herein, the preferred polymeric material comprises a first water-soluble polymer comprises poly(vinyl alcohol), which may be processed into a porous material free of covalent crosslinking agents having a water content of less than 5 w/w % and greater than or equal to 0.1 w/w % in a dehydrated state, and configured to swell in an amount greater than or equal to 5 w/w % and less than or equal to 50 w/w % from a dehydrated state to an equilibrium water content state in less than or equal to 60 minutes at 25° C.

In some embodiments, the polymeric material is processed to have a water content of greater than or equal to 0.1 w/w %, greater than or equal to 0.5 w/w %, greater than or equal to 1 w/w %, greater than or equal to 2 w/w %, greater than or equal to 3 w/w %, greater than or equal to 4 w/w %, or greater than or equal to 5 w/w % in the dehydrate state. In certain embodiments, the polymeric material is processed to have a water content of less than or equal to 5 w/w %, less than or equal to 4 w/w %, less than or equal to 3 w/w %, less than or equal to 2 w/w %, less than or equal to 1 w/w %, less than or equal to 0.5 w/w %, or less than or equal to 0.1 w/w %.

In some embodiments, the polymeric material is configured to swell in an amount greater than or equal to 5 w/w %, greater than or equal to 10 w/w %, greater than or equal to 15 w/w %, greater than or equal to 20 w/w %, greater than or equal to 25 w/w %, greater than or equal to 30 w/w %, greater than or equal to 35 w/w %, greater than or equal to 40 w/w %, greater than or equal to 45 w/w %, or greater than or equal to 50 w/w %. In certain embodiments, the polymeric material is configured to swell in an amount less than or equal to 50 w/w %, less than or equal to 45 w/w %, less than or equal to 40 w/w %, less than or equal to 35 w/w %, less than or equal to 30 w/w %, less than or equal to 25 w/w %, less than or equal to 20 w/w %, less than or equal to 15 w/w %, less than or equal to 10 w/w %, or less than or equal to 5 w/w %.

In some embodiments, the polymeric material is configured to swell from a dehydrated state to an equilibrium water content state in less than or equal to 60 minutes, less than or equal to 50 minutes, less than or equal to 40 minutes, less than or equal to 30 minutes, or less than or equal to 15 minutes.

In some embodiments, the intravenous catheter may be configured to be inserted into a subject in the dehydrated, partially hydrated, or hydrated states. In certain embodiments, the catheter may be at least 5%, at least 10%, at least 20%, at least 30%, at least 40%, or at least 50% hydrated prior to inserting the device. In some embodiments, the catheter may be less than or equal to 50%, less than or equal to 40%, less than or equal to 30%, less than or equal to 20%, or less than or equal to 10% hydrated prior to inserting the device. Combinations of the above-referenced ranges are also possible.

In some cases, the intravenous catheter further comprises a lumen and a distal end comprising a tip geometry suitable for intravenous insertion into a subject. The lumen of the intravenous catheter may be any dimension known to those of skill in the art. For example, in some cases, the lumen dimension may be chosen to correspond to standard peripheral intravenous catheter dimensions. Without wishing to be bound to any particular theory, those of skill in the art will know that catheters are commonly sized by gauge (e.g., 14G, 16G, 18G, 20G, 22G, 24G, 26G, and 30G, etc.) with each gauge having a standardized external diameter and length. For example, a standard 14G catheter typically has an external diameter of 2.1 mm whereas a 26G catheter has a 0.6 mm external diameter.

In some embodiments, the intravenous catheter may comprise a tip geometry, for example, to improve insertion into a subject's vein. Any tip geometry known to the skilled artisan may be used. Non-limiting embodiments include a bevel tip needle stylet, bevel tip needle cannula, lancet point needle stylet, back bevel needle stylet, back bevel needle cannula, trocar tip needle stylet, Franseen tip needle cannula, or conical tip needle stylet. In some embodiments, the tip comprises a symmetric geometry; however, in certain embodiments the tip comprises an asymmetrical geometry.

Some aspects of the current disclosure generally relate to catheter systems comprising the catheters described herein and a component configured for administering a therapeutic agent and/or fluid (e.g., saline, Plasma Lyte, etc.,) to a subject and/or withdrawing a bodily fluid from the subject (e.g., blood).

In some embodiments, the catheter system is configured to gain venous access. The system may comprise peripheral devices such as peripheral intravenous lines (e.g., PIVs, for short term access, e.g., up to 96 hours) and/or midline catheters or central devices such as PICCs (e.g., for medium-term access, about 6 months, and especially for antibiotics, TPN, chemotherapy, transfusions, and frequent blood sampling), non-tunneled central catheters (e.g., for short-term access when PIV is not suitable, and especially for resuscitation and central venous pressure monitoring), tunneled central catheters (e.g., for frequent long-term access, and especially for TPN, transfusions, and frequent blood sampling. Can be used with PICC line is contraindicated or not possible), or an implantable port (e.g., for infrequent access on a long-term basis or when lifestyle concerns make one of the other options less appealing).

In some embodiments, the catheter system is configured to gain urinary access. The skilled artisan will understand that current urinary catheter systems include intermittent urinary catheters, indwelling urinary catheters, and suprapubic catheters. Intermittent urinary catheters are the most common and are inserted into the bladder several times a day to drain the bladder and then removed from the body. Indwelling urinary catheters are held inside the bladder by a water filled balloon that prevents it from falling out (commonly referred to as Foley catheters). Urine is drained through a tube connected to a collection bag. In some cases, the indwelling catheter may comprise a valve which can be opened to allow urine to be drained and closed to allow the bladder to fill with urine until drainage is convenient. Suprapubic catheters are left in place for long-term use. Rather than being inserted through the urethra, the catheter is inserted through a hole in the abdomen and directly placed inside the bladder. Suprapubic catheters are typically employed when the urethra is damaged or blocked (e.g., due to cancer). Like Indwelling catheters, suprapubic catheters may contain a valve to permit intermittent drainage.

As such, the systems described herein may comprise any number of components in addition to the catheter itself. Exemplary embodiments include, but are not limited to, a collection bag, drainage tube, drainage port, a catheter cuff, and/or catheter balloon, hub and wings, a valve, injection port cap, needle grip, a flashback chamber, a luer lock plug, and optionally a needle.

The articles of the present invention comprise, in some embodiments, a first material (e.g., a water-soluble polymer, hydrophilic porous material, etc.,). Below is a non-limiting description of the various embodiments contemplated for the first material as described elsewhere herein.

Some aspects of the current disclosure generally relate to an article (e.g., a device) comprising a body portion comprising a first porous material. In some embodiments, the plurality of pores has a particular mean pore size. In some embodiments, the mean pore size of the plurality of pores is less than or equal to 500 nm, less than or equal to 450 nm, less than or equal to 400 nm, less than or equal to 350 nm, less than or equal to 300 nm, less than or equal to 250 nm, less than or equal to 200 nm, less than or equal to 150 nm, less than or equal to 100 nm, less than or equal to 75 nm, less than or equal to 50 nm, less than or equal to 25 nm, less than or equal to 20 nm, or less than or equal to 15 nm. In some embodiments, the plurality of pores have a mean pore size of greater than or equal to 10 nm, greater than or equal to 15 nm, greater than or equal to 20 nm, greater than or equal to 25 nm, greater than or equal to 50 nm, greater than or equal to 75 nm, greater than or equal to 100 nm, greater than or equal to 150 nm, greater than or equal to 200 nm, greater than or equal to 250 nm, greater than or equal to 300 nm, greater than or equal to 350 nm, greater than or equal to 400 nm, or greater than or equal to 450 nm. Combinations of the above referenced ranges are also possible (e.g., less than or equal to 500 nm and greater than or equal to 10 nm). Other ranges are also possible. Mean pore size, as described herein, may be determined by mercury intrusion porosimetry of the material in a first configuration (e.g., a water content less than the equilibrium water content state, such as the dehydrated state). In some embodiments, the pores are interconnected.

In some embodiments, at least a portion of the plurality of pores may be characterized as nanopores, e.g., pores having an average cross-sectional dimension of less than 1 micron. In some embodiments, at least a portion of the plurality of pores may be characterized as micropores, e.g., pores having an average cross-sectional dimension of less than 1 mm and greater than or equal to 1 micron. In some embodiments, at least 50% (e.g., at least 60%, at least 70%, at least 80%, at least 90%, at least 95%, at least 98%, at least 99%, at least 99.9%) of the plurality of pores have a diameter that is less than or equal to 1 micron, less than or equal to 800 nm, less than or equal to 600 nm, less than or equal to 500 nm, less than or equal to 450 nm, less than or equal to 400 nm, less than or equal to 350 nm, less than or equal to 300 nm, less than or equal to 250 nm, less than or equal to 200 nm, less than or equal to 150 nm, less than or equal to 100 nm, less than or equal to 75 nm, less than or equal to 50 nm, less than or equal to 25 nm, less than or equal to 20 nm, or less than or equal to 15 nm. In some cases, at least 50% of the plurality of pores have a diameter that is greater than or equal to 10 nm, greater than or equal to 15 nm, greater than or equal to 20 nm, greater than or equal to 25 nm, greater than or equal to 50 nm, greater than or equal to 75 nm, greater than or equal to 100 nm, greater than or equal to 150 nm, greater than or equal to 200 nm, greater than or equal to 250 nm, greater than or equal to 300 nm, greater than or equal to 350 nm, greater than or equal to 400 nm, greater than or equal to 450 nm, greater than or equal to 500 nm, greater than or equal to 600 nm, or greater than or equal to 800 nm. Combinations of the above referenced ranges are also possible (e.g., less than or equal to 1000 nm and greater than or equal to 10 nm). Other ranges are also possible. In some embodiments, the pores are interconnected.

The first material described herein may have a particular porosity e.g., in a first configuration (e.g., a water content less than the equilibrium water content state, such as the dehydrated state). In some embodiments, the device (first water-soluble polymer) has a porosity of greater than or equal to 5%, greater than or equal to 10%, greater than or equal to 15%, greater than or equal to 20%, greater than or equal to 25%, greater than or equal to 30%, greater than or equal to 35%, greater than or equal to 40%, or greater than or equal to 45% in a first configuration (e.g., a water content less than the equilibrium water content state, such as the dehydrated state). In some embodiments, the device (first water-soluble polymer) has a porosity of less than or equal to 50%, less than or equal to 45%, less than or equal to 40%, less than or equal to 35%, less than or equal to 30%, less than or equal to 25%, less than or equal to 20%, less than or equal to 15%, or less than or equal to 10% in a first configuration (e.g., a water content less than the equilibrium water content state, such as the dehydrated state). Combinations of the above-referenced ranges are also possible (e.g., greater than or equal to 5% and less than or equal to 50% in a first configuration (e.g., a water content less than the equilibrium water content state, such as the dehydrated state)). Other ranges are also possible.

In some embodiments, the first material (e.g., first water-soluble polymer) is hydrophilic. The term “hydrophilic” as used herein is given its ordinary meaning in the art and refers to a material surface having a water contact angle as determined by goniometry of less than 90 degrees. In some embodiments, the polymeric material (or a surface thereof) (e.g., of the device) has a water contact angle of less than or equal to 45 degrees, less than or equal to 40 degrees, less than or equal to 35 degrees, less than or equal to 30 degrees, less than or equal to 25 degrees, less than or equal to 20 degrees, less than or equal to 15 degrees, less than or equal to 10 degrees, less than or equal to 5 degrees, or less than or equal to 2 degrees at an equilibrium water content state. In some embodiments, the polymeric material (or a surface thereof) has a water contact angle of greater than or equal to 1 degree, greater than or equal to 2 degrees, greater than or equal to 5 degrees, greater than or equal to 10 degrees, greater than or equal to 15 degrees, greater than or equal to 20 degrees, greater than or equal to 25 degrees, greater than or equal to 30 degrees, greater than or equal to 35 degrees, or greater than or equal to 40 degrees at an equilibrium water content state. Combinations of the above-referenced ranges are also possible (e.g., greater than or equal to 1 degree and less than or equal to 45 degrees). Other ranges are also possible.

Equilibrium water content state, as used herein, refers the steady state of a device (or material) which does not gain (e.g., absorb) or lose bulk water content as determined when submerged in water at 25° C. without externally applied mechanical stresses. Those skilled in the art would understand that steady state (or equilibrium water content state) shall be understood to not require absolute conformance to a strict thermodynamic definition of such term, but, rather, shall be understood to indicate conformance to the thermodynamic definition of such term to the extent possible for the subject matter so characterized as would be understood by one skilled in the art most closely related to such subject matter (e.g., accounting for factors such as passive diffusion and/or Brownian motion).

In some embodiments, the equilibrium water content state of the first water-soluble polymer is greater than or equal to 10 w/w %, greater than or equal to 20 w/w %, greater than or equal to 25 w/w %, greater than or equal to 30 w/w %, greater than or equal to 35 w/w %, greater than or equal to 40 w/w %, greater than or equal to 45 w/w %, greater than or equal to 50 w/w %, greater than or equal to 55 w/w %, greater than or equal to 60 w/w %, greater than or equal to 65 w/w %, or greater than or equal to 70 w/w %. In some embodiments, the equilibrium water content state of the first water-soluble polymer is less than or equal to 80 w/w %, less than or equal to 75 w/w %, less than or equal to 70 w/w %, less than or equal to 65 w/w %, less than or equal to 60 w/w %, less than or equal to 55 w/w %, less than or equal to 50 w/w %, less than or equal to 45 w/w %, less than or equal to 40 w/w %, less than or equal to 35 w/w %, less than or equal to 30 w/w %, less than or equal to 25 w/w %, or less than or equal to 20 w/w %. Combinations of these ranges are also possible (e.g., greater than or equal to 10 w/w % and less than or equal to 80 w/w %). Other ranges are also possible.

In some embodiments, the first material is substantially lubricious at an equilibrium water content state. For example, in some embodiments, the first material (e.g., first water-soluble polymer) has a surface roughness of less than or equal to 1000 nm (Ra) at an equilibrium water content state. In some embodiments, the first material (e.g., first water-soluble polymer) has a surface roughness (Ra) of less than or equal to 500 nm, less than or equal to 400 nm, less than or equal to 300 nm, less than or equal to 250 nm, less than or equal to 200 nm, less than or equal to 150 nm, less than or equal to 100 nm, less than or equal to 50 nm, less than or equal to 25 nm, less than or equal to 10 nm, or less than or equal to 5 nm at an equilibrium water content state. In some embodiments, the first material (or polymeric material of the device) has a surface roughness (Ra) of greater than or equal to 5 nm at an equilibrium water content state, greater than or equal to 10 nm, greater than or equal to 25 nm, greater than or equal to 50 nm, greater than or equal to 100 nm, greater than or equal to 150 nm, greater than or equal to 200 nm, greater than or equal to 250 nm, greater than or equal to 300 nm, greater than or equal to 400 nm, or greater than or equal to 500 nm at an equilibrium water content state. Combinations of the above-referenced ranges are also possible (e.g., greater than or equal to 5 nm and less than or equal to 1000 nm). Other ranges are also possible.

In some embodiments, the first material has a surface having a coefficient of friction of less than or equal to 0.10 at an equilibrium water content state. For example, the coefficient of friction of a surface of the device (or polymeric material of the device) is less than or equal to 0.1, less than or equal to 0.09, less than or equal to 0.08, less than or equal to 0.07, less than or equal to 0.06, less than or equal to 0.05, less than or equal to 0.04, less than or equal to 0.03, or less than or equal to 0.02. In some embodiments, the coefficient of friction of the surface of the body portion (or polymeric material of the device) is greater than or equal to 0.01, greater than or equal to 0.02, greater than or equal to 0.03, greater than or equal to 0.04, greater than or equal to 0.05, greater than or equal to 0.06, greater than or equal to 0.07, greater than or equal to 0.08, or greater than or equal to 0.09. Combinations of the above-referenced ranges are also possible (e.g., less than or equal to 0.1 and greater than or equal to 0.01). Other ranges are also possible.

Advantageously, the first material (e.g., first water-soluble polymer) described herein may have low sorption of substances such as therapeutic agents (and/or e.g., proteins) in the presence of a dynamic fluid comprising such substances. Such devices and compositions may be useful for use in subjects where, for example, the presence of the device should not substantially decrease the availability and/or concentration of therapeutic agents delivered to the subject (e.g., via the device). In some embodiments, administration of therapeutic agents via a fluid flowed within the devices described herein do not substantially reduce the concentration of the therapeutic agent within the fluid. In some cases, the device may not absorb and/or adsorb the therapeutic agent, e.g., during flow or use.

In some embodiments, less than or equal to 0.5 w/w % sorption of a therapeutic agent to the surface and/or bulk of the first material (e.g., first water-soluble polymer) occurs as determined at equilibrium water content after exposing the polymer to the therapeutic agent and flushing with 5 times the volume of the device with an aqueous solution, such as water or normal saline. In some embodiments, less than or equal to 0.5 w/w %, less than or equal to 0.4 w/w %, less than or equal to 0.3 w/w %, less than or equal to 0.2 w/w %, or less than or equal to 0.1 w/w % sorption of the therapeutic agent to the surface and/or bulk of the first water-soluble polymer occurs. In some embodiments, greater than or equal to 0.05 w/w %, greater than or equal to 0.1 w/w %, greater than or equal to 0.2 w/w %, greater than or equal to 0.3 w/w %, or greater than or equal to 0.4 w/w % sorption of the therapeutic agent to the surface and/or bulk of the first water-soluble polymer occurs. Combinations of the above-referenced ranges are also possible (e.g., less than or equal to 0.5 w/w % and greater than or equal to 0.05 w/w %). Other ranges are also possible.

Advantageously, the first material described herein may have desirable swelling characteristics (e.g., in water, in saline, in a fluidic environment of a subject).

In some embodiments, the first material (e.g., first water-soluble polymer) described herein has a first configuration (e.g., a water content less than the equilibrium water content state, such as the dehydrated state) with a water content of less than or equal to 40 w/w %, less than or equal to 30 w/w %, less than or equal to 20 w/w %, less than or equal to 10 w/w %, less than or equal to 5 w/w %, less than or equal to 4 w/w %, less than or equal to 3 w/w %, less than or equal to 2 w/w %, less than or equal to 1 w/w %, less than or equal to 0.8 w/w %, less than or equal to 0.6 w/w %, less than or equal to 0.4 w/w %, or less than or equal to 0.2 w/w %. In some embodiments, the first material described herein have a first configuration (e.g., a water content less than the equilibrium water content state, such as the dehydrated state) with a water content of greater than or equal to 0.1 w/w %, greater than or equal to 0.2 w/w %, greater than or equal to 0.4 w/w %, greater than or equal to 0.6 w/w %, greater than or equal to 0.8 w/w %, greater than or equal to 1 w/w %, greater than or equal to 2 w/w %, greater than or equal to 3 w/w %, greater than or equal to 4 w/w %, greater than or equal to 5 w/w %, greater than or equal to 6 w/w %, greater than or equal to 7 w/w %, greater than or equal to 8 w/w %, greater than or equal to 9 w/w %, greater than or equal to 10 w/w %, greater than or equal to 15 w/w, greater than or equal to 20 w/w %, greater than or equal to 25 w/w %, greater than or equal to 30 w/w %, or greater than or equal to 35 w/w %. Combinations of the above-referenced ranges are also possible (e.g., greater than or equal to 0.1 w/w % and less than 5 w/w %, greater than or equal to 2 w/w % and less than or equal to 10 w/w %, greater than or equal to 2 w/w % and less than or equal to 40 w/w %, or greater than or equal to 6 w/w % and less than or equal to 40 w/w %). Other ranges are also possible.

In some embodiments, the first material described herein has a first configuration (e.g., a water content less than the equilibrium water content state, such as the dehydrated state). In some embodiments, the first material (or polymeric materials) described herein swell from a first configuration (e.g., a water content less than the equilibrium water content state, such as the dehydrated state) to a second configuration (e.g., the equilibrium water content state) in less than or equal to 60 minutes (e.g., less than or equal to 10 minutes, less than or equal to 5 minutes, less than or equal to 1 minute, or less than or equal to 10 seconds). In some embodiments, the first material (or polymeric materials) described herein swell from a first configuration (e.g., a water content less than the equilibrium water content state, such as the dehydrated state) to a second configuration (e.g., the equilibrium water content state) at 25° C.

In some embodiments, the first material described herein swell in an amount greater than or equal to 2 w/w %, greater than or equal to 3 w/w %, greater than or equal to 4 w/w %, greater than or equal to 5 w/w %, greater than or equal to 10 w/w %, greater than or equal to 15 w/w %, greater than or equal to 20 w/w %, greater than or equal to 25 w/w %, greater than or equal to 30 w/w %, greater than or equal to 35 w/w %, greater than or equal to 40 w/w %, or greater than or equal to 45 w/w %, for example, from a first configuration (e.g., a water content less than the equilibrium water content state, such as the dehydrated state) to a second configuration (e.g., the equilibrium water content state). In some embodiments, the first material (or polymeric materials) described herein swell in an amount less than or equal to 50 w/w %, less than or equal to 45 w/w %, less than or equal to 40 w/w %, less than or equal to 35 w/w %, less than or equal to 30 w/w %, less than or equal to 25 w/w %, less than or equal to 20 w/w %, less than or equal to 15 w/w %, less than or equal to 10 w/w %, less than or equal to 5 w/w %, less than or equal to 4 w/w %, or less than or equal to 3 w/w %, for example, from a first configuration (e.g., a water content less than the equilibrium water content state, such as the dehydrated state) to a second configuration (e.g., the equilibrium water content state). Combinations of these ranges are also possible (e.g., greater than or equal to 5 w/w % and less than or equal to 40 w/w %).

In some embodiments, the first material described herein is in a first configuration (e.g., a water content less than the equilibrium water content state, such as the dehydrated state). For example, in some embodiments, the first material (e.g., the first water-soluble polymer) described herein has a water content of less than or equal to 40 w/w %, less than or equal to 30 w/w %, less than or equal to 20 w/w %, less than or equal to 10 w/w %, less than or equal to 5 w/w %, less than or equal to 4 w/w %, less than or equal to 3 w/w %, less than or equal to 2 w/w %, less than or equal to 1 w/w %, less than or equal to 0.8 w/w %, less than or equal to 0.6 w/w %, less than or equal to 0.4 w/w %, or less than or equal to 0.2 w/w % in a first configuration (e.g., a water content less than the equilibrium water content state, such as the dehydrated state). In some embodiments, the first material (e.g., first water-soluble polymer) described herein has a water content of greater than or equal to 0.1 w/w %, greater than or equal to 0.2 w/w %, greater than or equal to 0.4 w/w %, greater than or equal to 0.6 w/w %, greater than or equal to 0.8 w/w %, greater than or equal to 1 w/w %, greater than or equal to 2 w/w %, greater than or equal to 3 w/w %, or greater than or equal to 4 w/w %. Combinations of the above-referenced ranges are also possible (e.g., greater than or equal to 0.1 w/w % and less than 5 w/w % or greater than or equal to 2 w/w % and less than or equal to 40 w/w %). Other ranges are also possible. The dehydrated state, as described herein, generally refers to the steady state determined under ambient conditions in which the device (first water-soluble polymer) has no appreciable decrease in water content of less than 5 w/w % over 24 hours. In some embodiments, the devices described herein may comprise a coating or unbound porogen, such as a humectant coating, as described in more detail below.

Advantageously, the first material (e.g., first water-soluble polymer) and compositions described herein may be configured for rapid swelling in the presence of an aqueous solution, such as water and/or saline. In some embodiments, the first material is configured to swell in an amount greater than or equal to 2 w/w %, greater than or equal to 5 w/w %, greater than or equal to 10 w/w %, greater than or equal to 15 w/w %, greater than or equal to 20 w/w %, greater than or equal to 25 w/w %, greater than or equal to 30 w/w %, greater than or equal to 35 w/w %, greater than or equal to 40 w/w %, or greater than or equal to 45 w/w %, for example, from a first configuration (e.g., a water content less than the equilibrium water content state, such as the dehydrated state) to a second configuration (e.g., equilibrium water content state), e.g., at 25° C., e.g., in a particular amount of time (e.g., less than or equal to 60 minutes, less than or equal to 10 minutes, less than or equal to 5 minutes, less than or equal to 1 minute, or less than or equal to 10 seconds), as described in more detail below. In some embodiments, the first material configured to swell in an amount less than or equal to 50 w/w %, less than or equal to 45 w/w %, less than or equal to 40 w/w %, less than or equal to 35 w/w %, less than or equal to 30 w/w %, less than or equal to 25 w/w %, less than or equal to 20 w/w %, less than or equal to 15 w/w %, or less than or equal to 10 w/w %, for example, from a first configuration (e.g., a water content less than the equilibrium water content state, such as the dehydrated state) to a second configuration (e.g., an equilibrium water content state), e.g., at 25° C., e.g., in a particular amount of time (e.g., less than or equal to 60 minutes, less than or equal to 10 minutes, less than or equal to 5 minutes, less than or equal to 1 minute, or less than or equal to 10 seconds) as described in more detail below. Combinations of the above-referenced ranges are also possible (e.g., greater than or equal to 5 w/w % and less than or equal to 50 w/w %). Other ranges are also possible.

In an exemplary embodiment, the first material is configured to swell to an equilibrium water content state (e.g., greater than or equal to 5 w/w % or greater than or equal to 20 w/w % and less than or equal to 80 w/w %) in less than or equal to 60 minutes (e.g., less than or equal to 10 minutes, less than or equal to 5 minutes, less than or equal to 1 minute, or less than or equal to 10 seconds) from a first configuration (e.g., a water content less than the equilibrium water content state, such as the dehydrated state) (e.g., less than 5 w/w % or greater than or equal to 2 w/w % and less than or equal to 40 w/w %) in water. In some embodiments, the first material is configured to swell to an equilibrium water content (e.g., greater than or equal to 5 w/w % or greater than or equal to 20 w/w % and less than or equal to 80 w/w %) in less than or equal to 60 minutes (e.g., less than or equal to 10 minutes, less than or equal to 5 minutes, less than or equal to 1 minute, or less than or equal to 10 seconds) from, for example, a first configuration (e.g., a water content less than the equilibrium water content state, such as the dehydrated state) (e.g., less than 5 w/w %) in standard normal saline. In another exemplary embodiment, the first material (e.g., first water-soluble polymer) is configured to swell to an equilibrium water content (e.g., greater than or equal to 5 w/w % or greater than or equal to 20 w/w % and less than or equal to 80 w/w %) in less than or equal to 60 minutes (e.g., less than or equal to 10 minutes, less than or equal to 5 minutes, less than or equal to 1 minute, or less than or equal to 10 seconds) from, for example, a first configuration (e.g., a water content less than the equilibrium water content state, such as the dehydrated state) (e.g., less than 5 w/w %) in normal saline.

In some embodiments, the first material has a particular length in the first configuration (e.g., a water content less than the equilibrium water content state, such as the dehydrated state). In some embodiments, the device (first water-soluble polymer) has an increase in overall length in the equilibrium water content state of greater than or equal to 0.1%, greater than or equal to 0.5%, greater than or equal to 1%, greater than or equal to 2%, greater than or equal to 4%, greater than or equal to 6%, greater than or equal to 8%, greater than or equal to 10%, greater than or equal to 12%, greater than or equal to 14%, greater than or equal to 16%, or greater than or equal to 18% as compared to its length in a first configuration (e.g., a water content less than the equilibrium water content state, such as the dehydrated state). In some cases, the first material (first water-soluble polymer) has an increase in overall length in the equilibrium water content state of less than or equal to 20%, less than or equal to 18%, less than or equal to 16%, less than or equal to 14%, less than or equal to 12%, less than or equal to 10%, less than or equal to 8%, less than or equal to 6%, less than or equal to 4%, less than or equal to 2%, less than or equal to 1%, or less than or equal to 0.5% as compared to its length in the first configuration (e.g., a water content less than the equilibrium water content state, such as the dehydrated state). Combinations of the above-referenced ranges are also possible (e.g., greater than or equal to 0.1% and less than or equal to 20%). Other ranges are also possible.

In some embodiments, the first material has a particular outer maximum cross-sectional dimension, such as an outer diameter of a cylindrical tube, an oval tube, an oblong tube, or square tube. In embodiments where the device comprises multiple lumens, the outer diameter refers to the outer maximum cross-sectional dimension of one or more of the lumens. For example, in some embodiments only one lumen may have the recited outer diameter. In other embodiments, each and every lumen may independently have the recited outer diameter. In some embodiments, the first material (e.g., first water-soluble polymer) has an increase in an outer maximum cross-sectional dimension (e.g., outer diameter) in the equilibrium water content state of greater than or equal to 0.1%, greater than or equal to 0.5%, greater than or equal to 1%, greater than or equal to 2%, greater than or equal to 4%, greater than or equal to 6%, greater than or equal to 8%, greater than or equal to 10%, greater than or equal to 12%, greater than or equal to 14%, greater than or equal to 16%, or greater than or equal to 18% as compared to the maximum cross-sectional dimension (e.g., outer diameter) in a first configuration (e.g., a water content less than the equilibrium water content state, such as the dehydrated state). In some cases, the first material (e.g., first water-soluble polymer) has an increase in the maximum cross-sectional dimension (e.g., outer diameter) in the equilibrium water content state of less than or equal to 20%, less than or equal to 18%, less than or equal to 16%, less than or equal to 14%, less than or equal to 12%, less than or equal to 10%, less than or equal to 8%, less than or equal to 6%, less than or equal to 4%, less than or equal to 2%, less than or equal to 1%, or less than or equal to 0.5% as compared to the maximum cross-sectional dimension (e.g., outer diameter) in a first configuration (e.g., a water content less than the equilibrium water content state, such as the dehydrated state). Combinations of the above-referenced ranges are also possible (e.g., greater than or equal to 0.1% and less than or equal to 20%, greater than or equal to 0.1% and less than or equal to 10%). Other ranges are also possible.

In some embodiments, the first material (e.g., first water-soluble polymer) has a particular inner diameter (e.g., in an embodiment in which the device comprises a hollow core), which is the maximum inner cross-sectional dimension, such as the inner diameter of a cylindrical tube or square tube (or other non-circular device or body portion). In embodiments where the first material (e.g., first water-soluble polymer) comprises multiple lumens, the inner diameter refers to the maximum inner cross-sectional dimension (i.e., the maximum inner cross-sectional dimension of the largest lumen). In some embodiments, the first material (e.g., first water-soluble polymer) has an increase in the inner diameter in the equilibrium water content state of greater than or equal to 0.1%, greater than or equal to 0.5%, greater than or equal to 1%, greater than or equal to 2%, greater than or equal to 4%, greater than or equal to 6%, greater than or equal to 8%, greater than or equal to 10%, greater than or equal to 12%, greater than or equal to 14%, greater than or equal to 16%, or greater than or equal to 18% as compared to the inner diameter in a first configuration (e.g., a water content less than the equilibrium water content state, such as the dehydrated state). In some cases, the first material (e.g., first water-soluble polymer) has an increase in the inner diameter in the equilibrium water content state of less than or equal to 20%, less than or equal to 18%, less than or equal to 16%, less than or equal to 14%, less than or equal to 12%, less than or equal to 10%, less than or equal to 8%, less than or equal to 6%, less than or equal to 4%, less than or equal to 2%, less than or equal to 1%, or less than or equal to 0.5% as compared to the inner diameter in a first configuration (e.g., a water content less than the equilibrium water content state, such as the dehydrated state). Combinations of the above-referenced ranges are also possible (e.g., greater than or equal to 0.1% and less than or equal to 20%). Other ranges are also possible.

In some embodiments, the first material (e.g., first water-soluble polymer) has a larger percentage increase in the overall length than an increase in inner diameter and/or outer diameter when the first material (e.g., first water-soluble polymer) swells from a first configuration (e.g., a water content less than the equilibrium water content state, such as the dehydrated state) to a second configuration (e.g., the equilibrium water content state). For example, in some embodiments, the overall length may increase by 1-20% (e.g., 5-15%) while the inner diameter and/or outer diameter increases by 0.1-19% (e.g., 1-10%).

In some embodiments, the ratio of the percentage increase in the overall length to the percentage increase in the inner diameter and/or outer diameter when the first material (e.g., first water-soluble polymer) swells from a first configuration (e.g., a water content less than the equilibrium water content state, such as the dehydrated state) to a second configuration (e.g., the equilibrium water content state) is greater than or equal to 1.1, greater than or equal to 1.5, greater than or equal to 2, greater than or equal to 5, greater than or equal to 7, or greater than or equal to 10. In some embodiments, the ratio of the percentage increase in the overall length to the percentage increase in the inner diameter and/or outer diameter when the first material (e.g., first water-soluble polymer) swells from a first configuration (e.g., a water content less than the equilibrium water content state, such as the dehydrated state) to a second configuration (e.g., the equilibrium water content state) is less than or equal to 20, less than or equal to 15, less than or equal to 10, less than or equal to 5, or less than or equal to 2. Combinations of these ranges are also possible (e.g., 1.1-20).

In some embodiments, the first material (e.g., first water-soluble polymer) has a larger percentage increase in the inner diameter and/or outer diameter than in overall length when the first material (e.g., first water-soluble polymer) swells from a first configuration (e.g., a water content less than the equilibrium water content state, such as the dehydrated state).

In some embodiments, the ratio of the percentage increase in the inner diameter and/or outer diameter to the percentage increase in the overall length when the first material (e.g., first water-soluble polymer) swells from a first configuration (e.g., a water content less than the equilibrium water content state, such as the dehydrated state) to a second configuration (e.g., the equilibrium water content state) is greater than or equal to 1.1, greater than or equal to 1.5, greater than or equal to 2, greater than or equal to 5, greater than or equal to 7, or greater than or equal to 10. In some embodiments, the ratio of the percentage increase in the inner diameter and/or outer diameter to the percentage increase in the overall length when the first material (e.g., first water-soluble polymer) swells from a first configuration (e.g., a water content less than the equilibrium water content state, such as the dehydrated state) to a second configuration (e.g., the equilibrium water content state) is less than or equal to 20, less than or equal to 10, less than or equal to 5, or less than or equal to 2. Combinations of these ranges are also possible (e.g., 1.1-20).

In some embodiments, the first material comprises one or more polymers having desirable mechanical properties. For example, in some embodiments, the first material has a Young's elastic modulus in the first configuration (e.g., a water content less than the equilibrium water content state, such as the dehydrated state) (e.g., less than 5 w/w % water content) of greater than or equal to 100 MPa, greater than or equal to 250 MPa, greater than or equal to 500 MPa, greater than or equal to 600 MPa, greater than or equal to 750 MPa, greater than or equal to 800 MPa, greater than or equal to 900 MPa, greater than or equal to 1000 MPa, greater than or equal to 1250 MPa, greater than or equal to 1500 MPa, greater than or equal to 1750 MPa, greater than or equal to 2000 MPa, greater than or equal to 2500 MPa, greater than or equal to 3000 MPa, greater than or equal to 3500 MPa, or greater than or equal to 4000 MPa. In some embodiments, the first and/or second material has a Young's elastic modulus in the first configuration (e.g., a water content less than the equilibrium water content state, such as the dehydrated state) (e.g., less than 5 w/w % water content) of less than or equal to 5000 MPa, less than or equal to 4000 MPa, less than or equal to 3500 MPa, less than or equal to 3000 MPa, less than or equal to 2500 MPa, less than or equal to 2000 MPa, less than or equal to 1750 MPa, less than or equal to 1500 MPa, less than or equal to 1250 MPa, less than or equal to 1000 MPa, less than or equal to 900 MPa, less than or equal to 800 MPa, less than or equal to 750 MPa, less than or equal to 600 MPa, less than or equal to 500 MPa, or less than or equal to 250 MPa. Combinations of the above-referenced ranges are also possible (e.g., greater than or equal to 100 MPa and less than or equal to 5000 MPa). Other ranges are also possible.

In some embodiments, the first material has a Young's elastic modulus at an equilibrium water content state of less than or equal to 300 MPa, less than or equal to 250 MPa, less than or equal to 200 MPa, less than or equal to 150 MPa, less than or equal to 100 MPa, less than or equal to 75 MPa, less than or equal to 50 MPa, less than or equal to 25 MPa, less than or equal to 20 MPa, or less than or equal to 10 MPa. In some embodiments, the first material has a Young's elastic modulus at an equilibrium water content state of greater than or equal to 5 MPa, greater than or equal to 10 MPa, greater than or equal to 20 MPa, greater than or equal to 25 MPa, greater than or equal to 50 MPa, greater than or equal to 75 MPa, greater than or equal to 100 MPa, greater than or equal to 150 MPa, greater than or equal to 200 MPa, or greater than or equal to 250 MPa. Combinations of the above-referenced ranges are also possible (e.g., less than or equal to 300 MPa and greater than or equal to 5 MPa). Other ranges are also possible.

In a preferred set of embodiments, the first material has a Young's elastic modulus of greater than or equal to 500 MPa, greater than or equal to 600 MPa, greater than or equal to 700 MPa, greater than or equal to 800 MPa, greater than or equal to 900 MPa, or greater than or equal to 1000 MPa in the dehydrated state. In certain embodiments, the first material has a Young's elastic modulus of less than or equal to 1000 MPa, less than or equal to 900 MPa, less than or equal to 800 MPa, less than or equal to 700 MPa, less than or equal to 600 MPa, or less than or equal to 500 MPa in the dehydrated state.

In another preferred set of embodiments, the first material has a Young's elastic modulus of less than or equal to 300 MPa and greater than or equal to 5 MPa at an equilibrium water content state. In some cases, the first material may have a Young's elastic modulus of greater than or equal to 500 MPa in a dehydrated state and a Young's elastic modulus of less than or equal to 300 MPa and greater than or equal to 5 MPa at an equilibrium water content state.

In some embodiments, the polymeric material has a Young's elastic modulus of less than or equal to 100 MPa and greater than or equal to 5 MPa at an equilibrium water content state.

In a preferred set of embodiments, the devices/articles (e.g., intravascular catheters) described herein comprise the mechanical properties (e.g., stiffness) to support insertion via percutaneous injection into a subject's vein (e.g., to prevent buckling or lumen collapse) or intended used (e.g., withdrawing blood from a subject). Additionally, or alternatively, the devices/articles acquire the appropriate softness following insertion to permit longer dwell times.

In some embodiments, the composition (e.g., comprising or formed of a polymeric material) of the first material (e.g., first water-soluble polymeric material) does not comprise covalent crosslinking, as described in more detail below. In other embodiments, however, the composition (e.g., the first material) comprises physical crosslinking (e.g., interpenetrating network, chain entanglement, and/or one or more bonds such as covalent, ionic, and/or hydrogen bonding). In a particular set of embodiments, no covalent crosslinking agents are used to form the body portion (e.g., the first material and/or second material).

The first material may be present in the device in any suitable amount. For example, in some embodiments, the first material (e.g., first water-soluble polymer) is present in the device and/or body portion in an amount of greater than or equal to 20 w/w %, greater than or equal to 25 w/w %, greater than or equal to 30 w/w %, greater than or equal to 35 w/w %, greater than or equal to 40 w/w %, greater than or equal to 45 w/w %, greater than or equal to 50 w/w %, greater than or equal to 55 w/w %, greater than or equal to 60 w/w %, greater than or equal to 65 w/w %, greater than or equal to 70 w/w %, greater than or equal to 75 w/w %, greater than or equal to 80 w/w %, greater than or equal to 85 w/w %, or greater than or equal to 90 w/w % at an equilibrium water content state. In some embodiments, the first and/or second material (e.g., first and/or second water-soluble polymers) are present in the device and/or body portion in an amount of less than or equal to 95 w/w %, less than or equal to 90 w/w %, less than or equal to 85 w/w %, less than or equal to 80 w/w %, less than or equal to 75 w/w %, less than or equal to 70 w/w %, less than or equal to 65 w/w %, less than or equal to 60 w/w %, less than or equal to 55 w/w %, less than or equal to 50 w/w %, less than or equal to 45 w/w %, less than or equal to 40 w/w %, less than or equal to 35 w/w %, less than or equal to 30 w/w %, or less than or equal to 25 w/w % at an equilibrium water content state. Combinations of the above-referenced ranges are also possible (e.g., greater than or equal to 20 w/w % and less than or equal to 95 w/w %). Other ranges are also possible.

In some embodiments, the first material (e.g., water-soluble polymeric materials) comprises or is selected from the group consisting of poly(vinyl alcohol), poly(acrylic acid), polyethylene glycol, poly(vinyl pyrrolidone), poly(methacrylic sulfobetaine), poly(acrylic sulfobetaine), poly(methacrylic carboxybetaine), poly(acrylic carboxybetaine), povidone, polyacrylamide, poly(N-(2-hydroxypropyl)methacrylamide), polyoxazolines, polyphosphates, polyphosphazenes, polyvinyl acetate, polypropylene glycol, poly(N-isopropylacrylamide), poly(2-hydroxymethylmethacrylate), and combinations thereof. In an exemplary set of embodiments, the first material is poly(vinyl alcohol).

In some embodiments, the first material (e.g., first water-soluble polymeric material) comprises a mixture comprising the first water-soluble polymer and another (e.g., a third) water-soluble polymer. In some embodiments, the third water-soluble polymer comprises or is selected from the group consisting of poly(vinyl alcohol), poly(acrylic acid), polyethylene glycol, poly(vinyl pyrrolidone), poly(methacrylic sulfobetaine), poly(acrylic sulfobetaine), poly(methacrylic carboxybetaine), poly(acrylic carboxybetaine), povidone, polyacrylamide, poly(N-(2-hydroxypropyl)methacrylamide), polyoxazolines, polyphosphates, polyphosphazenes, polyvinyl acetate, polypropylene glycol, poly(N-isopropylacrylamide), poly(2-hydroxymethylmethacrylate), and combinations thereof. The first and other (e.g., third) water-soluble polymers may have different chemical compositions.

In some embodiments, the total weight of the first material (e.g., first and/or third polymers) in the device is greater than or equal to 20 w/w %, greater than or equal to 25 w/w %, greater than or equal to 30 w/w %, greater than or equal to 35 w/w %, greater than or equal to 40 w/w %, greater than or equal to 45 w/w %, greater than or equal to 50 w/w %, greater than or equal to 55 w/w %, greater than or equal to 60 w/w %, greater than or equal to 65 w/w %, greater than or equal to 70 w/w %, greater than or equal to 75 w/w %, greater than or equal to 80 w/w %, greater than or equal to 85 w/w %, greater than or equal to 90 w/w %, greater than or equal to 95 w/w %, greater than or equal to 98 w/w %, or greater than or equal to 99 w/w % at an equilibrium water content state. In some embodiments, the total weight of the first water-soluble polymer and another (e.g., a third) water-soluble polymer in the device in an amount of less than or equal to 100 w/w %, less than or equal to 90 w/w %, less than or equal to 98 w/w %, less than or equal to 95 w/w %, less than or equal to 90 w/w %, less than or equal to 85 w/w %, less than or equal to 80 w/w %, less than or equal to 75 w/w %, less than or equal to 70 w/w %, less than or equal to 65 w/w %, less than or equal to 60 w/w %, less than or equal to 55 w/w %, less than or equal to 50 w/w %, less than or equal to 45 w/w %, less than or equal to 40 w/w %, less than or equal to 35 w/w %, less than or equal to 30 w/w %, or less than or equal to 25 w/w % at an equilibrium water content state. Combinations of the above-referenced ranges are also possible (e.g., greater than or equal to 20 w/w % and less than or equal to 100 w/w %). Other ranges are also possible.

In some embodiments, the ratio of the first water-soluble polymer to the third water-soluble polymer present in the device is less than or equal to 100:0, less than or equal to 99:1, less than or equal to 95:5, less than or equal to 90:10, less than or equal to 80:20, less than or equal to 70:30, less than or equal to 60:40, or less than or equal to 55:45. In some embodiments, the ratio of the first water-soluble polymer to the third water-soluble polymer present in the device is greater than or equal to 50:50, greater than or equal to 60:40, greater than or equal to 70:30, greater than or equal to 80:20, greater than or equal to 90:10, greater than or equal to 95:5, or greater than or equal to 99:1. Combinations of the above-referenced ranges are also possible (e.g., less than or equal to 100:0 and greater than or equal to 50:50). Other ranges are also possible.

In some embodiments, the water-soluble polymer (e.g., the first water-soluble polymer, the second water-soluble polymer, the third water-soluble polymer) has a particular molecular weight. In some embodiments, the molecular weight of the water-soluble polymer (e.g., each, independently, the first water-soluble polymer, the second water-soluble polymer, or the third water-soluble polymer) may be greater than or equal to 40 kDa, greater than or equal to 50 kDa, greater than or equal to 75 kDa, greater than or equal to 100 kDa, greater than or equal to 125 kDa, greater than or equal to 150 kDa, greater than or equal to 175 kDa, greater than or equal to 200 kDa, greater than or equal to 250 kDa, greater than or equal to 300 kDa, greater than or equal to 350 kDa, greater than or equal to 400 kDa, greater than or equal to 450 kDa, greater than or equal to 500 kDa, greater than or equal to 600 kDa, greater than or equal to 700 kDa, greater than or equal to 800 kDa, greater than or equal to 900 kDa, greater than or equal to 1000 kDa, greater than or equal to 1500 kDa, greater than or equal to 2000 kDa, greater than or equal to 3000 kDa, or greater than or equal to 4000 kDa. In some embodiments, the molecular weight of the water-soluble polymer (e.g., each, independently, the first water-soluble polymer, the second water-soluble polymer, or the third water-soluble polymer) may be less than or equal to 5000 kDa, less than or equal to 4000 kDa, less than or equal to 3000 kDa, less than or equal to 2000 kDa, less than or equal to 1500 kDa, less than or equal to 1000 kDa, less than or equal to 900 kDa, less than or equal to 800 kDa, less than or equal to 700 kDa, less than or equal to 600 kDa, less than or equal to 500 kDa, less than or equal to 450 kDa, less than or equal to 400 kDa, less than or equal to 350 kDa, less than or equal to 300 kDa, less than or equal to 250 kDa, less than or equal to 200 kDa, less than or equal to 175 kDa, less than or equal to 150 kDa, less than or equal to 125 kDa, less than or equal to 100 kDa, less than or equal to 75 kDa, or less than or equal to 50 kDa. Combinations of the above-referenced ranges are also possible (e.g., a molecular weight of greater than or equal to 40 kDa and less than or equal to 5000 kDa). Other ranges are also possible.

In some embodiments, the body portion may comprise a first material, a second material, a third material, a fourth material, and so on. The first material, second material, third material, fourth material, etc., may be comprised of the same, or different, materials. For example, in some embodiments, a second body portion comprising a second material (e.g., poly(vinyl alcohol) may be positioned inside a lumen of a first body part comprising a first material (e.g., poly(vinyl alcohol). In certain cases, however, the first component and second component may comprise a different material. In some embodiments, the first material, second material, third material, fourth material, etc., may comprise a water-soluble polymer. The water-soluble polymer may, in some embodiments, be a first water-soluble polymer, a second-water-soluble polymer, a third-water-soluble polymer, a fourth water-soluble polymer, etc. Combinations of the above referenced ranges are also possible (e.g., a first material comprising a fourth water-soluble polymer or a second material comprising a first water-soluble polymer).

As described above and herein, in some embodiments, the body portion comprises a component comprising a second material physically integrated with the first material of the body portion. In some embodiments, the second material comprises a water-soluble polymer that is the same or different as the first water-soluble polymer (e.g., a third water-soluble polymer). In some embodiments, the second material comprises or is selected from the group consisting of poly(vinyl alcohol), poly(acrylic acid), polyethylene glycol, poly(vinyl pyrrolidone), poly(methacrylic sulfobetaine), poly(acrylic sulfobetaine), poly(methacrylic carboxybetaine), poly(acrylic carboxybetaine), povidone polyacrylamide, poly(N-(2-hydroxypropyl)methacrylamide), polyoxazolines, polyphosphates, polyphosphazenes, polyvinyl acetate, polypropylene glycol, poly(N-isopropylacrylamide), poly(2-hydroxymethylmethacrylate), and combinations thereof. In some embodiments, the second material is poly(acrylic acid); in a preferred set of embodiments, the second material is poly(vinyl alcohol). The second material may have a different chemical composition from that of the first water-soluble polymer.

In some embodiments, the second material comprises a water-soluble polymer positioned within at least a portion (e.g., at least 10%, at least 20%, at least 30%, at least 40%, at least 50%, at least 60%, at least 70%, at least 80%, at least 90%, at least 95%, at least 98%, at least 99%, at least 99.99%) of the plurality of pores (e.g., of the first material). In some embodiments, the second material (e.g., third water-soluble polymer) is positioned within less than or equal to 100%, less than or equal to 90%, less than or equal to 80%, less than or equal to 70%, less than or equal to 60%, less than or equal to 50%, less than or equal to 40%, less than or equal to 30%, less than or equal to 20%, or less than or equal to 10% of the plurality of pores of the first material (e.g., at least 10% and less than or equal to 100% of the plurality of pores). Combinations of the above-referenced ranges are also possible.

In some embodiments, the component comprises a second material comprising a porous non-water-soluble polymer. In some embodiments, the second material comprises or is selected from the group consisting of cellulose, cellulose-based materials, nylon, polyethylene terephthalate (e.g., Dacron), poly(1,4-butylene terephthalate), and polyurethane. In certain embodiments, the component is comprised of woven textiles like Dacron® (Poly ethylene terephthalate aka polyester), clectrospun PVA, cotton, wool, Polypropylene (atactic, syndiotactic, isotactic), polyethylene (LLDPE, LDPE, HDPE), cellulose, modified cellulose, hydroxyapatite, and combinations thereof.

The non-water-soluble polymer may be processed into any porous structure using any technique known to those of skill in the art. Exemplary embodiments include structures such as cellular foam cavities, fibers, meshes, netting. For instance, in some embodiments, the second polymeric material may be processed into yarns and fabrics to create complex three-dimensional shapes (e.g., tubular geometries with tapered angles, etc.). In some embodiments, the second material may be a woven structure, in which two sets of polymer yarns are interlaced at right angles; in other embodiments, the second material may be a knit structure, in which loops of polymer yarn are intermeshed; and in some cases, the second material may be braided, in which three or more polymer yarns cross one another in a diagonal pattern, according to other embodiments.

The second material (e.g., third water-soluble polymer) may be present in the device in any suitable amount. For example, in some embodiments, the second material is present in the device in an amount of greater than or equal to 0.05 w/w %, greater than or equal to 0.1 w/w %, greater or than or equal to 0.2 w/w %, greater than or equal to 0.5 w/w %, greater than or equal to 1.0 w/w %, greater than or equal to 2.0 w/w %, greater than or equal to 3.0 w/w %, greater than or equal to 4.0 w/w %, greater than or equal to 5.0 w/w %, greater than or equal to 10 w/w %, greater than or equal to 20 w/w %, greater than or equal to 30 w/w %, greater than or equal to 40 w/w %, greater than or equal to 50 w/w %, greater than or equal to 60 w/w %, greater than or equal to 70 w/w %, greater than or equal to 80 w/w %, or greater than or equal to 90 w/w % at an equilibrium water content state. In some embodiments, the second material is present in the device in an amount of less than or equal to 95 w/w %, less than or equal to 90 w/w %, less than or equal to 80 w/w %, less than or equal to 70 w/w %, less than or equal to 60 w/w %, less than or equal to 50 w/w %, less than or equal to 40 w/w %, less than or equal to 30 w/w %, less than or equal to 20 w/w %, less than or equal to 10 w/w %, less than or equal to 5.0 w/w %, less than or equal to 4.0 w/w %, less than or equal to 3.0 w/w %, less than or equal to 2.0 w/w %, less than or equal to 1.0 w/w %, less than 0.5 w/w %, less than 0.2 w/w %, or less than 0.1 w/w % at an equilibrium water content state. Combinations of the above-referenced ranges are also possible (e.g., greater than or equal to 0.05 w/w % and less than or equal to 95 w/w %). Other ranges are also possible.

In some embodiments, the component is a preformed device, such as a preformed medical device. In some embodiments, the component is a catheter cuff.

In some embodiments, the component may comprise a material that is gas impermeable, for example to air. For example, in some cases, the material may be gas impermeable to oxygen, nitrogen, carbon dioxide, air, or any combination thereof.

In some embodiments, the component may comprise a first material, a second material, a third material, a fourth material, and so on. The first material, second material, third material, fourth material, etc., may be comprised of the same, or different, materials. For example, in some embodiments, a first component comprising a second material and a second component comprising a third material may be bonded to a body portion comprising a first material. In certain cases, however, the first component and second component may comprise the same material (e.g., a second material). In some embodiments, the first material, second material, third material, fourth material, etc., may comprise a water-soluble polymer. The water-soluble polymer may, in some embodiments, be a first water-soluble polymer, a second-water-soluble polymer, a third-water-soluble polymer, a fourth water-soluble polymer, etc. Combinations of the above referenced ranges are also possible (e.g., a first material comprising a fourth water-soluble polymer or a second material comprising a first water-soluble polymer).

Some aspects of the current disclosure generally relate to methods of integrating one or more components with a body portion. In some embodiments, the body portion comprising a polymer is softened and placed into contact with the one or more components under an applied pressure. Without wishing to be bound by any particular theory, it is generally believed that softening the body portion, softens the polymers, which in the presence of an applied pressure, favors polymer chain interdiffusion at the bonding junction, thus forming an integrated article.

In some embodiments, the body portion comprising a polymer is softened during extrusion of the article. Extrusion processes for making the articles of the present invention are described in International Patent Application Publication Nos. WO2018/237166 and WO2017/112878, each of which are hereby incorporated by reference in their entirety. A detailed description of the extrusion processes is also described elsewhere herein. Briefly, however, in some embodiments, the method of forming an integrated article comprises a polymeric mixture comprising at least one water-soluble polymer and a solvent. The concentration of the polymeric mixture is at least 10% w/w, according to some embodiments. For example, in some cases, the concentration of the polymeric mixture is at least 10% w/w, at least 15% w/w, at least 20% w/w, at least 25% w/w, at least 30% w/w, at least 35% w/w, at least 40% w/w, at least 45% w/w, or at least 50% w/w

In some embodiments the solvent may be a homogenous solvent, such as water, or a heterogenous mixture, such as a water/DMSO mixture. In a preferred set of embodiments, the solvent is water. In certain embodiments, the solvent is a co-solvent (e.g., contains two solvents). The skilled artisan will understand that co-solvent systems must comprise solvents that are miscible, or at least partially miscible, with each other. For example, in some embodiments, the co-solvent system comprises a first solvent (e.g., water) and a second solvent (e.g., DMSO). The relative ratio of the first solvent to the second solvent is about 0:1, 0.1:0.9, 0.2:0.8, 0.3:0.7, 0.4:0.6, 0.5:0.5, 0.6:0.4, 0.7:0.3, 0.8:0.2, 0.9:0.1, 1:0.

In some embodiments, the method comprises heating the polymeric mixture to a temperature above the melting point of the polymeric mixture and extruding the polymeric mixture as a body portion. In some embodiments, the method comprises heating the polymeric mixture to at least 10 degrees, at least 15 degrees, at least 20 degrees, at least 30 degrees, at least 40 degrees, at least 50 degrees, at least 60 degrees, at least 70 degrees, at least 80 degrees, at least 90 degrees, or at least 100 degrees above the melting point of the polymeric mixture.

Following extrusion, a component comprising a second material, different than the first material (e.g., a catheter cuff), may be placed in contact with the softened body portion under an externally applied force to facilitate polymer chain interdiffusion at the bonding junction, thus forming an integrated article.

In some embodiments, the solvent (or co-solvent) is removed from the extruded body portion at a temperature above, or below, the freezing point of the solvent. In some preferred embodiments, the solvent is removed from the extruded body portion at a temperature above the freezing point of the solvent. Removal of the solvent, in some cases, may produce a plurality of pores in the extruded body portion. The latter is especially true in the situation where a co-solvent comprises at least one non-solvent for the water-soluble polymer. Without wishing to be bound by any particular theory, it is known in the art that removal of the solvent from a polymer mixture comprising a solvent and a non-solvent (e.g., water/DMF solution containing PVA) drives phase separation into a non-solvent rich phase and a solvent/polymer rich phase. The rate of evaporation can, in some embodiments, control the porous structure. For example, in some embodiments, slow solvent extraction favors coalescence of the non-solvent phase within the solvent/polymer rich phase, resulting in larger pore. On the other hand, rapid solvent extraction inhibits coalescence of the non-solvent phase resulting in smaller pores.

In some embodiments, the solvent is evaporated using heat, such as from a furnace or oven. However, other methods of evaporating the solvent are also contemplated. For example, in some embodiments, the extruded body portion is exposed to a one or more solvent baths containing a non-solvent for the polymer. In other embodiments, the solvent may be removed by placing the extruded body portion into to a stream of inert gas (e.g., argon, nitrogen, etc.). Combinations are also possible. For example, an extruded body portion may be heated, exposed to a solvent bath, and/or exposed to a stream of inert gas.

It is believed that solvent bonding techniques are advantageous to other bonding methods (e.g., plastic welding, adhesive bonding, etc.,) because the bonding may occur below the glass transition temperature of the polymer, although this is not required. Any method and/or technique known to a person of skill in the art may be used to heat the materials described herein. For example, in some embodiments, material heating for bond formation can be done with a split die bonder, RF welder, or hot air station. Depending on the level of hydration of the body portion (e.g., poly(vinyl alcohol)), as well as the thickness of the materials being bonded, the temperature and process time required to reflow the body portion for bond formation may change. For example, process temperatures can range from greater than or equal to 60° C. and less than or equal to 95° and process times can range from greater than or equal to 15 seconds less than or equal to 5 minutes based on these factors. In some embodiments, the process temperature may be greater than or equal to 60° C., greater than or equal to 65° C., greater than or equal to 70° C., greater than or equal to 75° C., greater than or equal to 80° C., greater than or equal to 85° C., greater than or equal to 90° C., or greater than or equal to 95° C. In some embodiments, the process temperature may be less than or equal to 95° C., less than or equal to 90° C., less than or equal to 85° C., less than or equal to 80° C., less than or equal to 75° C., less than or equal to 70° C., less than or equal to 65° C., or less than or equal to 60° C.

In some embodiments the process time can be greater than or equal to 15 seconds, greater than or equal to 30 seconds, greater than or equal to 45 seconds, greater than or equal to 60 seconds, greater than or equal to 2 minutes, greater than or equal to 3 minutes, greater than or equal to 4 minutes, or greater than or equal to 5 minutes. In some cases, the process time may be less than or equal to 5 minutes, less than or equal to 4 minutes, less than or equal to 3 minutes, less than or equal to 2 minutes, less than or equal to 60 seconds, less than or equal to 45 seconds, less than or equal to 30 seconds, or less than or equal to 15 seconds.

For example, in some embodiments, the bonding method comprises exposing the body portion to a solvent to soften the polymer and heating the body portion to further soften the material. In some embodiments, the body portion is heated to a temperature of less than or equal to 100° C., less than 90° C., less than or equal to 80° C., less than or equal to 70° C., less than or equal to 60° C., or less than or equal to 50° C. In a set of preferred embodiments, the method comprises heating the body portion to a temperature of less than or equal to 100° C.

The body portion may be dehydrated, partially hydrated, or completed hydrated during the bonding process. In some embodiments, the water content of the body portion during bonding is greater than or equal to 5% w/w, greater than or equal to 10% w/w, greater than or equal to 20% w/w, greater than or equal to 30% w/w, greater than or equal to 40% w/w, or greater than or equal to 50% w/w. In certain embodiments, the water content of the body portion during bonding is less than or equal to 50% w/w, less than or equal to 40% w/w, less than or equal to 30% w/w, less than or equal to 20% w/w, less than or equal to 10% w/w, or less than or equal to 5% w/w.

In some embodiments, the body portion may be hydrated using any solvent known to those in the art. Preferred embodiments include the use of aqueous solvents, such as aqueous buffers. Exemplary embodiments include, but are not limited to, phosphate buffered saline, MEPS, TRIS, citric acid-sodium citrate buffers, citric acid-sodium phosphate buffers, sodium acetate-acetic acid buffers, imidazole buffer, and carbonate buffers. In some cases, the body portion and/or component may be hydrated for a set period of time (e.g., overnight) achieve equilibrium water content (e.g., steady state conditions—no more water is absorbed or released). Without wishing to be bound by any particular theory, it is generally known in the art that varying the concentration of the buffer solution can be used to control the equilibrium water content of the material, with higher salt concentrations resulting in higher hydration rates.

The period of hydration may, in some cases, be greater than or equal to 1 hour, greater than or equal to 5 hours, greater than or equal to 10 hours, greater than or equal to 15 hours, greater than or equal to 20 hours, or greater than or equal to 24 hours; in other cases, the period of hydration is less than or equal to 24 hours, less than or equal to 20 hours, less than or equal to 15 hours, less than or equal to 10 hours, less than or equal to 5 hours, or less than or equal to 1 hour.

The skilled artisan will understand that bonding may result in unwanted flowing/flashing of the materials being bonded together. Thus, in some cases, tooling designed to prevent the unwanted flowing/flashing of material may be used. Examples of tooling include, but are not limited to, split die bonder/RF welder with specific geometries and/or fluorinated ethylene propylene (FEP) (and/or PEEK, and/or Nylon, and/or polyolefin) heat shrink formed to the appropriate size and shape for hot air processing. The latter is particularly useful as compression of the materials promotes a strong contact patch and chain entanglement of the materials to be bonded.

In some embodiments, the method comprises cooling the integrated article. The article may be cooled using any technique known to those of skill in the art. In certain embodiments, the method comprises cooling the article at ambient temperature without any external control. However, in some cases, the method comprises cooling the article in a controlled manner, for example, by using an indirect heat exchanger. Additionally, or alternatively, the integrated article may be placed into an oven where cooling is achieved by systematically lowering the temperature in discrete intervals.

Some aspects of the current disclosure generally relate to methods of shaping an article (e.g., a catheter) comprising a body portion. In some embodiments, the body portion comprising a hydrophilic porous material comprising a lumen (e.g., a tubular structure). Shaping the article, in some embodiments, is achieved by first molding the hydrophilic porous material to a desired shape and then heating the hydrophilic porous material to lock in the desired structure. In certain embodiments, the hydrophilic porous material is heated prior to molding into the desired structure. Without wishing to be bound by any particular theory, it is generally believed that heating the hydrophilic porous material (e.g., poly(vinyl alcohol)) increases the chain mobility of the polymer chains within the hydrophilic porous material, thus allowing the material to be pliable or moldable at certain elevated temperatures, which upon cooling solidify and retain the desired shape.

In some embodiments, the hydrophilic porous material is heated to above its glass transition temperature but below its melting temperature. For example, in some embodiments, the hydrophilic porous material is heated to a temperature greater than or equal to 70° C., greater than or equal to 90° C., greater than or equal to 100° C., greater than or equal to 125° C., greater than or equal to 150° C., greater than or equal to 175° C., greater than or equal to 200° C., or greater than or equal to 225° C. In certain embodiments, the hydrophilic porous material is heated to a temperature of less than or equal to 225° C., less than or equal to 200° C., less than or equal to 175° C., less than or equal to 150° C., less than or equal to 125° C., less than or equal to 100° C., less than or equal to 90° C., or less than or equal to 70° C.

In some embodiments, shaping the hydrophilic porous material comprises bending it into a desired shape (e.g., twist, curve, circle, etc.,). In certain cases, bending the hydrophilic porous material comprises pressing the hydrophilic porous material into a mold having the desired shape. Additionally, or alternatively, bending the hydrophilic porous material comprises inserting a material into the lumen of the hydrophilic porous material ad bending the material to the desired shape (e.g., “V” shaped geometry”). Bending the hydrophilic porous material may also be accomplished by physically deforming the hydrophilic porous material (e.g., bending a distal end to a desired angle relative to a proximal end).

In some embodiments, the article may be shaped using thermal forming, for example, by placing a wire (nitinol, steel, etc.) inside a lumen and thermally heating the article to mold the it into the desired shape. Additionally, or alternatively, the article can be placed inside a metal housing (e.g., tube, mold cavity, etc.,) and thermally heated to mold the article into the desired shape. Shapes can be curved, straightened, compressed, or stretched by restricting the dimensions of the initial component to the desired shape. Edges can be rounded to allow ease of fluid flow, reduction of abrasion, and reduction of surface area, which can aid in catheter placement, catheter longevity, and patient comfort.

In some embodiments, the method of shaping comprises forming a dual lumen article (e.g., a dual lumen catheter) from a first body portion comprising a first lumen and a second body portion comprising a second lumen. Forming the dual lumen article may be accomplished, in some embodiments, by swelling the first body portion, thus rendering it pliable and/or bendable and placing the second body portion within the first lumen of the first body portion.

Exemplary solvents configured to swell the first body portion include, but are not limited to, water, dimethyl sulfoxide, and any combination thereof. For example, in some embodiments, the solvent comprises water in greater than or equal to 0% w/w, greater than or equal to 10% w/w, greater than or equal to 20% w/w, greater than or equal to 30% w/w, greater than or equal to 40% w/w, greater than or equal to 50% w/w, greater than or equal to 60% w/w, greater than or equal to 70% w/w, greater than or equal to 80% w/w, greater than or equal to 90% w/w, or greater than or equal to 100% w/w. In certain embodiments, the solvent comprises water in less than or equal to 100% w/w, less than or equal to 90% w/w, less than or equal to 80% w/w, less than or equal to 70% w/w, less than or equal to 60% w/w, less than or equal to 50% w/w, less than or equal to 40% w/w, less than or equal to 30% w/w, less than or equal to 20% w/w, less than or equal to 10% w/w, or less than or equal to 0% w/w. Combinations of the above-referenced ranges are also possible (e.g., greater than or equal to 10% w/w and less than or equal to 90% w/w).

In some embodiments, the solvent comprises DMSO in greater than or equal to 0% w/w, greater than or equal to 10% w/w, greater than or equal to 20% w/w, greater than or equal to 30% w/w, greater than or equal to 40% w/w, greater than or equal to 50% w/w, greater than or equal to 60% w/w, greater than or equal to 70% w/w, greater than or equal to 80% w/w, greater than or equal to 90% w/w, or greater than or equal to 100% w/w. In certain embodiments, the solvent comprises DMSO in less than or equal to 100% w/w, less than or equal to 90% w/w, less than or equal to 80% w/w, less than or equal to 70% w/w, less than or equal to 60% w/w, less than or equal to 50% w/w, less than or equal to 40% w/w, less than or equal to 30% w/w, less than or equal to 20% w/w, less than or equal to 10% w/w, or less than or equal to 0% w/w. Combinations of the above-referenced ranges are also possible (e.g., greater than or equal to 10% w/w and less than or equal to 90% w/w).

In a preferred set of embodiments, the solvent is a homogenous solution of DMSO. In another preferred set of embodiments, the solvent is a homogenous solution of water.

In some embodiments, the solvent causes the first body portion to swell (e.g., absorb the solvent within the non-crosslinked water-soluble polymer network of the body portion. Those of skill in the art will understand that the swelling ratio (e.g., the fractional increase in the weight of the material due to solvent absorption) may be used to quantify the degree of swelling of, for example, a hydrogel. A first body portion placed in a solvent may have a swelling ratio of greater than or equal to 10%, greater than or equal to 25%, greater than or equal to 50%, greater than or equal to 100%, greater than or equal to 250%, greater than or equal to 500%, greater than or equal to 1000%, greater than or equal to 2000%, or greater than or equal to 3000%, according to some embodiments. In other cases, the body portion placed in a solvent may have a swelling ratio of less than or equal to 3000%, less than or equal to 2000%, less than or equal to 1000%, less than or equal to 500%, less than or equal to 250%, less than or equal to 100%, less than or equal to 50%, less than or equal to 25%, or less than or equal to 10%.

In some embodiments, the second body portion is heated and/or mechanically deformed until an outer diameter of the second body portion is less than the first inner diameter of the first lumen (e.g., in the swollen state), thus permitting the second body portion to be placed inside the first body portion. As described elsewhere herein, it is generally believed that heating increases the mobility of the polymer chains that comprise the second body portion, thus allowing the second body portion to be manipulated (e.g., stretched and/or compressed, etc.). Any technique known to the skilled artisan may be used to reduce the outer diameter of the second body portion. For example, in some embodiments, the second body portion may be heated and stretched; additionally, or alternatively, a smaller diameter material may be placed into the lumen of the hydrophilic porous material and the second body radially compressed to the desired outer diameter.

In some embodiments, the outer diameter of the second body portion may vary along an axial dimension (e.g., the central axis). For example, in certain embodiments, the second body comprises a proximal end of a first outer dimension and a distal end of a second outer dimension, different than the first outer dimension. Configurations in which the second outer dimension is smaller than the first outer dimension, taper toward the distal end of the second body; on the contrary, configurations in which the first outer dimension is smaller than the second outer dimension, taper toward the proximal end of the second body. Other configurations are also possible. For example, the outer diameter of the second body portion may increase and decrease in a periodic fashion (e.g., sinusoidal) to produce a corrugated geometry. Other geometries and configurations are also possible.

Assembling the dual lumen article, in some embodiments, encompasses inserting the second body portion into the lumen of the first body portion and drying the dual lumen article. Without wishing to be bound by any particular theory, it is generally believed that drying the article removes the absorbed solvent from the first body portion causing the first body portion to shrink, thus entrapping the second body portion within the first body portion. In some embodiments, the dual lumen article is dried until the equilibrium water content is less than or equal to 5% w/w, less than or equal to 4% w/w, less than or equal to 3% w/w, less than or equal to 2% w/w, less than or equal to 1% w/w, less than or equal to 0.5% w/w, less than or equal to 0.25% w/w, less than or equal to 0.1% w/w, or less than or equal to 0.05% w/w.

In some embodiments, the dual lumen article comprises one or more surface features (e.g., barbs, bulges, hooks, etc.) at an interface between the first body portion and the second body portion configured to mechanically reinforce the interface between the first body portion and the second body portion. In certain embodiments, the first body portion comprises the one or more surface features; whereas in other cases, the second body portion may comprise the one or more surface features. In some cases, the first body portion comprises one or more surface features configured to engage the one or more surface features on the second body portion (e.g., similar to a lock and key). In some embodiments, drying the article causes the one or more surface features to engage the opposing surface, thus mechanically reinforcing the interface between the first body portion and the second body portion.

Some aspects of the present disclosure generally relate to methods of inserting any one of the articles described herein to a subject in need thereof. The article described herein are at least partially composed of a body portion comprising a first material comprising a hydrophilic water-soluble polymer. In some embodiments, the article is an intravenous catheter. Without wishing to be bound by any particular theory, it is believed that the processing parameters increase chain entanglement between the hydrophilic water-soluble polymers and non-covalent bonding between the adjacent polymer chains (e.g., hydrogen bonding), resulting in a physically crosslinked polymer network. Physically crosslinked hydrogels, like chemically crosslinked hydrogels, can absorb significant volumes of water within their crosslinked network. Thus, in some embodiments, the article may be inserted in a subject (e.g., intravenously) in a dehydrated state, a partially (de)hydrated state, or a completely hydrated state (e.g., swollen state).

In some embodiments, the article is in the dehydrated state when the article has a water content of less than or equal 5% w/w. The article is (at least partially) hydrated, according to some embodiments, when the article has a water content of greater than or equal to 5% w/w and less than or equal to 50% w/w. For example, in some embodiments, the article is in the (at least partially) hydrated state when the water content is greater than or equal to 5% w/w, greater than or equal to 10% w/w, greater than or equal to 20% w/w, greater than or equal to 30% w/w, greater than or equal to 40% w/w, or greater than or equal to 50% w/w. In certain embodiments, the article is in the hydrated state when the water content is less than or equal to 50% w/w, less than or equal to 40% w/w, less than or equal to 30% w/w, less than or equal to 20% w/w, less than or equal to 10% w/w, or less than or equal to 5% w/w.

In some embodiments, the article (e.g., intravenous catheter) comprises a polymer material comprising a first water-soluble polymer having a plurality of pores, a lumen, and a distal end comprising a tip geometry suitable for intravenous insertion into a subject. Non-limiting embodiments of possible tip geometries include a bevel tip needle stylet, bevel tip needle cannula, lancet point needle stylet, back bevel needle stylet, back bevel needle cannula, trocar tip needle stylet, Franseen tip needle cannula, or conical tip needle stylet. In some embodiments, the tip comprises a symmetric geometry; however, in certain embodiments the tip comprises an asymmetrical geometry.

In some embodiments, at least a portion of the article is in the dehydrated state and has a Young's elastic modulus of greater than or equal to 500 MPa, greater than or equal to 750 MPa, greater than or equal to 1 GPa, greater than or equal to 2 GPa, greater than or equal to 4 GPa, or greater than or equal to 5 GPa. In some embodiments, at least a portion of the article is in the dehydrated state and has a Young's elastic modulus of less than or equal to 10 GPa, less than or equal to 5 GPa, less than or equal to 4 GPa, less than or equal to 2 GPa, less than or equal to 1 GPa, or less than or equal to 750 MPa. Combinations of the above-referenced ranges are also possible (e.g., greater than or equal to 500 MPa and less than or equal to 10 GPa). Other ranges are also possible. In some embodiments, the article having at least a portion having a Young's elastic modulus of greater than or equal to 500 MPa is inserted into a subject (e.g., intravenously) without the aid of a needle or other venous puncturing device. For example, a portion of the article (e.g., a tip of the article) may have a needle-like design and sufficient Young's elastic modulus such that the article can penetrate the skin, subcutaneous tissue, and/or vein walls of the subject (e.g., facilitating needle-less insertion of a vascular catheter).

In some embodiments, at least a portion of the article may undergo rapid hydration (e.g., via flow of a fluid within the article (e.g., within a lumen of the article), via exposure to a biological fluid of the subject such as blood) upon insertion of the article into the subject. Advantageously, such articles may permit needleless insertion of a catheter intravenously into a subject such that the article remains within the patient (e.g., and serves as a catheter).

In some embodiments, the intravenous catheter is inserted in a dehydrated state. In some embodiments, the intravenous catheter has a first water content during insertion and a second water content after insertion. For example, the catheter may have a water content of less than or equal to 5% w/w during insertion and a water content greater than or equal to 5% w/w and less than or equal to 50% w/w following insertion. Without wishing to be bound by any particular theory, it is generally believed that upon insertion into the vein of a subject, the catheter will absorb blood components (e.g., serum, plasma, etc.,) within its physically crosslinked network and subsequently swell within the vein (e.g., the water content will increase) to a final dimension.

In some embodiments, the intravenous catheter may swell to its final dimension (e.g., at EWC) in greater than or equal to 5 seconds or less than or equal to 60 minutes. For example, in some embodiments, the intravenous catheter may swell to its final dimension in greater than or equal to 5 seconds, greater than or equal to 10 seconds, greater than or equal to 30 seconds, greater than or equal to 1 minute, greater than or equal to 5 minutes, greater than or equal to 10 minutes, greater than or equal to 20 minutes, greater than or equal to 30 minutes, greater than or equal to 40 minutes, greater than or equal to 50 minutes, or greater than or equal to 60 minutes. In certain embodiments, the intravenous catheter may swell to its final dimension is less than or equal to 60 minutes, less than or equal to 50 minutes, less than or equal to 40 minutes, less than or equal to 30 minutes, less than or equal to 20 minutes, less than or equal to 10 minutes, less than or equal to 5 minutes, less than or equal to 1 minute, less than or equal to 30 seconds, less than or equal to 10 seconds, less than or equal to 8 seconds. Combinations of the above-referenced ranges are also possible (e.g., greater than or equal to 5 seconds and less than or equal to 60 minutes, greater than or equal to 1 minute and less than or equal to 5 minutes, greater than or equal to 5 minutes and less than or equal to 30 minutes). Other ranges are also possible. Advantageously, the mechanical properties of the article may be tuned to have a particular swelling time (e.g., the time to swell to its final dimension at EWC), e.g., using the methods described herein.

In some embodiments, the intravenous catheter is inserted in a hydrated or partially hydrated state. Thus, in some cases, the catheter may have a water content of greater than or equal to 5% w/w and less than or equal to 50% w/w prior to insertion. In the case, where the catheter is not fully hydrated (e.g., less than 50%) prior to insertion, the catheter will continue to absorb fluid from the venous system until the equilibrium swelling ratio is achieved (e.g., 50%). In some embodiments, the catheter may have a water content of greater than or equal to 5% w/w, greater than or equal to 10% w/w, greater than or equal to 20% w/w, greater than or equal to 30% w/w, greater than or equal to 40% w/w, or greater than or equal to 50% w/w prior to insertion into a subject. In certain embodiments, the catheter may have a water content of less than or equal to 50% w/w, less than or equal to 40% w/w, less than or equal to 30% w/w, less than or equal to 20% w/w, less than or equal to 10% w/w, or less than or equal to 5% w/w prior to insertion into a subject. Combinations of the above-referenced ranges are also possible (e.g., greater than or equal to 5% w/w and less than or equal to 50% w/w).

In some embodiments, the inner diameter of the lumen is reduced following insertion into the patient. The person of skill in the art will understand, based upon the teachings of this specification, that the articles described herein, e.g., intravenous catheters, may be useful, for example, for administering (e.g., drugs, crystalloids, colloids, etc.,) and/or withdrawing (e.g., blood) fluids from a subject in need thereof. Importantly, inserting of the devices/articles of the present invention within the venous system of a subject does not cause substantial hemolysis and is thus considered generally safe.

Extrusion processes for making the articles of the present invention are described in International Patent Application Publication Nos. WO2018/237166 and WO2017/112878, which are hereby incorporated by reference in their entirety. In some embodiments, processes comprising extrusion are used so that devices with a high aspect ratio may be created. An embodiment of a process for making the materials involves heating a mixture that comprises at least one water-soluble polymer and a solvent to a temperature above the melting point of the polymer solution forming the mixture in a solvent-removing environment resulting in a physically crosslinked matrix and continuing to remove the solvent until the physically crosslinked matrix is a microporous or a nanoporous solid material. The physical crosslinking can take place while cooling the mixture and/or in the solvent-removing environment. Further polymers may be incorporated into pores of the material.

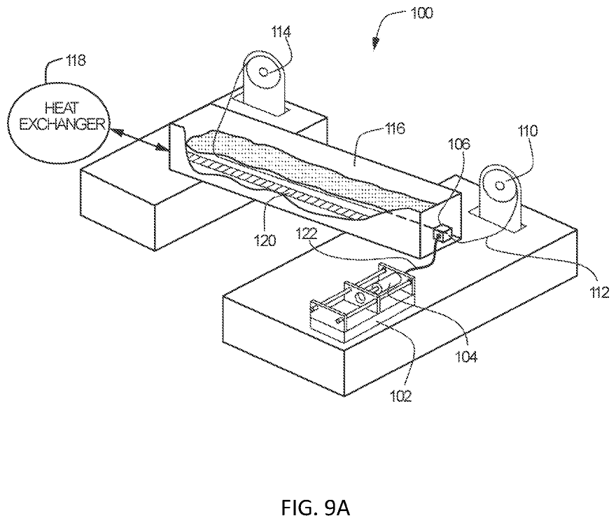

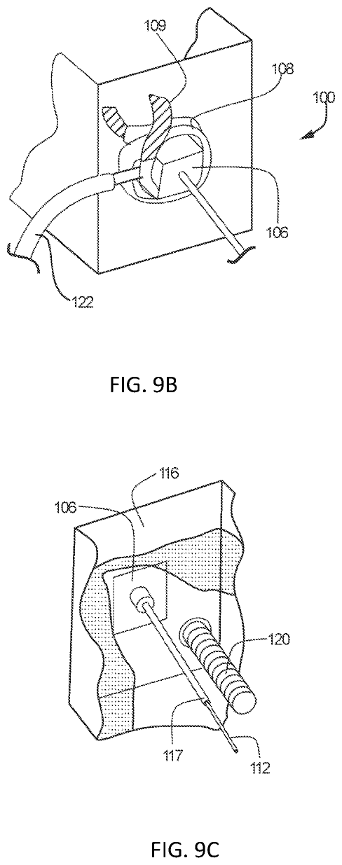

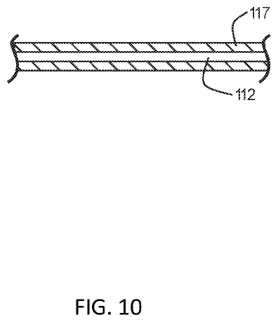

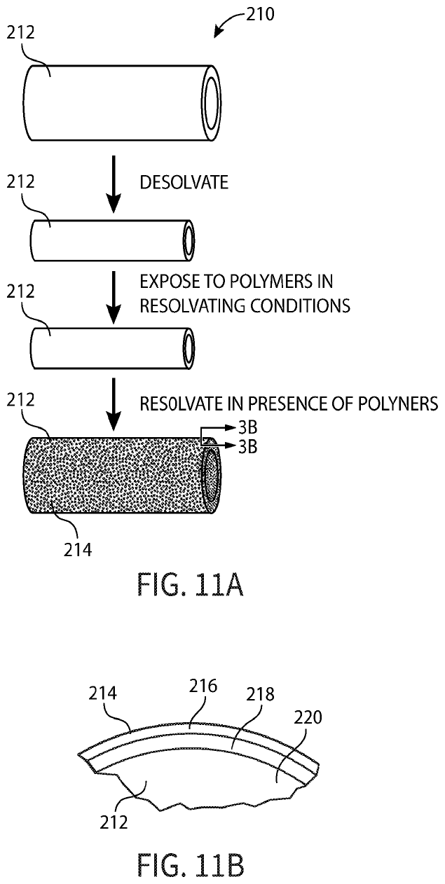

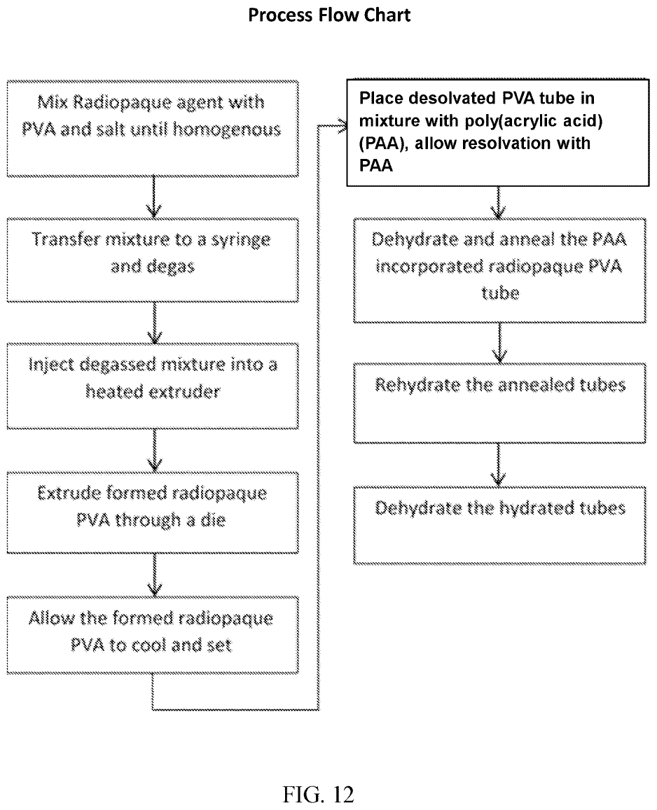

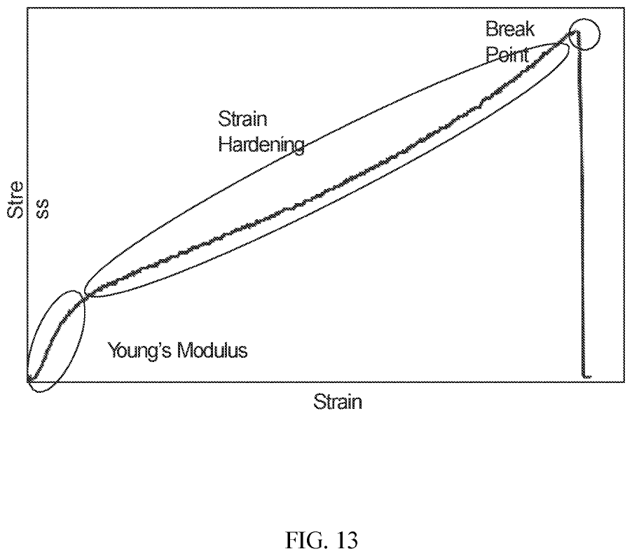

FIGS. 9A-9C depict an embodiment of an apparatus to make the porous solid materials. A device 100 as depicted includes a syringe pump 102 to accept at least one syringe 104, an optional heating jacket (not shown) to heat the syringes, die head 106, heating element 108 and power cables 109 for the same, providing heating as needed for die head 106 (detail not shown in FIG. 9A), dispensing spool 110 for core tubing 112, uptake spool 114 and motor (not shown) for core tubing, bath 116 for the extruded material 117, with the bath having temperature control for cooling or heating, depicted as heat exchanger 118 that comprises heat exchanging pipe 120 in bath 116. Die head 106 accepts the core tubing 110 which passes therethrough. Feed line 122 from the syringes to die head 106 provides a feed to device 100. A system for this embodiment may further include a weigh station, a jacketed vessel for heating and mixing solutions for loading into the syringes, and a solvent-removal environment for further drying of tubing removed from bath 116. The system may also have a heating station for annealing the tubing or other extrusion product with heat when desired. Core tubing made of PTFE as well as wires, air, gas, non-solvent liquid or other materials may be used for a core.

In use, by way of example, a polymer is heated in a suitable solvent in a jacketed vessel and placed into syringe 104. One or more polymers may be present and a radiopaque agent or other additive may be added. One or more syringes may be used with the same or different mixtures. The syringe(s) of the polymer are heated to a predetermined temperature, e.g., of no more than 80-95° C., and degassed before extrusion. Syringe 104 is mounted on syringe pump 102 with a wrap heater to maintain temperature during extrusion. Core 112 is looped through die head 106, e.g., a heated out-dwelling die head, which feeds into extrusion bath 116, and then attached to an uptake spool 114 that is driven by a motor. The temperature of the bath is controlled using heat exchanger 118, such as a chiller; extruded materials may be extruded at temperatures ranging from −30° C. to 75° C.; other temperatures may be used, and 0° C. is a generally useful temperature setting for extrusion. Artisans will immediately appreciate that all ranges and values between the explicitly stated bounds are contemplated, with, e.g., any of the following being available as an upper or lower limit: −30, −25, −20, −15, −10, −5, 0, 5, 10, 15, 20, 25, 30, 35, 40, 45, 50, 55, 60, 65, 70, 75° C. Uptake (e.g., puller) spool 114 motor speed can be controlled to adjust outer diameter gauge size around core 112. Adjusting die size, material feed rate, tubing core diameter, and puller speed play roles in adjusting final tubing gauge, e.g., in embodiments wherein a catheter is made. Polymer feed rates are adjustable, e.g., by control of syringe pump 102 in this embodiment. Connectors 122 join the one or more syringes to die head 106. Many pumps and other tools for controllably feeding a polymer solution are known. The apparatus and method can be adapted for a drawing process although alternative feed processes are available.

In some embodiments, a composition (e.g., a pre-polymer composition) may be provided (e.g., for extrusion) prior to formation of the polymeric material. In some embodiments, the composition comprises an aqueous solution. The aqueous solution can comprise an osmotic agent at a concentration of greater than or equal to 0.01 M and less than or equal to 8 M. The aqueous solution can comprise a radiopaque agent in an amount of greater than or equal to 0 w/w % and less than or equal to 50 w/w % (e.g., less than or equal to 40 w/w %). The composition can further comprise a water-soluble polymer having a molecular weight of greater than or equal to 40 kDa and less than or equal to 5000 kDa, and present in the solution in an amount greater than or equal to 10 w/w % and less than or equal to 50 w/w %.

In some embodiments, the composition forms a swellable polymeric material upon extrusion.

In some embodiments, the osmotic agent is present in the solution at a concentration of greater than or equal to 0.01 M, greater than or equal to 0.1 M, greater than or equal to 0.5 M, greater than or equal to 1 M, greater than or equal to 2 M, greater than or equal to 3 M, greater than or equal to 4 M, greater than or equal to 5 M, or greater than or equal to 6 M. In some embodiments, the osmotic agent is present in the solution at a concentration of less than or equal to 8 M, less than or equal to 6 M, less than or equal to 4 M, less than or equal to 2 M, less than or equal to 1 M, less than or equal to 0.5 M, or less than or equal to 0.1 M. Combinations of the above-referenced ranges are also possible (e.g., greater than or equal to 0.01 M and less than or equal to 8 M). Osmotic agents are described in more detail herein.

In some embodiments, the radiopaque agent is present in the solution in an amount of greater than or equal to 0 w/w %, greater than or equal to 5 w/w %, greater than or equal to 10 w/w %, greater than or equal to 15 w/w %, greater than or equal to 20 w/w %, greater than or equal to 25 w/w %, greater than or equal to 30 w/w %, greater than or equal to 35 w/w %, greater than or equal to 40 w/w %, or greater than or equal to 45 w/w %. In some embodiments, the radiopaque agent is present in the solution in an amount less than or equal to 50 w/w %, less than or equal to 45 w/w %, less than or equal to 40 w/w %, less than or equal to 35 w/w %, less than or equal to 30 w/w %, less than or equal to 25 w/w %, less than or equal to 20 w/w %, less than or equal to 15 w/w %, less than or equal to 10 w/w %, or less than or equal to 5 w/w %. Combinations of the above-referenced ranges are also possible (e.g., greater than or equal to 0 w/w % and less than or equal to 50 w/w %). Other ranges are also possible. Radiopaque agents are described in more detail, below.

In some embodiments, the water-soluble polymer is present in the solution in an amount greater than or equal to 10 w/w %, greater than or equal to 13 w/w %, greater than or equal to 15 w/w %, greater than or equal to 20 w/w %, greater than or equal to 25 w/w %, greater than or equal to 30 w/w %, greater than or equal to 35 w/w %, greater than or equal to 40 w/w %, or greater than or equal to 45 w/w %. In some embodiments, the water-soluble polymer is present in the solution in an amount less than or equal to 50 w/w %, less than or equal to 45 w/w %, less than or equal to 40 w/w %, less than or equal to 35 w/w %, less than or equal to 30 w/w %, less than or equal to 25 w/w %, less than or equal to 20 w/w %, less than or equal to 15 w/w %, or less than or equal to 13 w/w %. Combinations of the above-referenced ranges are also possible (e.g., greater than or equal to 10 w/w % and less than or equal to 50 w/w %). In some embodiments, the water-soluble polymer is present in the solution in an amount greater than or equal to 13 w/w %.

In some embodiments, the method for forming the polymeric materials and/or devices described herein comprises providing a mixture comprising a first water-soluble polymer and an osmotic agent (e.g., a salt) as described above. In some embodiments, the mixture is extruded. In some embodiments, the extruded mixture is extruded on a core material to form the polymeric material disposed on the core material. In some embodiments, the formed polymeric material is exposed to a non-solvent of the polymeric material. In some embodiments, a solution comprising a second water-soluble polymer different that the first water-soluble polymer and, optionally, an osmotic agent, is introduced to the polymeric material. In some embodiments, the polymeric material (e.g., after introducing the solution to the osmotic agent) is heated. In some embodiments, the solution is flowed against the polymeric material. In some embodiments, the polymeric material may be dried.

In an exemplary set of embodiments, the method for forming the polymeric materials and/or devices described herein comprises physically integrating a body portion with one or more components. In some embodiment, the method comprises providing a mixture comprising a first water-soluble polymer and an osmotic agent (e.g., a salt), wherein the first water-soluble polymer is present in the mixture in an amount greater than or equal to 10 w/w % (e.g., greater than or equal to 13 w/w % or greater than or equal to 13 w/w % and less than or equal to 50 w/w %) versus the total weight of the mixture, performing the steps of: extruding the mixture at a temperature greater than or equal to 65° C. (e.g., greater than or equal to 65° C. and less than or equal to 100° C.) at atmospheric pressure, on a core material to form the polymeric material disposed on the core material (e.g., a solid rod or a gas), exposing the polymeric material to a non-solvent of the polymeric material at a temperature less than or equal to 28° C. (e.g., less than or equal to 28° C. and greater than or equal to −20° C.) for greater than or equal to 15 minutes (e.g., greater than or equal to 1 hour and less than or equal to 240 hours), placing the polymeric material in contact with a component comprising a second material, different than the first water-soluble polymer, and/or an osmotic agent (e.g., a salt) to form an integrated article, heating the integrated article to a temperature of greater than or equal to 25° C. (e.g., greater than or equal to 30° C., or greater than or equal to 30° C. and less than or equal to 65° C.), for example, for greater than or equal to 1 hour (e.g., greater than or equal to 1 hour and less than or equal to 48 hours or greater than or equal to 3 hours and less than or equal to 48 hours) to dry the polymeric material.

In some embodiments, the non-solvent comprises alcohol. In some embodiments, the non-solvent is ethanol, methanol, propanol, butanol, pentanol, hexanol, heptanol, octanol, decanol, dodecanol, dimethyl sulfoxide, ethyl acetate, acetates, propionates, ethers, dimethyl formamide, dimethyl acetamide, acetone, acetonitrile, ethylene glycol, propylene glycol, glycerol air, vacuum or combinations thereof. Other non-solvents are also possible (e.g., a solvent having a high solubility to water but a lower solubility to the water-soluble polymer, as compared to the solubility in water).

In some embodiments, the step of extruding the mixture is performed under atmospheric pressure at a temperature of greater than or equal to 65° C., greater than or equal to 70° C., greater than or equal to 75° C., greater than or equal to 80° C., greater than or equal to 85° C., greater than or equal to 90° C., greater than or equal to 95° C., greater than or equal to 100° C., or greater than or equal to 105° C. In some embodiments, the step of extruding the mixture is performed under atmospheric pressure at a temperature of less than or equal to 110° C., less than or equal to 105° C., less than or equal to 100° C., less than or equal to 95° C., less than or equal to 90° C., less than or equal to 85° C., less than or equal to 80° C., less than or equal to 75° C., or less than or equal to 70° C. Combinations of the above-referenced ranges are also possible (e.g., greater than or equal 5 to 65° C. and less than or equal to 110° C.). Other ranges are also possible. Those of ordinary skill in the art would understand, based upon the teachings of this specification, that additional pressures (e.g., greater than atmospheric pressure, less than atmospheric pressure) and/or temperatures are also possible.

In some embodiments, the step of exposing the polymeric material to a non-solvent of the polymeric material is performed at a temperature less than or equal to 28° C., less than or equal to 25° C., less than or equal to 20° C., less than or equal to 15° C., less than or equal to 10° C., less than or equal to 5° C., less than or equal to 0° C., less than or equal to −5° C., less than or equal to −10° C., or less than or equal to −15° C. In some embodiments, the step of exposing the polymeric material to a non-solvent of the polymeric material is performed at a temperature greater than or equal to −20° C., greater than or equal to −15° C., greater than or equal to −10° C., greater than or equal to −5° C., greater than or equal to 0° C., greater than or equal to 5° C., greater than or equal to 10° C., greater than or equal to 15° C., greater than or equal to 20° C., or greater than or equal to 25° C. Combinations of the above-referenced ranges are also possible (e.g., less than or equal to 28° C. and greater than or equal to −20° C.). Other ranges are also possible.

In some embodiments, the step of exposing the polymeric material to the non-solvent of the polymeric material is performed (e.g., at a temperature less than or equal to 28° C. and greater than or equal to −20° C.) for greater than or equal to 1 hour, greater than or equal to 2 hours, greater than or equal to 4 hours, greater than or equal to 6 hours, greater than or equal to 8 hours, greater than or equal to 10 hours, greater than or equal to 15 hours, greater than or equal to 20 hours, greater than or equal to 30 hours, greater than or equal to 40 hours, greater than or equal to 50 hours, greater than or equal to 60 hours, greater than or equal to 80 hours, greater than or equal to 100 hours, greater than or equal to 120 hours, greater than or equal to 140 hours, greater than or equal to 160 hours, greater than or equal to 180 hours, greater than or equal to 200 hours, or greater than or equal to 220 hours. In some embodiments, the step of exposing the polymeric material to the non-solvent of the polymeric material is performed for less than or equal to 240 hours, less than or equal to 220 hours, less than or equal to 200 hours, less than or equal to 180 hours, less than or equal to 160 hours, less than or equal to 140 hours, less than or equal to 120 hours, less than or equal to 100 hours, less than or equal to 80 hours, less than or equal to 60 hours, less than or equal to 50 hours, less than or equal to 40 hours, less than or equal to 30 hours, less than or equal to 20 hours, less than or equal to 15 hours, less than or equal to 10 hours, less than or equal to 8 hours, less than or equal to 6 hours, less than or equal to 4 hours, or less than or equal to 2 hours. Combinations of the above-referenced ranges are also possible (e.g., greater than or equal to 1 hour and less than or equal to 240 hours). Other ranges are also possible.

In some embodiments, the step of introducing to the polymeric material, a component comprising a second material, different than the first water-soluble polymer, and an optional osmotic agent (e.g., a salt) comprises heating the polymeric material and the component to a temperature of greater than or equal to 25° C., greater than or equal to 30° C., greater than or equal to 35° C., greater than or equal to 40° C., greater than or equal to 45° C., greater than or equal to 50° C., greater than or equal to 55° C., or greater than or equal to 60° C. In some embodiments, the polymeric material and the component are heated to a temperature less than or equal to 65° C., less than or equal to 60° C., less than or equal to 55° C., less than or equal to 50° C., less than or equal to 45° C., less than or equal to 40° C., less than or equal to 35° C., or less than or equal to 30° C. Combinations of the above-referenced ranges are also possible (e.g., greater than or equal to 25° C. and less than or equal to 65° C.). Other ranges are also possible.

In some cases, it may be desirable to incorporate one or more biological molecules within the integrated article as described in International Patent Application Publication No WO2020/132065. Thus, in some embodiments, a solution comprising one or more compounds agents of interest (therapeutics, biological agents, etc.), and optionally, an osmotic agent, may be flowed adjacent (e.g., directly adjacent) to the integrated article for a particular amount of time. In some embodiments, the solution is flowed adjacent the integrated article for greater than or equal to 3 hours, greater than or equal to 5 hours, greater than or equal to 6 hours, greater than or equal to 8 hours, greater than or equal to 10 hours, greater than or equal to 12 hours, greater than or equal to 16 hours, greater than or equal to 20 hours, greater than or equal to 24 hours, greater than or equal to 28 hours, greater than or equal to 32 hours, greater than or equal to 36 hours, greater than or equal to 40 hours, or greater than or equal to 44 hours. In some embodiments, the solution is flowed adjacent the integrated article for less than or equal to 48 hours, less than or equal to 44 hours, less than or equal to 40 hours, less than or equal to 36 hours, less than or equal to 32 hours, less than or equal to 28 hours, less than or equal to 24 hours, less than or equal to 20 hours, less than or equal to 16 hours, less than or equal to 12 hours, less than or equal to 10 hours, less than or equal to 8 hours, less than or equal to 6 hours, or less than or equal to 5 hours. Combinations of the above-referenced ranges are also possible (e.g., greater than or equal to 3 hours and less than or equal to 48 hours). Other ranges are also possible. Combinations of the above-referenced temperatures and times are also possible.

In some embodiments, the method comprises annealing the integrated article to a temperature of greater than or equal to 80° C. (e.g., greater than or equal to 80° C. and less than or equal to 250° C.) for greater than or equal to 60 minutes (e.g., greater than or equal to 60 minutes and less than or equal to 480 minutes). In some embodiments, the integrated article is annealed at a temperature of greater than or equal to 80° C., greater than or equal to 90° C., greater than or equal to 100° C., greater than or equal to 120° C., greater than or equal to 140° C., greater than or equal to 160° C., greater than or equal to 180° C., greater than or equal to 200° C., greater than or equal to 220° C., or greater than or equal to 240° C. In some embodiments, the integrated article is annealed at a temperature of less than or equal to 250° C., less than or equal to 240° C., less than or equal to 220° C., less than or equal to 200° C., less than or equal to 180° C., less than or equal to 160° C., less than or equal to 140° C., less than or equal to 120° C., less than or equal to 100° C., or less than or equal to 90° C. Combinations of the above-referenced ranges are also possible (e.g., greater than or equal to 80° C. and less than or equal to 250° C.). Other ranges are also possible.

In some embodiments, the integrated article (e.g., body portion and component) is annealed for greater than or equal to 30 minutes, greater than or equal to 60 minutes, greater than or equal to 80 minutes, greater than or equal to 100 minutes, greater than or equal to 120 minutes, greater than or equal to 160 minutes, greater than or equal to 200 minutes, greater than or equal to 240 minutes, greater than or equal to 280 minutes, greater than or equal to 320 minutes, greater than or equal to 360 minutes, greater than or equal to 400 minutes, or greater than or equal to 440 minutes. In some embodiments, the integrated article is annealed for less than or equal to 480 minutes, less than or equal to 440 minutes, less than or equal to 400 minutes, less than or equal to 360 minutes, less than or equal to 320 minutes, less than or equal to 280 minutes, less than or equal to 240 minutes, less than or equal to 200 minutes, less than or equal to 160 minutes, less than or equal to 120 minutes, less than or equal to 100 minutes, or less than or equal to 80 minutes. Combinations of the above-referenced ranges are also possible (e.g., greater than or equal to 60 minutes and less than or equal to 480 minutes). Other ranges are also possible. Combinations of the above-referenced temperatures and times are also possible.

In some embodiments, the core material may be air, water, a non-solvent liquid, a solid, or a gas. In some cases, the core material may be removed after formation of the polymeric material (e.g., body portion) on the core material. The core material may be physically removed and/or dissolved, in some cases.

In an exemplary embodiment, the method comprises, with a mixture (e.g., a solution as described above and herein) comprising at least one water-soluble polymer, a salt, and water, wherein the at least one water-soluble polymer is present in the mixture in an amount greater than or equal to 10 w/w % versus the total weight of the mixture, performing the steps of: heating the mixture to a temperature greater than or equal to 65° C., after heating the mixture, cooling the mixture to a temperature at least 20° C. cooler than a melting point of the mixture and mechanically shaping the mixture. In some embodiments, after cooling the mixture, the mixture may be extruded at a temperature greater than or equal to 65° C. on a core material to form the polymeric material disposed on the core material. The method may involve exposing the polymeric material to non-solvent of the polymeric material at a temperature less than or equal to 28° C. for greater than or equal to 4 hours and removing at least a portion of the core material from the polymeric material.

In some embodiments, the step of cooling the mixture comprises cooling to a temperature at least 20° C., at least 25° C., at least 30° C., at least 35° C., at least 40° C., at least 45° C., at least 50° C., at least 60° C., at least 70° C., at least 80° C., or at least 90° C. cooler than a melting point of the mixture. In some embodiments, the step of cooling the mixture comprises cooling to a temperature of less than or equal to 100° C., less than or equal to 90° C., less than or equal to 80° C., less than or equal to 70° C., less than or equal to 60° C., less than or equal to 50° C., less than or equal to 45° C., less than or equal to 40° C., less than or equal to 35° C., less than or equal to 30° C., or less than or equal to 25° C. lower than a melting point of the mixture. Combinations of the above-referenced ranges are also possible (e.g., at least 20° C. and less than or equal to 100° C. lower). Other ranges are also possible. The mixture may be cooled for any suitable amount of time.

In some embodiments, the mixture may be mechanically shaped. In some embodiments, the composition (e.g., prior to extrusion i.e. the mixture) may be mechanically shaped by kneading, rolling, cutting, and combinations thereof.

In some embodiments, the mixture is mixed at a temperature of greater than or equal to 80° C., greater than or equal to 90° C., greater than or equal to 100° C., greater than or equal to 120° C., greater than or equal to 140° C., greater than or equal to 160° C., greater than or equal to 180° C., greater than or equal to 200° C., greater than or equal to 220° C., or greater than or equal to 240° C. In some embodiments, the mixture is mixed at a temperature of less than or equal to 250° C., less than or equal to 240° C., less than or equal to 220° C., less than or equal to 200° C., less than or equal to 180° C., less than or equal to 160° C., less than or equal to 140° C., less than or equal to 120° C., less than or equal to 100° C., or less than or equal to 90° C. Combinations of the above-referenced ranges are also possible (e.g., greater than or equal to 80° C. and less than or equal to 250° C.). Other ranges are also possible.

In some embodiments, the method comprises sorption of a compound of interest (e.g., drug) into the polymeric material, as described above and herein.

In some embodiments, the polymeric materials and/or devices described herein may be exposed to and/or comprise a humectant. In some embodiments, at least a portion of the humectant is disposed on a surface (e.g., an inner lumen and/or an abluminal surface) of the polymeric material and/or device (e.g., the body portion). For example, in some embodiments, a portion of humectant 70 is disposed on a surface of device 10. In some embodiments, greater than or equal to 30%, greater than or equal to 40%, greater than or equal to 50%, greater than or equal to 60%, greater than or equal to 70%, greater than or equal to 80%, greater than or equal to 90%, or all of the humectant is disposed on a surface of the polymeric material and/or device (e.g., the body portion). In some embodiments, less than or equal to 100%, less than or equal to 90%, less than or equal to 80%, less than or equal to 70%, less than or equal to 60%, less than or equal to 50%, less than or equal to 40% of the humectant is disposed on a surface of the polymeric material and/or device (e.g., the body portion). Combinations of these ranges are also possible (e.g., 40-100%).

In some embodiments, at least a portion of the humectant is inside the polymeric material and/or device (e.g., the body portion). In some embodiments, at least a portion of the humectant is inside the polymeric material and/or device (e.g., the body portion). For example, in some embodiments, a portion of humectant 70 is inside device 10 (e.g., absorbed into the bulk of the device). In some embodiments, greater than or equal to 30%, greater than or equal to 40%, greater than or equal to 50%, greater than or equal to 60%, greater than or equal to 70%, greater than or equal to 80%, greater than or equal to 90%, or all of the humectant is inside the polymeric material and/or device (e.g., the body portion). In some embodiments, less than or equal to 100%, less than or equal to 90%, less than or equal to 80%, less than or equal to 70%, less than or equal to 60%, less than or equal to 50%, less than or equal to 40% of the humectant is inside the polymeric material and/or device (e.g., the body portion). Combinations of these ranges are also possible (e.g., 30-100%).

In some embodiments, the humectant is a non-ionic surfactant (i.e. a surfactant having an uncharged hydrophilic head and a hydrophobic tail) or a zwitterionic surfactant (i.e. a surfactant having a net uncharged hydrophilic head and a hydrophobic tail). In some embodiments, the humectant is a non-ionic surfactant selected from the group consisting of sugar alcohols, poloxamer, triacetin, α-hydroxy acids, polyethylene glycol, polypropylene glycol, ethylene glycol, propylene glycol, hexylene glycol, butylene glycol, glycerol, sorbitol, mannitol, xylitol, maltitol, erythritol, threitol, arabitol, ribitol, galactitol, fucitol, iditol, inositol, volemitol, malitol, lactitol, maltotriitol, maltotetraitol, polyglycitols, and combinations thereof. In some embodiments, the humectant comprises an oil such as vitamin E. In some embodiments, the humectant comprises a salt such as sodium chloride, potassium chloride, and/or phosphocholine.

In some embodiments, the polymeric materials and/or devices described herein are exposed to and/or comprise greater than or equal to 0.1 w/w % humectant, greater than or equal to 0.5 w/w % humectant, greater than or equal to 1 w/w % humectant, greater than or equal to 5 w/w % humectant, greater than or equal to 10 w/w % humectant, or greater than or equal to 20 w/w % humectant. In some embodiments, the polymeric materials and/or devices described herein are exposed to and/or comprise less than or equal to 30 w/w % humectant, less than or equal to 25 w/w % humectant, less than or equal to 20 w/w % humectant, less than or equal to 15 w/w % humectant, less than or equal to 10 w/w % humectant, less than or equal to 5 w/w % humectant, or less than or equal to 1 w/w % humectant. Combinations of these ranges are also possible (e.g., 0.1-30 w/w % humectant or 1-10 w/w % humectant). A porous solid (e.g., made by the apparatus of FIGS. 1D-1F) may be annealed. Further, a porous solid, with or without prior annealing, may be processed to further include bulk incorporated polymers. In FIG. 3A, material 210 comprising porous solid matrix 212 is desolvated, exposed to a mixture comprising polymers that are in a resolvating solvent, and resolvated in the mixture to form material 212 with bulk incorporated polymers 214. A cross section of matrix 212 (FIG. 3B) reveals an outermost zone 216 wherein pores of matrix 212 are filled, an intermediate zone 218 wherein there is a lesser density of polymers in the pores, with less filling and/or fewer of the pores being occupied, and an inner zone 220 wherein polymers have not penetrated. The matrix can be solvated and/or desolvated prior to exposure to the mixture, provided that it is desolvated when exposed to the mixture so that water-soluble polymers can be moved into the matrix.

In some embodiments, a method for humectifying a device and/or polymeric material comprises placing an extruded segment into a solution comprising the humectant (e.g., glycerol or poloxamer). In some embodiments the solution comprises greater than or equal to 1 w/w %, greater than or equal to 5 w/w %, greater than or equal to 10 w/w %, greater than or equal to 15 w/w %, greater than or equal to 20 w/w %, or greater than or equal to 25 w/w % humectant. In some embodiments, the solution comprises less than or equal to 35 w/w %, less than or equal to 30 w/w %, less than or equal to 25 w/w %, less than or equal to 20 w/w %, less than or equal to 15 w/w %, less than or equal to 10 w/w %, or less than or equal to 5 w/w % humectant. Combinations of these ranges are also possible (e.g., 1-35 w/w %). In some embodiments, the solution comprises a surfactant. In some embodiments, the solution comprises PBS.

In some embodiments, the extruded segment is placed in the solution for a period of time. In some embodiments, the period of time is greater than or equal to 1 hour, greater than or equal to 2 hours, or greater than or equal to 3 hours. In some embodiments, the period of time is less than or equal to 4 hours, less than or equal to 3 hours, or less than or equal to 2 hours. Combinations of these ranges are also possible (e.g., 3 hours, or 1-4 hours).

In some embodiments, the solution is maintained at a temperature during exposure of the extruded segment to the solution. In some embodiments, the temperature is greater than or equal to 20° C., greater than or equal to 30° C., greater than or equal to 37° C., greater than or equal to 40° C., greater than or equal to 50° C., or greater than or equal to 60° C. In some embodiments, the temperature is less than or equal to 70° C., less than or equal to 60° C., less than or equal to 55° C., less than or equal to 50° C., less than or equal to 40° C., less than or equal to 37° C., or less than or equal to 30° C. Combinations of these ranges are also possible (e.g., 20-70° C., 37-55° C., or 45° C.).

In some embodiments, after the extruded segment is removed from the solution, the extruded segment can be dried (e.g., in a convection oven). In some embodiments, the extruded segment is dried at a certain temperature. In some embodiments, the temperature is greater than or equal to greater than or equal to 20° C., greater than or equal to 30° C., or greater than or equal to 40° C. In some embodiments, the temperature is less than or equal to 50° C., less than or equal to 40° C., or less than or equal to 30° C. Combinations of these ranges are also possible (e.g., 30° C., or 20-50° C.). In some embodiments, the extruded segment is dried for a period of time. In some embodiments, the period of time may be greater than or equal to 1 hour, greater than or equal to 2 hours, or greater than or equal to 3 hours. In some embodiments, the period of time may be less than or equal to 4 hours, less than or equal to 3 hours, or less than or equal to 2 hours. Combinations of these ranges are also possible (e.g., 3 hours, or 1-4 hours).

In some embodiments, a biologically active agent may be incorporated into the article.

In some embodiments, the methods described herein are free of freeze-thaw processes and/or free of a freezing process and/or free of a thawing process. Further the methods can be used to make solid porous materials that have little or no swelling, e.g., 0%-100% w/w swelling at EWC, even in an absence of covalent crosslinking agents. Artisans will immediately appreciate that all ranges and values between the explicitly stated bounds are contemplated, with, e.g., any of the following being available as an upper or lower limit: 0, 5, 10, 15, 20, 25, 30, 40, 50, 60, 70, 80, 90, 95, 100% w/w, with swelling measured as % swelling=100×(Total weight at EWC-dry weight)/dry weight, with the dry weight being the weight of the material without water.

In some embodiments, the extruded samples have a horizontal chain orientation and alignment along the length of samples (in direction of extrusion). A polymeric chain orientation produced by the extrusion process. Without wishing to be bound by theory, it is believed that this horizontal chain orientation and alignment along the length of the samples contributes to the inner diameter and/or outer diameter increasing by a larger percentage than the percentage increase in length when the samples swell, in some embodiments.

In some embodiments, it is useful to have a combination of one or more of: extrusion of a hydrophilic polymer in a solvent; a cold extrusion, and extrusion into a bath that quickly removes solvent from the extrudate. Further, in some embodiments, additional solvent-removing and/or annealing processes provide further utility for making desirable porous solids.

In some embodiments, requirements for a nanoporous material include high polymer concentrations of more than about 10% w/w in the polymer-solvent mixture with high levels of crosslinking. Artisans will immediately appreciate that all ranges and values between the explicitly stated bounds are contemplated, with, e.g., any of the following being available as an upper or lower limit: 10, 12, 15, 16, 17, 18, 19, 20, 25, 30, 35, 40, 45, 50, 55, 60, 70, 80, 90, 95, 99% w/w of the polymer in the total weight of the polymer-solvent mixture. In some embodiments, the polymer is to be substantially solvated, meaning it is a true solution or at least half the polymer is dissolved and the rest is at least suspended. In some embodiments, the solvation of the polymer contributes to the alignment of the polymer chains in an extrusion and to crosslinking among the polymers. Without being bound to a particular theory, it is likely that high concentration of the starting polymer-solvent mixture can help with this. And the probable chain alignment of the material as it passes through a die, according to some embodiments, is thought to promote more intrapolymer versus interpolymer crosslinking. An extrudate or an otherwise formed mixture entering a desolvating environment, whether gas or liquid, is thought to further collapse pore structure before the densely concentrated polymer has completely crosslinked, in some embodiments, thereby improving chain proximity and promoting additional crosslink density. Depositing the extruded or otherwise formed material directly into a solvent removing environment is helpful in some embodiments. In some embodiments, further solvent-removal can be continued to collapse the material until reaching a desired end point in structure and/or properties. An annealing process can further contribute to strength in some embodiments.

Frozen methods, on the other hand, rely on increased strengthening by forcing super-concentrated microregions to also achieve chain proximity and improve crosslink density, but retain a macro porosity due to the presence of ice crystals in the total gel structure. Desolvation creates forced super-concentrated microregions but these do not create macropores. In contrast, a pre-established gel prior to a dehydration or freezing is by nature of that process formed with macropores. Further, the work of the inventors indicates that such nanoporous solids have greater strength than macroporous materials.

Hydrogels can also be made by using a lower polymer concentration in the polymer-solvent mixture, generally less than 10% w/w of polymer in the polymer-solvent mixture. Artisans will immediately appreciate that all ranges and values between the explicitly stated bounds are contemplated, with, e.g., any of the following being available as an upper or lower limit: 2, 5, 7, 8, 9, 10% w/w of the polymer in the total weight of the polymer-solvent mixture. Further, or alternatively, the polymer-solvent mixture is not extruded into a solvent removing environment.

Microporous materials may be made with process conditions intermediate to nanoporous solids and hydrogels. One embodiment is to prepare a material using conditions comparable to making a nanoporous material but to stop solvent removal before solvent removal reaches a nanoporous solid structure.

Extrusion of hydrophilic polymers in a solvent is helpful to make high strength materials. Use of a solvent in an extrusion starting material is, at the least, uncommon. Typically, an extrusion uses a solid material that has been heated to a flowable temperature and then extruded, and later cooled by a variety of methods. For instance, it is believed that thermoplastic extrusion of a pure PVA is possible. But such an extrusion would lack the polymeric structure that is needed to make porous solids and would instead exhibit properties more similar to a conventional thermoplastic material. According to a theory of operation, a pure PVA extrusion would lack the quality of hydrogen bonding that takes place in an aqueous ionic solvent state. A temperature suitable for preparing the PVA to be flowable in an extrusion would create a poorly cohesive material at the die head so that a continuous shape does not form. It was difficult to make extruded PVAs to form high aspect shapes, e.g., tubes, and to use them in an extrusion process. Viscosities of PVA and other hydrophilic polymers are high, and difficult to get into solution. It was observed that a narrow working band of temperature was particularly useful, e.g., 85-95° C. Below about 85° C., PVA failed to truly melt, and thus did not become completely amorphous for extrusion. Above about 95° C., losses to boiling and evaporation made the process ineffective. These temperature ranges could be offset by increasing pressure above atmospheric, but a pressurized system is challenging to use and to scale. The processes are usefully performed at a temperature below a boiling point of the polymer-solvent materials.

The cohesive strength of the flowing polymer-solvent mixture was weak when exiting the die. The use of a core to support the mixture at the die is useful to hold the shape at the die. This condition is in contrast to a typical core extrusion used as a coating process, e.g., for coating wires for a mobile telephone charger. A typical process that avoids use of a solvent or a significant solvent concentration has a relatively higher cohesive strength that it exits the die that is readily capable of holding a tube, and do not relying on active bonding such as the hydrogen bonding in hydrophilic polymers that form the solid material in a coherent shape as it moves out of the die.

Passing the formed polymer-solvent mixture into solvent removal environment was useful. Most extrusions do not use bath temperatures at or below room temperature. Moreover, the use of a solvent removing bath is atypical relative to conventional processes the bath or other solvent removing environment helps solidify the extruded material sufficiently that it remains stable and concentric on the core, otherwise the melt would run into a tear drop shape. It would also be destroyed in the attempt to collect it at the end of the extrusion as it would still be molten. Conventional baths containing water would cause the PVA or similar hydrophilic polymer material to lose shape due to swelling, dissolution, or both. Molding processes that involve preparation of a polymer-solvent mixture that is formed in a mold and then processed into a solvent-removing environment do not have the advantages of alignment of chains observed in an extrusion. However, a suitably controlled temperature and solvent removal can yield materials with a high strength and controlled pore structure.

The porous solids are highly lubricious and can be used in a hydrated state and can be conveniently bonded to other materials. In the case of a catheter, for instance, extensions, luer locks, suture wings, and the like are useful. In some embodiments, copolymer extrusion is useful in ranges of the second polymer from 0.1% to 10% w/w or no more than 10% w/w of the first polymer, with no more than 5% w/w also being useful. Artisans will immediately appreciate that all ranges and values between the explicitly stated bounds are contemplated, with, e.g., any of the following being available as an upper or lower limit: 0.1, 0.2, 0.4, 0.5, 0.8, 1, 2, 3, 4, 5, 6, 8, 10% w/w.

In some embodiments, salts are useful to manipulate the strength of the materials. Without being limited to a particular theory, it is likely the salts are part of the physical crosslinking, in effect acting as small molecular weight crosslinkers between the polymer chains.

Some embodiments for polymer blends include at least one first hydrophilic polymer and at least one second hydrophilic polymer in a solvent that is extruded as described herein. Examples include combinations of one or more of PVA, PAA, PEG, PVP, polyalkylene glycols, a hydrophilic polymer, and combinations thereof. Examples of concentrations include the at least one second hydrophilic polymer being present at 1 part to 10,000 parts of the first hydrophilic polymer. Artisans will immediately appreciate that all ranges and values between the explicitly stated bounds are contemplated, with, e.g., any of the following being available as an upper or lower limit: 1, 2, 10, 100, 1000, 1500, 2000, 2500, 3000, 4000, 5000, 6000, 7000, 8000, 9000, 10000 parts. Examples of concentrations of polymers in a polymer-solvent mixture include a first polymer present at a first concentration and one or more further polymers present at a second concentration, with the first polymer concentration and the further polymer concentration being independently selected from 0.1-99%, e.g., 0.1, 0.2, 0.3, 0.4, 0.5, 0.6, 1, 2, 3, 4, 5, 6, 7, 8, 9, 10, 11, 12, 13, 14, 15, 16, 17, 18, 19, 20, 25, 30, 33, 35, 40, 45, 50 55, 60, 65, 70, 75, 80, 85, 90, 95% w/w. Further, non-hydrophilic polymers and/or non-hydrophilic blocks in block polymers, may be present, with concentrations of such polymers and/or such blocks generally being less than about 10% w/w, e.g., 0.1, 0.2, 1, 2, 3, 4, 5, 6, 7, 8, 9, 10% w/w.

Some embodiments include porous matrices conditioned with water-soluble polymers that lose no more than 20-90% w/w of the water-soluble polymer under comparable conditions; Artisans will immediately appreciate that all ranges and values between the explicitly stated bounds are contemplated, e.g., 20, 25, 30, 33, 40, 50, 60, 70, 80, 90% w/w.

In some embodiments, bulk incorporated materials may present a monolayer at the surface. The term monolayer means a layer that is a single molecule thick. The monolayer does not rely on cohesion between the molecules of the monolayer to remain stably present at the surface. At least one water-soluble polymer forms the monolayer. In contrast, even a thin polymer coating that is cross-linked to itself has a thickness corresponding to the thickness of the network formed by the cross-linked polymers. For example, it may be possible to create a cross-linked PVA coating on a surface but such a coating relies on interconnections between molecules of the PVA and necessarily forms a crosslinked network. Accordingly, embodiments include a water-soluble polymer present on a surface of a porous solid without covalent bonding to the surface and without the polymer being part of a network.

Artisans reading this disclosure will be able to adapt its principles in light of what is known about extrusion or other forming arts to make alternative processes and devices that achieve the same end products as described herein. A scaled-up embodiment of this process may be adapted for use with, for example, a multi-zone screw extruder, with the solvent mixture provided via a suitable injector or a hopper and the zones controlled to provide a cold extrusion. Features such as the syringe pump can be replaced by a suitably metered and controlled liquid or solid polymer feed system.

As described elsewhere herein, the disclosure provides guidance for bonding a body portion to one or more components. In some embodiments, the body portion comprising a polymer is softened using heat and placed into contact with the one or more components under an applied pressure. Alternatively, or additionally, the component is softened and placed into contact with the body portion under an applied pressure. In other embodiments, body portion (or component) is softened using a solvent comprising a water-soluble polymer and placed into contact with a component (or body portion) under an applied pressure. Without wishing to be bound by any particular theory, it is generally believed that softening the body portion (or component), softens the polymers, which in the presence of an applied pressure, favors polymer chain interdiffusion at the bonding junction, thus forming an integrated article.

In some embodiments, bonding may be performed using body portions and/or components that are desolvated (e.g., dehydrated or dry state, EWC less than or equal to 5% w/w), partially hydrated state, or completely hydrated state (EWC is about 50% w/w). In a preferred set of embodiments, bonding is performed in the desolvated state.

Without wishing to be bound by any particular theory, it is generally believed that heating the bonding junction between the body portion and/or component while in the desolvated state produces a polymer melt at the bonding junction (e.g., the polymers are above the glass transition temperature but below the melting temperature). Applying pressure at the bonding junction permits polymers chains from the body portion to flow freely into the component, and polymers from the component to flow freely into the body portion. Upon cooling, the polymer chains of the body portion are physically entangled and integrated within the bulk of the component and the polymer chains of the component become physically entangled and integrated within the bulk of the body portion. For example, in some cases, the polymer chains of the component may become entangled within the porous bulk structure of the body portion.

Bulk incorporation not only modifies the surface of the body portion and/or component, but also below the surface, e.g., at least, or in a range of, 1-5000 μm; Artisans will immediately appreciate that all ranges and values between the explicitly stated bounds are contemplated, e.g., 1, 2, 3, 4, 5, 10, 20, 30, 40, 50, 75, 100, 250, 500, 750, 1000, 2000, 3000, 4000, or 5000 μm.

Alternatively, or additionally, the bonding junction may be softened using a solvent comprising a water-soluble polymer, configured to swell or rehydrate the porous body portion and/or component. For example, a body portion (e.g., porous hydrophilic material) may be exposed to a solvent comprising solvated polymers (e.g., poly(vinyl alcohol) to draw them into the pores when the porous matrix is desolvated. The solvent has an affinity for the matrix and is drawn in as the matrix imbibes the solvent. The solvent in the mixture with the bulk incorporated polymers can be chosen to have an affinity for the matrix so that it is imbibed into the desolvated matrix but does not have to be the same as the solvent in the matrix. In general, a hydrophilic solvent in the mixture will be imbibed into a hydrophilic porous matrix that is at least partially desolvated and contains a hydrophilic solvent, and an artisan can adjust the various solvents as needed to create suitable conditions when the goal of bulk incorporation is intended.

A hydrophilic solvent is a solvent that is freely miscible with water or is present at a concentration in the mixture wherein it is freely miscible with water, at 20° C.

Desolvated means that the matrix is free of solvents, e.g., completely dry, or is below an EWC of the matrix relative to the solvent it contains. If the solvent in the matrix is not water, the EWC can be calculated for the material based on measurements in the solvent, i.e., the term EWC can be used for solvents that are not water in the appropriate context. For instance, a hydrophilic matrix might be solvated in an aqueous solution of an alcohol and would have an EWC for that solvent. Embodiments include an amount of desolvation of a porous solid from 1-100, Artisans will immediately appreciate that all ranges and values between the explicitly stated bounds are contemplated: 1, 5, 10, 15, 20, 33, 40, 50, 60, 70, 80, 90, 95, 99, 100% w/w referring to the total weight of solvent that can be removed.

Without being bound to a particular theory, it is believed that porous materials can be desolvated (dehydrated in the case of water being the solvent in the porous material) and exposed to polymers in a solution that resolvates the porous material so that the polymers are drawn into the pores. The polymers then form physical bonds with the matrix material that defines the pores and are, for practical purposes, permanently incorporated into the bulk of the materials, both by at least partially filling the pores and by physical bonding with the matrix. Alternatively, or additionally, the polymers have a hydrodynamic radius that causes the polymer to present a diameter that exceeds the pores' opening diameter so that the polymer is permanently incorporated into the pores of the material, especially when the material is to be used in water or physiological solution. In general, if the bulk-incorporated polymer is solvated in a polymer that wets the pores of the porous solid, the polymer can be drawn into pores of the matrix as it is resolvated. When a hydrophilic porous matrix is below an EWC of the matrix, the mixture that contains the polymers for bulk incorporation is drawn in because the solvent for the polymers is matched to the matrix material, e.g., wets the pores of the material. For instance, a hydrophilic solvent will normally wet the pores of a hydrophilic matrix.

A material that comprises a porous matrix of polymers joined by noncovalent bonds is a preferred embodiment, since these materials can be made with a high degree of control over pore sizes and material properties, including a choice of nanoporous, microporous, or other characteristic pore sizes. The matrix may comprise physically crosslinked water-soluble polymers that define the pores. A solids concentration of these water-soluble polymers may be at least 33% w/w of the matrix at an equilibrium water content (EWC) of the matrix, although other concentrations may also be used.

Accordingly, an embodiment of a process of incorporating polymers in a porous material comprises providing a material comprising a porous, hydrophilic matrix that comprises one or more water-soluble polymers (also referred to herein as matrix polymers) physically crosslinked with each other to form the matrix. The material with the matrix is exposed to a solvent comprising one or more polymers (also referred to as bulk incorporated polymers, preferably with the polymers being water-soluble, with the solvent also being referred to as a conditioning mixture or bulk incorporating mixture) solvated in a solvent, wherein the matrix is below the EWC before being exposed to the solvent and is hydrophilic relative to the solvent. The material, before exposure to the solvent with the bulk incorporated polymers, is desolvated.

In some embodiments, bulk incorporation processes create an outer zone wherein the pores are filled, an intermediate zone where most of the pores are filled or are mostly filled, and an inner zone where there is little or no penetration of the polymers. Bulk incorporation not only modifies pores at a surface but also below the surface, e.g., at least, or in a range of, 1-5000 μm; Artisans will immediately appreciate that all ranges and values between the explicitly stated bounds are contemplated, e.g., 1, 2, 3, 4, 5, 10, 20, 30, 40, 50, 75, 100, 250, 500, 750, 1000, 2000, 3000, 4000, or 5000 μm. The percentage of pores that have polymer may be assayed as already described and penetration graded by a cut-off of a percentage, e.g., a first zone having 100% filling of pores, a second zone with 50% pores filled, a third zone with 0% pores filled.

Bulk incorporation processes are preferably made with porous matrices that are made of water-soluble polymers and may be made without hydrophobic domains in the polymers, e.g., a matrix made only of PVA. The polymers may form the matrix with physical crosslinks. Accordingly, embodiments include materials comprising matrices that are free of hydrophobic domains or that are made with water-soluble polymers that are free of hydrophobic domains or that are free of any polymer that is not water-soluble. Some hydrophobic domains can be tolerated, however, when making a hydrophilic matrix with water-soluble polymers having physical crosslinks without disrupting the matrix formed thereby. Embodiments of the invention include a hydrophobic content of polymers that form a porous matrix of 0, 1, 2, 3, 4, 5, 7, 8, 9, 10, 11, 12, or 15% w/w.

A porous matrix consisting essentially of water-soluble polymers refers to a content of up to 3% w/w of the polymers that crosslink to form the matrix. RO agents such as salts are not polymers that crosslink to form the matrix. A porous matrix consisting essentially of physically crosslinked polymers refers to a matrix that is free of agents that make covalent bonds between the polymers, or has a small amount of such agents so that no more than about 6% of the polymers (referring to polymer number) are crosslinked to each other with such agents, e.g., wherein a stoichiometric ratio of polymer number to a bifunctional crosslinker is at least 100:3. A matrix that is essentially free of covalent bonds similarly is made with polymers crosslinked with no more than about 6% of the polymers (by number) are not covalently crosslinked. The number of covalent bonds in a matrix may similarly be limited to a stoichiometric ratio of 100:3 to 100:100, e.g., 100 to any of 3, 5, 10, 15, 20, 25, 30, 40, 50, 60, 70, 80, 90, or 100 by number. For instance, hydrogels made by free radical polymerization typically have 100% of the polymers attached to each other by covalent bonds, which is a 100:100 stoichiometric ratio of polymers:covalent bonds.

As stated elsewhere, a porous solid can be made with a controlled pore diameter range and may be made to provide a matrix that has no pores larger than a particular diameter. Diameters may be measured in an appropriate context, e.g., at EWC in distilled water. Embodiments thus include polymers entrapped in a porous matrix that is free of pores that are larger than 1-5000 μm; Artisans will immediately appreciate that all ranges and values between the explicitly stated bounds are contemplated, e.g., 1, 2, 3, 4, 5, 10, 15, 20, 25, 30, 50, 100, 200, 250, 300, 400, 500, 750, 1000, 2000, 3000, 4000, or 5000 μm.

A porous solid can have other materials present as described elsewhere herein, e.g., radiopaque (RO) agents that are additional to the matrix but are not part of the matrix. RO agents typically contribute little to the crosslinking that provides the strength of the matrix. Similarly, other materials can be present in the matrix without being part of the matrix, e.g., wires and reinforcing materials. It can be appreciated that a matrix made with physical crosslinks is one type of matrix that can be made with materials that define pores that have diameters and is in contrast to hydrogels having polymer strands that are generally separated from each other and are connected in a mesh network structure, e.g., as typically formed using free radical polymerization or by reaction of monomers/polymers that are in solution. Such mesh networks would generally not be expected to stably incorporate polymers in their pores without covalent bonding using a polymer-imbibing process. Porous materials are described in detail herein and these may be freely chosen, as guided by the disclosure herein, for use with bulk incorporated polymers. The porous material may be chosen with bulk properties as described herein.

The bulk incorporated polymers may be polymers described elsewhere herein for porous solids. Examples are water-soluble polymers. The water-soluble polymers may be, for example, polyvinyl alcohol (PVA), polyvinylpyrrolidone (PVP), polyethylene glycol (PEG), polyacrylic acid (PAA), polyacrylamide, hydroxypropyl methacrylamide, polyoxazolines, polyphosphates, polyphosphazenes, poly(vinyl acetate), polypropylene glycol, Poly(N-isopropylacrylamide) (PNIPAM), polysaccharides, sulfonated hydrophilic polymers (e.g., sulfonated polyphenylene oxide, Nafion®, sulfobetaine methacrylate) and variations of the same with an added iodine (e.g., PVA-I, PVP-I), or variations with further pendent groups, copolymers of the same, and combinations of the same. The solvent may comprise one or more polymers, meaning polymers of different chemical compositions, such as PVA and PEG. The term “a polymer” refers to one or more polymers.

The solubility of a water-soluble polymer for a porous matrix or for bulk incorporation may be chosen as, e.g., at least 1, 2, 5, or 10 g/100 ml in water at 20° C. Polymers may be chosen to be linear or branched. Embodiments include a polymer or a hydrophilic polymer having a molecular weight of, e.g., 40k to 5000k Daltons; Artisans will immediately appreciate that all ranges and values between the explicitly stated bounds are contemplated, with, e.g., any of the following being available as an upper or lower limit: 40k, 50k, 100k, 125k, 150k, 250k, 400k, 500k, 600k 750k, 800, 900k, 1 million, 1.5 million, 2 million, 2.5 million, 3 million molecular weight. The molecular weight of the polymer can be chosen in light of the pore sizes available in the porous solid. Nanoporous or microporous materials are preferred.

The bulk incorporated polymers may be chosen to be the same as polymers that form the porous matrix, to be the same as at least one of the polymers that make up the matrix, or to be different.

The bulk incorporated polymer concentrations in the solvent may be, referring to the solvent at the start of the process, any concentration wherein the polymers go into solution, bearing in mind that polymer that is not in solution, or other non-solvated materials, are not destined to enter pores. In some embodiments, concentrations are 1-50% w/w; Artisans will immediately appreciate that all ranges and values between the explicitly stated bounds are contemplated, e.g., 1, 2, 3, 4, 5, 10, 15, 20, 25, 30, 33, 35, 40, 50% w/w.

Solvents may be chosen as appropriate to solvate the polymer and to provide a solvent that will be imbibed by the porous solid. Hydrophilic solvents are generally preferable for a hydrophilic matrix. Solvents may be water, organic, or aqueous, or free of the same, e.g., free of organic solvent. In some embodiments, concentrations of water are 0-99, e.g., 0, 5, 10, 15, 20, 25, 30, 40, 50, 60, 70, 80, 90, 95, or 99 w/w %.

A temperature of the conditioning mixture is not to exceed a melting temperature of the porous solid matrix. Temperatures ranges may be, for example, from 10-100° C., e.g., 10, 20, 30, 37, 40, 50, 60, 70, 80, or 90° C.

Exposure times are preferably for a duration of time required for a porous solid to reach EWC in the mixture. Duration of time may comprise, in some embodiments, 2, 4, 6, 8, 10, 12, 16, 20, 24, and 48 hours. Agitation and temperature may be manipulated to affect a time of exposure, e.g., to accelerate achieving EWC or to control viscosity of the mixture. Salt and/or osmotic content may be adjusted as helpful, e.g., for solubility, viscosity, and/or EWC.

The Examples provide guidance in regards to salt concentration for a conditioning mixture. Examples of salt concentration are from 0.1 to 2% w/w. In general, a single charge cation with a smaller atomic radius has a greater penetration into a depth of a porous solid, whereas a larger cation reduces penetration. Examples of salts are those with a single cation, divalent cation, or other cation, e.g., a salt of sodium, potassium, lithium, copper, quaternary ammonium

$$( {NR}_{4}^{+} , where R is a hydrogen , alkyl , or aryl group ) ,$$

magnesium, calcium, copper, iron, or zinc. In general, a physiological pH using a buffer was useful for the mixture. A pH may be adjusted to increase or decrease penetration into a matrix, and the solvent may include or omit buffering salts. Examples of pH are from 4-10, e.g., 4, 5, 6, 7, 8, 9, or 10.

A viscosity of a conditioning mixture, referring to a water-soluble polymer and solvent, is affected by: pH (higher pH, higher viscosity), polymer concentration and/or molecular weight, and polymer branching, with increases in any of these generally leading to a higher viscosity. In general, a higher viscosity reduces penetration of the bulk incorporating polymers into a porous solid. An embodiment is a porous material comprising water-soluble polymers entrapped in pores of a porous matrix. The matrix may comprise physically crosslinked water-soluble polymers that are crosslinked with each other to form the matrix and define the pores. The matrix may have features as disclosed herein, e.g., polymer content, weight percentage of polymers, strength, Young's modulus, degree of coverage, pore sizes, and so forth.

Surface coverage of the water-soluble polymers in a porous matrix may be complete. Complete coverage under SEM conditions wherein no pores of the underlying surface are visible indicates coverage at EWC. A degree of coverage may be less than 100%, e.g., from 50-100%; Artisans will immediately appreciate that all ranges and values between the explicitly stated bounds are contemplated, e.g., 50, 60, 70, 80, 90, 95, 98, 99, 99.9, or 100%.

Bulk incorporation can decrease physical properties of a porous solid. Embodiments thus include a porous solid, e.g., one as disclosed herein, with a Young's modulus and/or tensile strength that is from 1-20% less as a result of being conditioned with a water-soluble polymer as compared to the same material that has not been conditioned with a water-soluble polymer; Artisans will immediately appreciate that all ranges and values between the explicitly stated bounds are contemplated, e.g., 1, 2, 3, 4, 5, 7, 9, 10, 12, 15, or 20%. A test for incorporation of water-soluble polymers to be stable is: Immersion of the test device in physiologically representative fluid (i.e. PBS) at body temp conditions in a circulating peristaltic loop with the test device placed directly in the head of the pump at a flow rate of 10-12 mL/s for 24 hrs at 150 rpm, approximating 500,000 mechanical sample compressions with a volume flux rate of 0.1225 cm³*s⁻¹*cm⁻². While testing revealed as much as a 25% loss, other test criteria may be used, e.g., a loss of 0-50% w/w, e.g., 1, 5, 10, 15, 20, 25, 30, 40, 50% w/w. Or other tests may be posed, e.g., a loss of 0-5% w/w e.g., 1, 2, 3, 4, or 5% w/w at 1-52 weeks of static exposure to an excess of PBS, e.g., 1, 2, 3, 4, 5, 10, 20, 30, 40, 50, or 52 weeks.

The present disclosure relates in some aspects to porous materials (e.g., porous hydrophilic materials, body portion, etc.). Processes are provided herein to create biocompatible porous solids such as microporous or nanoporous solid materials that possess low protein adsorption properties and provide a basis for non-biofouling devices. Modification of starting polymer concentration, molecular weight, solvent removal, forming processes, and hardening/annealing processes may be utilized to provide surface properties with reduced protein adsorption and other properties. Some embodiments include creation of various continuous shapes through extrusion of a polymeric mixture. The mixture may be further hardened and annealed. These processes may be used to create a tough and highly lubricious material. Embodiments include polymeric mixtures extruded into shapes possessing single or multiple lumens, of varied diameters and wall thickness.

An embodiment of a process for making a nanoporous solid material comprises heating a mixture that comprises a polymer and a solvent (a polymeric mixture), extruding the mixture into a solvent-removing environment, and removing the solvent from the crosslinked matrix until a nanoporous solid material is formed. One or more of these actions may be combined, depending on the process. Further, cooling the mixture as it passes out of the die is useful. Without being bound to a specific theory of operation, it appears that crosslinking the polymer during passage through the die initially forms a porous matrix that is not a true nanoporous solid material because, although it has spaces between polymer strands, it does not have a pore-structure. As the solvent is removed under appropriate conditions, the crosslinked structure becomes a nanoporous solid. The crosslinking starts when the polymeric mixture is extruded through a die, and as the mixture is cooled. The crosslinking may continue while the solvent is removed. The transition to form the nanoporous material takes place as the solvent is removed and, in general, is believed to be completed or essentially completed (meaning 90% or more) at this stage. The resultant material may be further processed by annealing with or without a presence of further solvents, or plasticizers. This process, and the other extrusion or other formation processes and/or materials set forth herein, including bulk incorporation processes, may be free of one or more of: covalent crosslinking agents, agents that promote covalent crosslinks, radiation that crosslinks polymer chains, freezing, thawing, freeze-thaw cycles, more than one freeze-thaw cycle, ice-crystal formation, foaming agents, surfactants, hydrophobic polymers, hydrophobic polymer segments, reinforcing materials, wires, braids, non-porous solids, and fibers.

The porous materials may be made by an extrusion process that comprises passing a polymeric mixture through a die into a cooling environment. The cooling environment may further be a solvent-removing environment. It is a dehydrating environment when the solvent is water. The die may have a core that passes through it so that the polymeric mixture may be formed around the core. Further solvent-removal environments and/or annealing environments may be used.

The extrusion process for a polymer-solvent mixture may be performed as a cold extrusion. The term cold extrusion refers to a process that involves passing a polymer-solvent mixture through a die and does not require heating the polymer-solvent mixture above its boiling point during the entire process of preparing the polymer-solvent mixture and extruding it.

Accordingly, in a cold extrusion, the die head is kept below a boiling point of the polymer-solvent mixture. Although many solvents may be used, water is often a useful solvent in which case the die head is kept at 100° C. or less, although colder temperatures may be useful, as discussed above.

The term polymeric mixture refers to a polymer that is in solution, dissolved, or suspended in a solvent. A solvent may be, e.g., water, aqueous solution, an organic solvent, or combinations thereof. Heating the polymeric mixture may comprise heating the mixture to a temperature above the melting point of the polymer. In general, the solution transitions from a cloudy to a clear state when it reaches the melt point. An aqueous solution contains water, for instance from 10-100% (w/w or v/v) of the liquid being water; Artisans will immediately appreciate that all ranges and values between the explicitly stated bounds are contemplated, e.g., 10, 20, 30, 40, 50 60, 70, 80, or 90% or at least one of the same.

Extrusion is a useful process for forming the materials. Other forming processes may be used, for example, molding, casting, or thermal forming polymer-solvent mixtures. In general, a polymer-solvent mixture is prepared without boiling and formed into a shape that is exposed to solvent-removal conditions that are controlled to make a nanoporous or microporous material using the guidance provided herein. An annealing process may be included. Hydrogels that are not microporous or nanoporous materials can also be made.

The heated polymeric mixture may be molded or otherwise formed as it is cooled or molded/formed and immediately cooled. Formed is a broad term that refers to passing the material from an amorphous melted state into an end-user product or an intermediate shape for further processing. Forming encompasses casting, layering, coating, injection molding, drawing, and extrusion. The forming can be done using an injection molding set up, where the mold consists of a material with thermal conductive properties allowing it to be heated easily to enhance the flow of the injected polymeric mixture, and to be cooled rapidly in a cooling environment. In other embodiments, the molding process can be accomplished by extrusion of the polymeric mixture through a die to form continuous material.

Cooling the polymeric mixture may comprise, e.g., cooling an extruded material, as in the case of passing the polymeric material through a die. An embodiment for cooling is a liquid bath at a temperature at least 20° C. cooler than the polymeric mixture boiling point or alternatively below the polymeric mixture Tm, e.g., 20, 30, 40, 50, 60, 70, 80, 90, 100, 110° C. below the boiling point or polymeric Tm, or alternatively the bath or other environment being at a temperature from −50 to 30° C.; Artisans will immediately appreciate that all ranges and values between the explicitly stated bounds are contemplated, with, e.g., any of the following being available as an upper or lower limit: −50, −45, −25, −20, −10, −5, −4, 0, 15, 20, 25, 30° C. The cooling may be performed in a solvent removing environment. Freezing temperatures may be avoided. Without being bound to a particular theory of operation, the polymer chains are cooled to the point of promoting intermolecular hydrogen binding and immobilizing chain movement.

This may occur at temperatures as high as 30° C., or higher if time is allowed. The bath may be aqueous, and, by adjustment with salt or other osmotic agents, may be provided at an osmotic value to perform solvent removal on aqueous materials that are at a relatively lower osmotic value through osmotic pressure and diffusion. The bath may also be other solvents that freeze at temperatures lower than water, so that temperatures below 0° C. may be used without freezing the solvent or materials. In the event that hydrophilic copolymers are used in conjunction with PVA, for instance, temperatures higher than 20° C. may be used as crosslinking and chain immobilization will occur at much higher temperatures.

A solvent-removing environment refers to an environment that significantly accelerates removal of a solvent as compared to drying at ambient conditions. Such an environment may be non-heating, meaning it is not above ambient temperature, e.g., not above 20° C. Such an environment may be a vacuum, e.g., a vacuum chamber, a salt bath, or a bath that removes the solvent in the polymeric mixture. For instance, an aqueous polymeric mixture may be introduced into an ethanol bath, with the ethanol replacing the water. The ethanol may subsequently be removed. A salt bath may be, e.g., a high salt concentration bath (1M to 6M).

A time of processing in a solvent-removing environment and/or a cooling process may be independently chosen to be from 1 to 240 hours; Artisans will immediately appreciate that all ranges and values between the explicitly stated bounds are contemplated, with, e.g., any of the following being available as an upper or lower limit: 1, 2, 5, 10, 24 hours, 1, 2, 5, 7, 10 days. Salts may be salts that dissociate to make single, double, or triply charged ions.

One or a plurality of solvent-removing environments may be used, or one environment may be adjusted with respect to temperature. Thus, a cooling bath may be used followed by solvent removal in an oven or vacuum oven. A washing step may be performed before or after cooling or solvent removal, e.g., by soaking in a series of solvents of varying concentrations, varying salt solutions, varying proportions of ethanol or other solvents.

An embodiment is an extruded material that has been through a solvent-removal process comprising exposure to a salt bath, the material is soaked in a series of H₂O baths (new baths or exchanged) for a period of time (e.g., 2-48 hours, 4-24 hours) to remove excess salt from the cast material or end-user device. The material is removed from the wash step and dehydrated to remove excess water. Dehydration can be done using, e.g., temperatures ranging from 20-95° C. Dehydration is generally performed at 37° C. for greater than 24 hours.

An embodiment is a polymeric mixture that has been extruded or otherwise formed that is then exposed to a high salt concentration bath (1M to 6M) for an inversely correlated period of time; high salt reduces the time required for soaking; for instance, it is soaked for 16-24 hours in a 6M solution of NaCl. After soaking, the material is rinsed free of salt solution. The material is now toughened and can be removed from any mold pieces carried over from the initial formation. Alternatively, after a salt or other bath, the material is soaked in water baths and dehydrated to remove excess water. Dehydration can be done using temps ranging from 20-95° C. Dehydration may be performed at 37° C. for greater than 4 hours, greater than 24 hours, or in a range from 2 to 150 hours; Artisans will immediately appreciate that all ranges and values between the explicitly stated bounds are contemplated, with, e.g., any of the following being available as an upper or lower limit: 2, 4, 6, 8, 10, 12, 16, 24, 48, 72, 96, 120, 144, 150 hours. For instance, dehydration at 40° C. for 6-24 hours has been observed to be useful.

In another embodiment, NaCl is incorporated into the starting polymeric solution at concentrations ranging from 0.1 to 3M of the final polymeric mixture volume. A polymer is dissolved in a heated solution under agitation, then brought above its melt point. To this solution, dry NaCl is added slowly under agitation until completely dissolved. The slightly hazy solution is then drawn into a feed for the purpose of creating a shape, either through injection molding, casting, extrusion and/or drawing. A quench is performed at the end of each process to rapidly reduce the temperature and form a solid material. In this embodiment, no additional salt soak is required. After material hardening, if necessary, the material is removed from any molding process parts and rinsed in water to remove salt and dehydrated.

The term annealing, as used in the context of a semi-crystalline polymer or a solid porous material refers to a heat treatment at an annealing temperature comparable to the melting temperature of the polymer or the polymers in the relevant material. This temperature is usually less than and is within about 0-15% of the melting temperature on an absolute temperature scale. Plasticizers or other additive materials may affect the melting temperature, usually by depressing it. For a pure PVA, for instance, the annealing temperature will be within about 10% of the melting point of the PVA; with other materials present, the annealing temperature will typically be lower. A theory of operation is that the annealing is a process that is a relaxation of stress combined with increase in the size of crystalline regions in the material being annealed. Unlike metals, annealing increases the strength of the annealed material. Annealing may be performed in one or more of: in air or in a gas or in an absence of oxygen or an absence of water, e.g., in nitrogen, in vacuum nitrogen, under argon, with oxygen scavengers, and so forth. For example, experiments have been made with annealing dehydrated PVA nanoporous materials. Annealing is utilized to increase crystallinity in the PVA network, further reducing pore sizes of the PVA network and to reduce adsorption properties of the final gel surface. Annealing can be done at temperatures ranging from, e.g., 100-200° C.; in a preferred embodiment, this step is performed submerging the dehydrated gel into a bath of mineral oil. Bulk incorporation of a polymer into a porous solid may also include an annealing process as already described above for a porous solid. Annealing may be performed after exposure of the desolvated porous solid to the mixture that has the polymers that are to be bulk incorporated. The Tg of the material may be raised or lowered dependent on the residual solvent content and/or presence of the bulk incorporated second hydrophilic polymer. As already described, the annealing process conditions may thus be adapted as to depend on temperature, time, ramp rate, and cooling rates of the substrate.

Annealing may be performed in a gas or a liquid at ambient, elevated, or low (vacuum) pressure. The liquid may be a low molecular weight polymer (up to 2000 Da) or other material (e.g., mineral oil). Examples of low molecular weight polymers are: silicone oils, glycerin, polyols, and polyethylene glycols of less than 500 Da. A useful embodiment is annealing in a bath of glycerin at, e.g., 140° C. for 1-3 hours; glycerin acts to further reduce fouling properties of the gel through interaction and neutralization of the free hydroxyl end groups of the PVA network. The annealed nanoporous material is allowed to cool, removed from the annealing bath and rinsed free of bath medium using a series of extended soaks. The product is then dehydrated to prepare for terminal sterilization.

Various types of dies may be used, e.g., longitudinal, angular, transverse and spiral extrusion heads, as well as single-polymer extrusion heads used for extruding a single polymer and multi layers extrusion heads used for simultaneous extrusion of a plurality of polymer layers or other layers. Continuous operation heads may be used, as well as cyclical. Various materials may be incorporated into, or as, a layer: for example, a reinforcing material, a fiber, a wire, a braided material, braided wire, braided plastic fibers, and so forth. Similarly, such materials may be excluded. Moreover, the porous solid may be made with a certain property, e.g., Young's modulus, tensile strength, solids content, polymer composition, porous structure, or solvent content that is known and thus measurable exclusive of various other materials. Accordingly, embodiments include materials disclosed herein that are described in terms of the materials' properties without regard to various other incorporated materials. For instance, a nanoporous solid has a certain Young's modulus that is known even if the material has a reinforcing wire that contributes further strength.

A core may be used with an extrusion die. The core may be air, water, a liquid, a solid, a non-solvent or a gas. Artisans reading this disclosure will appreciate that various extrusion processes using these various kinds of cores may be used. Cores made of polytetrafluoroethylene tubing (PTFE) are useful. In some embodiments, a core is a wire.

Multi lumen tubing has multiple channels running through its profile. These extrusions can be custom engineered to meet device designs. Multi Lumen tubing has a variable Outer Diameter (OD), numerous custom Inner Diameters (ID's), and various wall thicknesses. This tubing is available in a number of shapes; circular, oval, triangular, square, semi-circular, and crescent. These lumens can be used for guidewires, fluids, gases, wires, and various other needs. The number of lumens in multi lumen tubing is only limited by the size of the OD. In some embodiments, OD's are as large as 0.5 in., ID's can be as small as 0.002 in., and web and wall thicknesses can be as thin as 0.002 in. Tight tolerances can be maintained to +1-0.0005 in. Artisans will immediately appreciate that all ranges and values between the explicitly stated bounds are contemplated, with, e.g., any of the following being available as an upper or lower limit for an OD and/or ID: 0.002, 0.003, 0.004, 0.007, 0.01, 0.02, 0.03, 0.04, 0.05, 0.1, 0.2, 0.3, 0.4, and 0.5 in. Tolerances may be, e.g., from 0.0005 to 0.1 in.; Artisans will immediately appreciate that all ranges and values between the explicitly stated bounds are contemplated, with, e.g., any of the following being available as an upper or lower limit: 0.0005, 0.001, 0.002, 0.003, 0.006, 0.01, 0.02, 0.03, 0.06, 0.8, 0.9, 1 in.

Braid reinforced tubing can be made in various configurations. For instance, it is possible to braid using round or flat, single or double ended wires as small as 0.001 in. Various materials can be used to make the braided reinforced tubing including stainless steel, beryllium copper, and silver, as well as monofilament polymers. The braid can be wound with various pics per inch over many thermoplastic substrates such as nylons or polyurethanes. The benefits of braided catheter shaft are its high torque-ability and kink resistance. By changing several factors during the braiding process, the characteristics of the tube can be altered to fit performance requirements. After braiding is complete, a second extrusion may be applied on top of the braided tube to encapsulate the braid and provide a smooth finish. Walls as thin as 0.007 in. can be achieved when a braid reinforced tube is required.

The devices, catheters, kits, and methods described herein may be administered to any suitable subject. The term “subject”, as used herein, refers to an individual organism such as a human or an animal. In some embodiments, the subject is a mammal (e.g., a human, a non-human primate, or a non-human mammal), a vertebrate, a laboratory animal, a domesticated animal, an agricultural animal, or a companion animal. Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon administration of the device.

Products, including end-user or intermediate products, or materials, may be made that have an aspect ratio as desired, e.g., at least 3:1, referring to materials set forth herein including nanoporous materials, microporous materials, and hydrogels. The aspect ratio increases as the device increases in length and decreases in width. Artisans will immediately appreciate that all ranges and values between the explicitly stated bounds are contemplated, with, e.g., any of the following being available as an upper or lower limit: 3:1, 4:1, 5:1, 6:1., 7:1, 8:1., 9:1, 10:1, 50:1, 100:1, 1000:1. A high aspect ratio is highly advantageous for certain devices, e.g., many types of catheters. In principle, a thin tube could be continuously extruded without limitation as to length. Such devices include, e.g., tubes, rods, cylinders, and cross-sections with square, polygonal, or round profiles. One or more lumens may be provided in any of the same. The devices may be made of a single material, essentially a single material, or with a plurality of materials including the various layers already discussed, or a reinforcing material, a fiber, a wire, a braided material, braided wire, braided plastic fibers.

The extrusion process, in particular, provides for concentric placement of a lumen; concentric is in contrast to eccentric meaning the lumen is off-center. In the case of a plurality of lumens, the lumens may be placed so that the lumens are symmetrically placed: the symmetry is in contrast to an eccentric placement of the lumens that is a result of a poorly controlled process. Embodiments include the aforementioned devices with an aspect ratio of at least 3:1 with lumens that are positioned without eccentricity or one lumen that is concentric with the longitudinal axis of the device.

The porous solids such as the nanoporous materials, microporous materials, and strong hydrogels may be used to make catheters or medical fibers. These may be made with bulk incorporated polymers and may have the various features described for the same. Examples of catheters are central venous, hubbed catheters, peripherally inserted central, midline, peripheral, tunneled, dialysis access, hemodialysis, vascular access port, peritoneal dialysis, urinary, neurological, peritoneal, intra-aortic balloon pump, diagnostic, interventional, drug delivery, etc.), shunts, wound drains (external including ventricular, ventriculoperitoneal, and lumboperitoneal), and infusion ports. The porous solids may be used to make implantable devices, including fully implantable and percutaneously implanted, either permanent or temporary. The porous solid materials may be used to make blood-contacting devices or devices that contact bodily fluids, including ex vivo and/or in vivo devices, and including blood contacting implants. Examples of such devices include drug delivery devices (e.g., insulin pump), tubing, contraceptive devices, feminine hygiene, endoscopes, grafts (including small diameter<6 mm), pacemakers, implantable cardioverter-defibrillators, cardiac resynchronization devices, cardiovascular device leads, ventricular assist devices, catheters (including cochlear implants, endotracheal tubes, tracheostomy tubes, drug delivery ports and tubing, implantable sensors (intravascular, transdermal, intracranial), ventilator pumps, and ophthalmic devices including drug delivery systems. Catheters can comprise a tubular nanoporous material with a fastener to cooperate with other devices, e.g., luer fasteners or fittings. Radiopaque agents may be added to the materials, fibers, or devices. The term radiopaque agent refers to agents commonly used in the medical device industry to add radiopacity to materials, e.g., barium sulfate, bismuth, or tungsten. RO agents may be incorporated at, e.g., from 5-50% w/w pf the total solids weight, e.g., 5, 10, 20, 30, 40, or 50%.

Medical fibers made with porous solid materials include applications such as sutures, yarns, medical textiles, braids, mesh, knitted or woven mesh, nonwoven fabrics, and devices based on the same. The fibers are strong and pliable. Materials may be made with these fibers so that they are resistant to fatigue and abrasion.

In some embodiments, the devices described herein are, or are configured for use with, a medical device such as a catheter, a hub, a cuff, a balloon, a shunt, a wound drain, an infusion port, a drug delivery device, a tube, a contraceptive device, a feminine hygiene device, an endoscope, a graft, a pacemaker, an implantable cardioverter-defibrillator, a cardiac resynchronization device, a cardiovascular device lead, a ventricular assist device, an endotracheal tube, a tracheostomy tube, an implantable sensor, a ventilator pump, and an ophthalmic device. In some embodiments, the catheter is selected from the group consisting of central venous catheters, peripheral central catheters, midline catheters, peripheral catheters, tunneled catheters, dialysis access catheters, urinary catheters, neurological catheters, percutaneous transluminal angioplasty catheters and/or peritoneal catheters. Other suitable uses are described in more detail, below.

In some embodiments, the devices and compositions described herein are administered to a subject. In some embodiments, the device may be administered orally, rectally, vaginally, nasally, intravenously, subcutaneously, or uretherally. In some cases, the device may be administered into a cavity, epidural space, vein, artery, orifice, external orifice, and/or abscess of a subject. A non-limiting example of an orifice includes a wound. A non-limiting example of a wound includes a wound orifice that is created for venous access (e.g., created as an insertion site) through the skin.

The term medically acceptable generally refers to a material that is highly purified to be free of contaminants and is nontoxic. The term consists essentially of, as used in the context of a biomaterial or medical device, refers to a material or device that has no more than 3% w/w of other materials or components and said 3% does not make the device unsuited to intended medical uses. Equilibrium water content (EWC) is a term that refers to the water content of a material when the wet weight of the material has become constant, and before the material degrades. In general, materials with a high solids content have been observed to be at equilibrium water content at 24-48 hours. For purposes of measuring EWC, distilled water is used unless otherwise specified.

The term w/v refers to weight per volume e.g., g/L or mg/mL. The terms biomaterial and biomedical material are used interchangeably herein and encompass biomedically acceptable materials directed to a use in the biomedical arts, for example, as implants, catheters, blood-contacting materials, tissue-contacting materials, diagnostic assays, medical kits, tissue sample processing, or other medical purposes. Moreover, while the materials are suited for biomedical uses, they are not limited to the same and may be created as general-purpose materials. A physiological saline refers to a phosphate buffered solution with a pH of 7-7.4 and a human physiological osmolarity at 37° C.

The term molecular weight (MW) is measured in g/mol. The MW of a polymer refers to a weight average MW unless otherwise stated. When the polymer is part of a porous solid, the term MW refers to the polymer before it is crosslinked. When a distance between crosslinks is specified, it is the weight average MW between crosslinks unless otherwise indicated. The abbreviation k stands for thousand, M stands for million, and G stands for billion such that 50k MW refers to 50,000 MW. Daltons is also a unit of MW and likewise refers to a weight average when used for a polymer.

Publications, journal devices, patents and patent applications referenced herein are hereby incorporated herein for all purposes, with the instant specification controlling in case of conflict. Features of embodiments set forth herein may be mixed and matched as guided by the need to make an operable process or product.

As used herein, the term “therapeutic agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition.

As used herein, when a component is referred to as being “adjacent” another component, it can be directly adjacent to (e.g., in contact with) the component, or one or more intervening components also may be present. A component that is “directly adjacent” another component means that no intervening component(s) is present.

A “subject” refers to any animal such as a mammal (e.g., a human). Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon administration of the self-righting device.

As used herein, a “fluid” is given its ordinary meaning, i.e., a liquid or a gas. A fluid cannot maintain a defined shape and will flow during an observable time frame to fill the container in which it is put. Thus, the fluid may have any suitable viscosity that permits flow. If two or more fluids are present, each fluid may be independently selected among essentially any fluids (liquids, gases, and the like) by those of ordinary skill in the art.

### EXAMPLES

### Example 1

The following example generally describes the formation of a catheter tip, in accordance with some embodiments described herein.

### “Soft” Material for the Tip

Tip is fused (glued or thermally bonded) with a lower durometer material to aid in reduction of trauma during insertion. Injection molded or extruded and skived PVA tip annealed/heat treated to a 120° C. is adhered to a PVA tube annealed/heat treated to 150° C. Adherence is achieved using PVA Glue (10% PVA in water), desiccation, and annealing to 120-150° C. Adherence is achieved using radiofrequency or localized heating and water as a solvent to reflow the material at the location of needed adhesion.

### “Hard” Material for the Tip

Tip is fused (glued or thermally bonded) with a higher durometer material to aid in insertion of through the skin, subcutaneous layer, and vascular wall (arterial or venous). Injection molded or extruded and skived PVA tip annealed/heat treated to a 170° C. is adhered to a PVA tube annealed/heat treated to 150° C. Adherence is achieved using PVA Glue (10% PVA in water), desiccation, and annealing to 120-150° C. Adherence is achieved using radiofrequency or localized heating and water as a solvent to reflow the material at the location of needed adhesion.

### Example 2

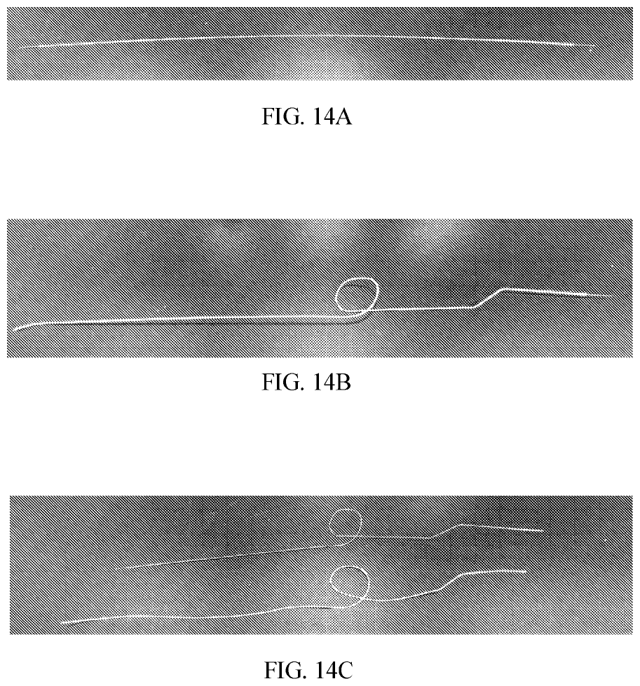

The following example generally relates to forming catheters having shape memory. Shape formation was observed to occur at greater than or equal to 70° C., 90° C., 120° C., 150° C., and 170° C. (with increasing temperature exposure generally providing a stiffer and sharper memory to underlying mandrel shape). Shape memory was achieved using stainless mandrel bent to the specific desired shape, radii, or curvature. Straight or curved catheter tube is extruded and while partially hydrated in a water or salt solution (sodium chloride or phosphate buffered saline) or alcohol/water solution (85% ethanol/15% water) can be soft enough to place on a straight, curved, bent, looped, or corkscrewed to induce shape memory when dried (<90° C.) and then annealed (>90° C.) 120° C. FIGS. 14A-14C are photographs of exemplary shaped catheters. FIG. 14A is a photograph of a partially hydrated straight catheter. FIG. 14B is a photograph of a partially hydrated straight catheter (in 2.2% sodium chloride) on shaped mandrel. FIG. 14C (above) shows the shaped mandrel. FIG. 14C (below) is a photograph of an exemplary catheter dried 95° C., 90m minutes and annealed at 150° C., 90 minutes, rehydrated in 1× phosphate buffered saline 37° C., with the mandrel removed.

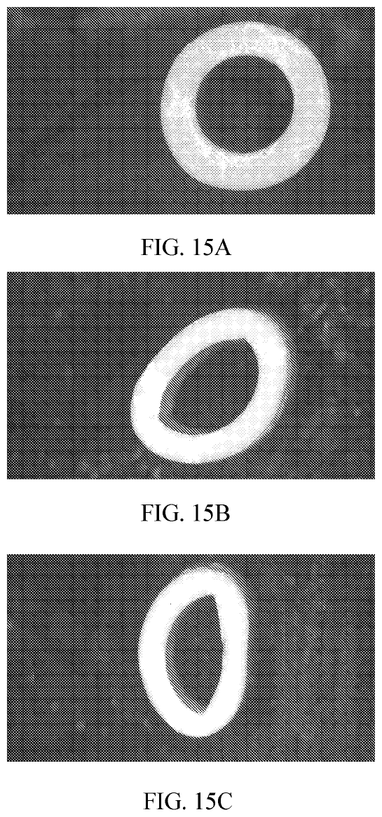

When attempting to reshape a lumen from a circle to a D-shape, hydrating the catheter and drying at varying temperatures imparted aspect ratios similar to the underlying mandrel shape with varying degrees, generally proportional to temperature (see FIGS. 15A-15C).

| | 20 C. | | | |
|---|---|---|---|---|
| | (no change) | 70 C. | 95 C. | Mandrel |
| Aspect Ratio | 1.03 | 1.56 | 2.10 | 2.41 |
| (Height/Width) | | | | |
| % Mandrel Aspect Ratio | 42.8% | 64.7% | 87.1% | N/A |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, device, material, kit, and/or method described herein. In addition, any combination of two or more such features, systems, devices, materials, kits, and/or methods, if such features, systems, devices, materials, kits, and/or methods are not mutually inconsistent, is included within the scope of the present invention.

The indefinite devices “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape, orientation, alignment, and/or geometric relationship of or between, for example, one or more devices, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, circular/circle, rectangular/rectangle, triangular/triangle, cylindrical/cylinder, elliptical/ellipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, east, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated device that would described herein as being “square” would not require such device to have faces or sides that are perfectly planar or linear and that intersect at angles of exactly 90 degrees (indeed, such A device can only exist as a mathematical abstraction), but rather, the shape of such device should be interpreted as approximating a “square,” as defined mathematically, to an extent typically achievable and achieved for the recited fabrication technique as would be understood by those skilled in the art or as specifically described. As another example, two or more fabricated devices that would described herein as being “aligned” would not require such devices to have faces or sides that are perfectly aligned (indeed, such A device can only exist as a mathematical abstraction), but rather, the arrangement of such devices should be interpreted as approximating “aligned,” as defined mathematically, to an extent typically achievable and achieved for the recited fabrication technique as would be understood by those skilled in the art or as specifically described.

What is claimed is:

1. An article, comprising:
a body portion, wherein the body portion comprises a first material comprising a water-soluble polymer; and
a component physically integrated with the body portion, the component comprising a second material different than the first material,
wherein one or more of the following holds:
i) the first material has a Young's elastic modulus of greater than or equal to 500 MPa in a dehydrated state and a Young's elastic modulus of less than or equal to 300 MPa and greater than or equal to 5 MPa at an equilibrium water content state;
ii) the first material is configured to swell in an amount greater than or equal to 5 w/w % and less than or equal to 50 w/w % from a dehydrated state to an equilibrium water content state in less than or equal to 60 minutes at 25° C.;
iii) the first material is free of covalent crosslinks between the water-soluble polymer that forms the first material; and/or
iv) the first material comprises pores that each have a diameter of 1 μm or less.

2. An article, comprising:
a hydrophilic porous polymeric material comprising a water-soluble polymer, the hydrophilic porous polymeric material comprising a plurality of pores, wherein the hydrophilic porous polymeric material is free of covalent crosslinks between the water-soluble polymer; and
a component physically integrated with the hydrophilic porous polymeric material, the component comprising a second material different than the first material,
wherein the component comprises a plurality of fibers, and
wherein at least a portion of the plurality of fibers are embedded within the hydrophilic porous polymeric material.

3. An article, comprising:
a body portion, wherein the body portion comprises a first material comprising a water-soluble polymer, the first material comprising a plurality of pores; and
a component physically integrated with the body portion such that at least a portion of the component is embedded within the body portion, the component comprising a second material different than the first material.

4. A method of forming an integrated article, comprising:
with a polymeric mixture comprising at least one water-soluble polymer and a solvent, the polymeric mixture having a concentration of at least 10% w/w of the at least one water-soluble polymer, performing the steps of:
heating the polymeric mixture to achieve a temperature above the melting point of the polymeric mixture,
extruding the polymeric mixture as a body portion; and
removing the solvent from the extruded body portion at a temperature above a freezing point of the solvent until the body portion is a porous, with the body portion being a porous body portion that comprises the at least one water-soluble polymer, and the porous body portion is made without covalent crosslinking agents that form covalent crosslinks between polymers extruded to make the porous body portion,
wherein, during the step of extruding the polymer mixture, a component is physically integrated with the body portion, the component comprising a second material different than the first material, thereby forming the integrated article.

5. A method of forming an integrated article, comprising:
exposing a body portion to a solvent;
optionally, heating the body portion to a temperature of less than or equal to 100° C., wherein the body portion comprises a first material comprising a water-soluble polymer, such that, in the presence of the solvent, the water-soluble polymer softens;
contacting the body portion with a component in the presence of the solvent, such that the component physically integrates with the body portion thereby forming the integrated article; and
cooling the integrated article.

6. A method of forming an integrated article, comprising:
exposing a body portion to a solvent,
wherein the body portion comprises a first material comprising a first water-soluble polymer, and
wherein the solvent comprises water and a second water-soluble polymer;
contacting the body portion with a component in the presence of the solvent, such that the component physically integrates with the body portion thereby forming the integrated article; and
at least partially drying the integrated article.

7. An article, comprising:
a body portion, wherein the body portion comprises a first material comprising a water-soluble polymer;
a solution coating disposed on a portion of a surface of the body portion, wherein the solution coating comprises water and polyvinyl alcohol, the polyvinyl alcohol present in the solvent in an amount of greater than or equal to 0.1 wt % and less than or equal to 25 wt %; and
a component in physical contact with the solution coating, such that the component is adhered to the body portion via the solution coating.

8. The article or method of any preceding claim, wherein the solvent comprises the water-soluble polymer at a concentration less than the concentration of the water-soluble polymer used to form the first material.

9. The article or method of any preceding claim, wherein the solvent comprises water and polyvinyl alcohol, the polyvinyl alcohol present in the solvent in an amount of greater than or equal to 0.1 wt % and less than or equal to 25 wt %.

10. The article or method of any preceding claim, wherein the solvent has hydrolysis levels of greater than or equal to 80%, such that the solvent comprises less than or equal to 20% acetate groups.

11. The article or method of any preceding claim, wherein the solvent comprises water.

12. The article or method of any preceding claim, wherein the solvent comprises water and the water-soluble polymer.

13. The article as in any preceding claim, wherein the article comprises a lumen.

14. The article as in any preceding claim, wherein the article comprises a hemodialysis catheter, a peripherally inserted central catheter (PICC), a central venous catheter, a midline catheter, a peripheral catheter, a urinary catheter, a neurological catheter, a peritoneal catheter, and/or an infusion port.

15. The article as in any preceding claim, wherein the article is configured for use with a medical article such as a catheter, a balloon, a shunt, a wound drain, an infusion port, a drug delivery article, a tube, a contraceptive article, a feminine hygiene article, an endoscope, a grafts, a pacemaker, an implantable cardioverter-defibrillator, a cardiac resynchronization article, a cardiovascular article lead, a ventricular assist article, an endotracheal tube, a tracheostomy tube, an implantable sensor, a ventilator pump, and an ophthalmic article

16. The article or method as in any preceding claim, wherein the polymeric material is free of covalent crosslinking agents.

17. The article or method as in any preceding claim, wherein the polymeric material has a Young's modulus of 5-100 MPa at equilibrium water content (EWC).

18. The article or method as in any preceding claim, wherein the article comprises a plurality of interconnected pores.

19. The article or method as in any preceding claim, wherein the component comprises a plurality of fibers.

20. The article or method as in any preceding claim, wherein the component is a catheter cuff.

21. The article or method as in any preceding claim, wherein the body portion comprises a second material, comprising the first water-soluble polymer at a concentration less than the concentration of first water-soluble polymer in the first material.

22. The article or method as in any preceding claim, wherein the water-soluble polymer may entrain non-soluble compounds selected from the group consisting of therapeutics, biologically active compounds, woven textiles, metallic mesh, color indicators, radiopacifiers, and reactive indicators.

23. The article or method as in any preceding claim, wherein the water-soluble polymer is incorporated with conductive indicators to aid in detection of an electrocardiogram p-wave.

24. The article or method as in claim 23, wherein the conductive indicator comprises a ferrous powder that does not dissolve in physiological fluid entrained in the PVA.

25. The article or method as in any preceding claim, wherein the water-soluble polymer is entrained with a woven textile selected from the group consisting of Dacron® (Poly ethylene terephthalate aka polyester), electrospun PVA, cotton, wool, Polypropylene (atactic, syndiotactic, isotactic), and polyethylene (LLDPE, LDPE, HDPE).

26. The article or method as in any preceding claims, wherein the intended use of said article or method is selected from the group consisting of binding bandages, tissue growth, stents, shunts, braided tubing, and wall stents, to another PVA substrate.

27. The article or method as in any preceding claim, wherein the first water-soluble polymer and second water-soluble polymer are bonded together to create a new geometry or to reinforce the one or more components physically integrated with the body portion.

28. The article or method as in claim 27, wherein bonding comprises hydrating and heating the first and second water-soluble polymers to be bonded in an overlapped joint.

29. The article or method as in any preceding claim, wherein heating causes the first water-soluble polymer to flow into the second water-soluble polymer.

30. The article or method as in any preceding claims, wherein heating causes second water-soluble polymer to flow into the first water-soluble polymer.

31. The article or method as in any preceding claim, wherein bonding further involves cooling the first and second water-soluble polymers.

32. The article or method as in any preceding claims, wherein cooling the polymers forms the integrated article.

33. The article or method as in any preceding claim, wherein the water-soluble polymer is hydrated is a solution until an equilibrium % hydration is obtained.

34. The article or method as in any preceding claim, wherein the equilibrium % hydration is between about 0.1% and 100%.

35. The article or method as in any preceding claim, wherein solution comprises phosphate buffered saline at a concentration between about 0.1 M and 10 M.

36. The article or method as in any preceding claim, wherein bonding is accomplished using a split die bonder, RF welder, or hot air station of a specific geometry.

37. The article or method as in any preceding claim, wherein the bonding temperature is between 60 C to 95 C.

38. The article or method as in any preceding claim, wherein bonding time is between 15 seconds and 5 minutes.

39. The article or method as in any preceding claim, wherein bonding is accomplished using FEP heat shrink formed to the appropriate size and shape for hot air processing.

40. The article or method as in any preceding claim, wherein bonding further comprises compressing the bond to increase chain entanglement of the materials to be bonded.

41. A method for reshaping a hydrophilic porous material, comprising:
bending the hydrophilic porous material in a desired shape, the hydrophilic porous material comprising a lumen;
heating the hydrophilic porous material in the bent configuration to greater than or equal to 90° C.,
wherein:
a) the step of bending the hydrophilic porous material comprises pressing the hydrophilic porous material into a mold having the desired shape; and/or
b) the step of bending the hydrophilic porous material comprises inserting a material into the lumen of the hydrophilic porous material thereby providing the desired shape; and/or
c) the step of bending the hydrophilic porous material comprises physically deforming the hydrophilic porous material
and wherein, for any of (a)-(c), one or more of the following holds:
i) the hydrophilic porous material has a Young's elastic modulus of greater than or equal to 500 MPa in a dehydrated state and a Young's elastic modulus of less than or equal to 300 MPa and greater than or equal to 5 MPa at an equilibrium water content state;
ii) the hydrophilic porous material is configured to swell in an amount greater than or equal to 5 w/w % and less than or equal to 50 w/w % from a dehydrated state to an equilibrium water content state in less than or equal to 60 minutes at 25° C.;
iii) the hydrophilic porous material is free of covalent crosslinks between the water-soluble polymer that forms the hydrophilic porous material; and
iv) the hydrophilic porous material comprises pores that each have a diameter of 1 μm or less.

42. An article, comprising:
an elongated tube, wherein the elongated tube comprises a first material comprising a water-soluble polymer;
the elongated tube comprising a first portion and a second portion,
wherein the first portion comprises a radius of curvature different than a radius of curvature of the second portion, in a relaxed state of the elongated tube,
wherein one or more of the following holds:
v) the first material has a Young's elastic modulus of greater than or equal to 500 MPa in a dehydrated state and a Young's elastic modulus of less than or equal to 300 MPa and greater than or equal to 5 MPa at an equilibrium water content state;
vi) the first material is configured to swell in an amount greater than or equal to 5 w/w % and less than or equal to 50 w/w % from a dehydrated state to an equilibrium water content state in less than or equal to 60 minutes at 25° C.;
vii) the first material is free of covalent crosslinks between the water-soluble polymer that forms the first material; and
viii) the first material comprises pores that each have a diameter of 1 μm or less.

43. A dual lumen article, comprising:
a first body portion comprising a first material comprising a water-soluble polymer, the first body portion comprising a first lumen having a first inner diameter; and
a second body portion comprising a second material, different than the first material, the second body portion comprising a second lumen, wherein a first portion of the second body has an outer diameter less than the first inner diameter of the first lumen, and a second portion of the second body has a second inner diameter about equal to the first inner diameter,
wherein the second body portion is at least partially disposed within the first body portion,
wherein one or more of the following holds:
ix) the first material has a Young's elastic modulus of greater than or equal to 500 MPa in a dehydrated state and a Young's elastic modulus of less than or equal to 300 MPa and greater than or equal to 5 MPa at an equilibrium water content state;
x) the first material is configured to swell in an amount greater than or equal to 5 w/w % and less than or equal to 50 w/w % from a dehydrated state to an equilibrium water content state in less than or equal to 60 minutes at 25° C.;
xi) the first material is free of covalent crosslinks between the water-soluble polymer that forms the first material; and
xii) the first material comprises pores that each have a diameter of 1 μm or less.

44. A method of forming a dual lumen article, comprising:
swelling, in a solvent, a first body portion comprising a first material comprising a water-soluble polymer, the first body portion comprising a first lumen having a first inner diameter;
heating and/or mechanically deforming a second body portion, the second body portion comprising a second material different than the first material, such that the second body portion has an outer diameter less than the first inner diameter of the first lumen;
inserting the second body portion into the lumen of the first body portion thereby forming the dual lumen article; and
drying the dual lumen article such that the first body portion shrinks.

45. The article or method as in any preceding claim, further comprising one or more surface features (e.g., barbs, bulges) at an interface between the first body portion and the second body portion, the one or more surface features configured to mechanically reinforce the interface between the first body portion and the second body portion.

46. The article or method as in any preceding claim, further comprising one or more surface features (e.g., barbs, bulges) at an interface between the first body portion and the second body portion, the one or more surface features configured to mechanically reinforce the interface between the first body portion and the second body portion.

47. The article or method as in any preceding claim, wherein the article is shaped using thermal forming.

48. The article or method as in any preceding claim, wherein thermal forming occurs at a temperature greater than 90 C.

49. The article or method as in any preceding claim, wherein thermal forming comprising placing a wire into the lumen of the article, placing the article into a mold, and thermally heating the mold into a designed shape.

50. The article or method as in any preceding claim, wherein the article shape is curved, straightened, compressed, or stretched by restricting the dimensions of the initial component to the desired shape.

51. The article or method as in any preceding claim, wherein the article edges can be rounded to allow ease of fluid flow, reduction of abrasion, and reduction of surface area, which can aid placement, catheter longevity, and patient comfort.

52. The article or method of any preceding claim, wherein the solvent comprises water.

53. The article or method of any preceding claim, wherein the solvent comprises water and the water-soluble polymer.

54. The article or method of any preceding claim, wherein the solvent comprises the water-soluble polymer at a concentration less than the concentration of the water-soluble polymer used to form the first material.

55. The article or method of any preceding claim, wherein the solvent comprises water and polyvinyl alcohol, the polyvinyl alcohol present in the solvent in an amount of greater than or equal to 0.1 wt % and less than or equal to 25 wt %.

56. The article or method of any preceding claim, wherein the solvent has hydrolysis levels of greater than or equal to 80%, such that the solvent comprises less than or equal to 20% acetate groups.

57. The article as in any preceding claim, wherein the article comprises a lumen.

58. The article as in any preceding claim, wherein the article comprises a hemodialysis catheter, a peripherally inserted central catheter (PICC), a central venous catheter, a midline catheter, a peripheral catheter, a urinary catheter, a neurological catheter, a peritoneal catheter, and/or an infusion port.

59. The article as in any preceding claim, wherein the article is configured for use with a medical article such as a catheter, a balloon, a shunt, a wound drain, an infusion port, a drug delivery article, a tube, a contraceptive article, a feminine hygiene article, an endoscope, a grafts, a pacemaker, an implantable cardioverter-defibrillator, a cardiac resynchronization article, a cardiovascular article lead, a ventricular assist article, an endotracheal tube, a tracheostomy tube, an implantable sensor, a ventilator pump, and an ophthalmic article

60. The article or method as in any preceding claim, wherein the polymeric material is free of covalent crosslinking agents.

61. The article or method as in any preceding claim, wherein the polymeric material has a Young's modulus of 5-100 MPa at equilibrium water content (EWC).

62. The article or method as in any preceding claim, wherein the article comprises a plurality of interconnected pores.

63. The article or method as in any preceding claim, wherein the component comprises a plurality of fibers.

64. The article or method as in any preceding claim, wherein the component is a catheter cuff.

65. The article or method as in any preceding claim, wherein the body portion comprises a second material, comprising the first water-soluble polymer at a concentration less than the concentration of first water-soluble polymer in the first material.

66. The article or method as in any preceding claim, wherein the water-soluble polymer may entrain non-soluble compounds selected from the group consisting of therapeutics, biologically active compounds, woven textiles, metallic mesh, color indicators, radiopacifiers, and reactive indicators.

67. The article or method as in any preceding claim, wherein the water-soluble polymer is incorporated with conductive indicators to aid in detection of an electrocardiogram p-wave.

68. The article or method as in claim 23, wherein the conductive indicator comprises a ferrous powder that does not dissolve in physiological fluid entrained in the PVA.

69. The article or method as in any preceding claim, wherein the water-soluble polymer is entrained with a woven textile selected from the group consisting of Dacron® (Poly ethylene terephthalate aka polyester), electrospun PVA, cotton, wool, Polypropylene (atactic, syndiotactic, isotactic), and polyethylene (LLDPE, LDPE, HDPE).

70. The article or method as in any preceding claims, wherein the intended use of said article or method is selected from the group consisting of binding bandages, tissue growth, stents, shunts, braided tubing, and wall stents, to another PVA substrate.

71. The article or method as in any preceding claim, wherein the first water-soluble polymer and second water-soluble polymer are bonded together to create a new geometry or to reinforce the one or more components physically integrated with the body portion.

72. The article or method as in claim 27, wherein bonding comprises hydrating and heating the first and second water-soluble polymers to be bonded in an overlapped joint.

73. The article or method as in any preceding claim, wherein heating causes the first water-soluble polymer to flow into the second water-soluble polymer.

74. The article or method as in any preceding claims, wherein heating causes second water-soluble polymer to flow into the first water-soluble polymer.

75. The article or method as in any preceding claim, wherein bonding further involves cooling the first and second water-soluble polymers.

76. The article or method as in any preceding claims, wherein cooling the polymers forms the integrated article.

77. The article or method as in any preceding claim, wherein the water-soluble polymer is hydrated is a solution until an equilibrium % hydration is obtained.

78. The article or method as in any preceding claim, wherein the equilibrium % hydration is between about 0.1% and 100%.

79. The article or method as in any preceding claim, wherein solution comprises phosphate buffered saline at a concentration between about 0.1 M and 10 M.

80. The article or method as in any preceding claim, wherein bonding is accomplished using a split die bonder, RF welder, or hot air station of a specific geometry.

81. The article or method as in any preceding claim, wherein the bonding temperature is between 60 C to 95 C.

82. The article or method as in any preceding claim, wherein bonding time is between 15 seconds and 5 minutes.

83. The article or method as in any preceding claim, wherein bonding is accomplished using FEP heat shrink formed to the appropriate size and shape for hot air processing.

84. The article or method as in any preceding claim, wherein bonding further comprises compressing the bond to increase chain entanglement of the materials to be bonded.

85. An intravenous catheter, comprising:
a polymeric material comprising a first water-soluble polymer having a plurality of pores;
a lumen; and
a distal end comprising a tip geometry suitable for intravenous insertion into a subject;
wherein the polymeric material has a water content of less than 5 w/w % and greater than or equal to 0.1 w/w % in a dehydrated state, and wherein the polymeric material is configured to swell in an amount greater than or equal to 5 w/w % and less than or equal to 50 w/w % from a dehydrated state to an equilibrium water content state in less than or equal to 60 minutes at 25° C.

86. A system comprising the intravenous catheter as in any preceding claim, and a component configured for administering a therapeutic agent and/or fluid to a subject, and/or drawing a bodily fluid from the subject.

87. A method, comprising:
inserting, intravenously, an intravenous catheter, the intravenous catheter comprising:
a polymeric material comprising a first water-soluble polymer having a plurality of pores;
a lumen; and
a distal end comprising a tip geometry suitable for intravenous insertion into a subject;
wherein, during the step of insertion, the catheter has a water content of less than or equal to 5 w/w %; and
swelling the intravenous catheter to a water content greater than or equal to 5 w/w % and less than or equal to 50 w/w %.

88. A method, comprising:
inserting, intravenously, an intravenous catheter, the intravenous catheter comprising:
a polymeric material comprising a first water-soluble polymer having a plurality of pores, wherein the polymeric material has a water content greater than or equal to 5 w/w % and less than or equal to 50 w/w %;
a lumen; and
a distal end comprising a tip geometry suitable for intravenous insertion into a subject;
wherein one or more of the following holds:
i) the polymeric material has a Young's elastic modulus of greater than or equal to 500 MPa in a dehydrated state and a Young's elastic modulus of less than or equal to 300 MPa and greater than or equal to 5 MPa at an equilibrium water content state;
ii) the polymeric material is free of covalent crosslinks between the water-soluble polymer that forms the polymeric material; and
iii) the polymeric material comprises pores that each have a diameter of 1 μm or less.

89. The catheter or method as in any preceding claim, wherein the polymeric material is free of covalent crosslinking agents.

90. The catheter or method as in any preceding claim, wherein the article comprises a plurality of interconnected pores,

91. The catheter or method as in any preceding claim, wherein the polymeric material has a Young's elastic modulus of greater than or equal to 500 MPa in a dehydrated state and a Young's elastic modulus of less than or equal to 300 MPa and greater than or equal to 5 MPa at an equilibrium water content state.

92. The catheter or method as in any preceding claim, wherein the intravenous catheter is configured for insertion into a subject in the dehydrated state.

93. The catheter or method as in any preceding claim, wherein the intravenous catheter is configured for insertion into a subject in an at least partially hydrated state.

94. The catheter or method as in any preceding claim, further comprising administering a fluid to the subject via the intravenous catheter.

95. The catheter or method as in any preceding claim, further comprising drawing blood from the subject via the intravenous catheter.

96. The catheter or method as in any preceding claim, wherein the intravenous catheter exhibits hemolysis in an amount of less than or equal to 10%.

97. The catheter or method as in any preceding claim, wherein the intravenous catheter comprises a softening ratio for a 3-point ratio of at least 99% reduction in force in insertion versus final hydrated state.

98. The catheter or method as in any preceding claim, wherein the distal end of the catheter is tipped.

99. The catheter or method as in any preceding claim, wherein the catheter proximal end of the catheter is hubbed.
